(19) United States
(12) Patent Application Publication
(10) Pub. No.: US 2021/0111350 A1
(43) Pub. Date: Apr. 15, 2021

(54) PHOSPHORESCENT HOST COMPOSITION, ORGANIC OPTOELECTRONIC DIODE, AND DISPLAY DEVICE

(71) Applicant: SAMSUNG SDI CO., LTD., Yongin-si, Gyeonggi-do (KR)

(72) Inventors: Jinhyun LUI, Suwon-si, Gyeonggi-do (KR); Jaehan PARK, Suwon-si, Gyeonggi-do (KR); Ho Kuk JUNG, Suwon-si, Gyeonggi-do (KR); Pyeongseok CHO, Suwon-si, Gyeonggi-do (KR); Dong Min KANG, Suwon-si, Gyeonggi-do (KR); Sangshin LEE, Suwon-si, Gyeonggi-do (KR); Seungjae LEE, Suwon-si, Gyeonggi-do (KR); Kipo JANG, Suwon-si, Gyeonggi-do (KR); Sung-Hyun JUNG, Suwon-si, Gyeonggi-do (KR); Juyeon JUNG, Suwon-si, Gyeonggi-do (KR); Handong CHU, Suwon-si, Gyeonggi-do (KR)

(21) Appl. No.: 16/498,616

(22) PCT Filed: May 25, 2018
(86) PCT No.: PCT/KR2018/005989

§ 371 Date: Sep. 27, 2019

(30) Foreign Application Priority Data
May 26, 2017 (KR) 10-2017-0065468
May 21, 2018 (KR) 10-2018-0057825

(51) Int. Cl. H01L 51/00; C09K 11/02; C09K 11/06; C07D 405/14; C07D 405/04; C07D 487/04

(52) U.S. Cl. CPC H01L 51/0067; H01L 51/5016; C09K 11/06; C07D 405/14; H01L 51/0073; C07D 405/04; H01L 51/0052; C07D 487/04; H01L 51/0072; H01L 51/0085; H01L 51/0087; C09K 2211/185; C09K 2211/1029; C09K 2211/1007; C09K 11/02

(57) ABSTRACT

Provided are a composition for a phosphorescent host including a first host represented by Chemical Formula 1, a second host represented by a combination of Chemical Formula 2 and Chemical Formula 3; and an organic optoelectronic device including an anode and a cathode facing each other and an organic layer disposed between the anode and the cathode, wherein the organic layer includes an auxiliary layer including at least one of a hole injection layer, a hole transport layer, an electron injection layer, and an electron transport layer and a light emitting layer, and the light emitting layer includes a phosphorescent dopant having a maximum photoluminescence wavelength of 550 nm to 750 nm along with the composition, and a display device including the same. Details of Chemical Formulae 1 to 3 are the same as defined in the specification.

### TECHNICAL FIELD

A composition for a phosphorescent host, an organic optoelectronic device, and a display device are disclosed.

### BACKGROUND ART

An organic optoelectronic device (organic optoelectronic diode) is a device that converts electrical energy into photoenergy, and vice versa.

An organic optoelectronic device may be classified as follows in accordance with its driving principles. One is a photoelectric device where excitons are generated by photoenergy, separated into electrons and holes, and are transferred to different electrodes to generate electrical energy, and the other is a light emitting device where a voltage or a current is supplied to an electrode to generate photoenergy from electrical energy.

Examples of the organic optoelectronic diode may be an organic photoelectric device, an organic light emitting diode, an organic solar cell, and an organic photo conductor drum.

Of these, an organic light emitting diode (OLED) has recently drawn attention due to an increase in demand for flat panel displays. The organic light emitting diode is a device converting electrical energy into light by applying current to an organic light emitting material, and has a structure in which an organic layer is disposed between an anode and a cathode. Herein, the organic layer may include a light emitting layer and optionally an auxiliary layer, and the auxiliary layer may be, for example at least one layer selected from a hole injection layer, a hole transport layer, an electron blocking layer, an electron transport layer, an electron injection layer, and a hole blocking layer.

Performance of an organic light emitting diode may be affected by characteristics of the organic layer, and among them, may be mainly affected by characteristics of an organic material of the organic layer.

Particularly, development for an organic material being capable of increasing hole and electron mobility and simultaneously increasing electrochemical stability is needed so that the organic light emitting diode may be applied to a large-size flat panel display.

### DISCLOSURE

### Technical Problem

An embodiment provides a composition for a phosphorescent host capable of embodying an organic optoelectronic device having high efficiency and long life-span.

Another embodiment provides an organic optoelectronic device including the composition.

Yet another embodiment provides a display device including the organic optoelectronic device.

### Technical Solution

According to an embodiment, an organic optoelectronic device includes an anode and a cathode facing each other and an organic layer disposed between the anode and the cathode, wherein the organic layer includes an auxiliary layer including at least one of a hole injection layer, a hole transport layer, an electron injection layer, and an electron transport layer, and a light emitting layer, and the light emitting layer includes a first host represented by Chemical Formula 1, a second host represented by a combination of Chemical Formula 2 and Chemical Formula 3, and a phosphorescent dopant having a maximum photoluminescence wavelength of 550 nm to 750 nm.

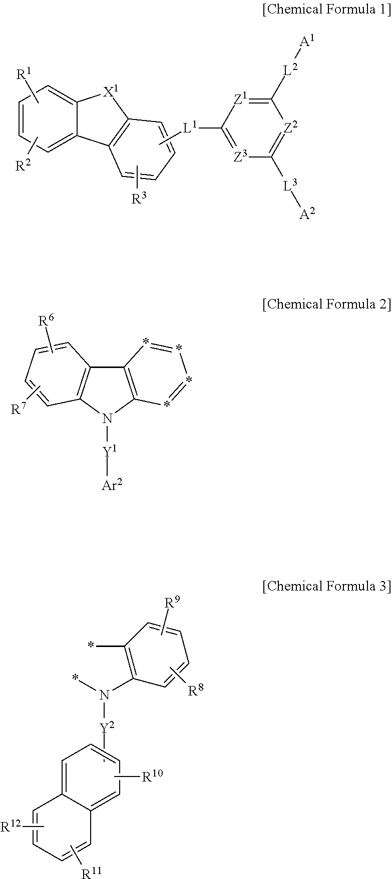

In Chemical Formula 1,

X¹ is O or S,

Z¹ to Z³ are independently N or CR^{a},

at least two of Z¹ to Z³ are N,

L¹ to L³ are independently a single bond, or a substituted or unsubstituted C6 to C20 arylene group,

A¹ and A² are independently a substituted or unsubstituted C6 to C30 aryl group, or a substituted or unsubstituted C2 to C30 heterocyclic group,

at least one of A¹ and A² is a substituted or unsubstituted C6 to C30 aryl group,

R^{a} and R¹ to R³ are independently hydrogen, deuterium, a cyano group, a substituted or unsubstituted C1 to C10 alkyl group, or a substituted or unsubstituted C6 to C20 aryl group;
wherein, in Chemical Formulae 2 and 3,

Ar² is a substituted or unsubstituted C6 to C20 aryl group,

adjacent two *'s of Chemical Formula 2 are linked with Chemical Formula 3,

* of Chemical Formula 2 that are not linked with Chemical Formula 3 are independently C-L^{a}-R^{b},

L^{a}, Y¹, and Y² are independently a single bond, or a substituted or unsubstituted C6 to C20 arylene group, and

R^{b} and R⁶ to R¹² are independently hydrogen, deuterium, a cyano group, a substituted or unsubstituted C1 to C10 alkyl group, or a substituted or unsubstituted C6 to C20 aryl group.

According to another embodiment, a composition for a red phosphorescent host including the first host represented by Chemical Formula 1 and the second host represented by a combination of Chemical Formula 2 and Chemical Formula 3 is provided.

According to another embodiment, a display device including the organic optoelectronic device is provided.

### Advantageous Effects

An organic optoelectronic device having high efficiency and a long life-span may be realized.

### DESCRIPTION OF THE DRAWINGS

FIGS. 1 and 2 are cross-sectional views showing organic light emitting diodes according to embodiments.

### DESCRIPTION OF SYMBOLS

100, 200: organic light emitting diode
105: organic layer
110: cathode
120: anode
130: light emitting layer
140: hole auxiliary layer

### BEST MODE

Hereinafter, embodiments of the present invention are described in detail. However, these embodiments are exemplary, the present invention is not limited thereto and the present invention is defined by the scope of claims.

In the present specification when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to 010 trifluoroalkyl group, a cyano group, or a combination thereof.

In one example of the present invention, “substituted” refers to replacement of at least one hydrogen of a substituent or a compound by deuterium, a cyano group, a C1 to 010 alkyl group, a C6 to C20 aryl group, or a C2 to C20 heterocyclic group. In addition, in specific examples of the present invention, “substituted” refers to replacement of at least one hydrogen of a substituent or a compound by deuterium, a cyano group, a C1 to C4 alkyl group, a C6 to C12 aryl group, or a C2 to C12 heterocyclic group. More specifically, “substituted” refers to replacement of at least one hydrogen of a substituent or a compound by deuterium, a cyano group, a C1 to C5 alkyl group, a phenyl group, a biphenyl group, a terphenyl group, a naphthyl group, a fluorenyl group, a benzofuranyl group, a benzothiophenyl group, a dibenzofuranyl group, a dibenzothiophenyl group, or a carbazolyl group. In addition, in most specific examples of the present invention, “substituted” refers to replacement of at least one hydrogen of a substituent or a compound by deuterium, a cyano group, a methyl group, an ethyl group, a propanyl group, a butyl group, a phenyl group, a para-biphenyl group, a meta-biphenyl group, a dibenzofuranyl group, or a dibenzothiophenyl group.

In the present specification, when a definition is not otherwise provided, “hetero” refers to one including one to three heteroatoms selected from N, O, S, P, and Si, and remaining carbons in one functional group.

In the present specification, when a definition is not otherwise provided, “alkyl group” refers to an aliphatic hydrocarbon group. The alkyl group may be “a saturated alkyl group” without any double bond or triple bond.

The alkyl group may be a C1 to C30 alkyl group. More specifically, the alkyl group may be a C1 to C20 alkyl group or a C1 to C10 alkyl group. For example, a C1 to C4 alkyl group may have one to four carbon atoms in the alkyl chain, and may be selected from methyl, ethyl, propyl, iso-propyl, n-butyl, iso-butyl, sec-butyl, and t-butyl.

Specific examples of the alkyl group may be a methyl group, an ethyl group, a propyl group, an isopropyl group, a butyl group, an isobutyl group, a t-butyl group, a pentyl group, a hexyl group, a cyclopropyl group, a cyclobutyl group, a cyclopentyl group, a cyclohexyl group, and the like.

In the present specification, “an aryl group” refers to a group including at least one hydrocarbon aromatic moiety, and

all elements of the hydrocarbon aromatic moiety have p-orbitals which form conjugation, for example a phenyl group, a naphthyl group, and the like,

two or more hydrocarbon aromatic moieties may be linked by a sigma bond and may be, for example a biphenyl group, a terphenyl group, a quarterphenyl group, and the like, and

two or more hydrocarbon aromatic moieties are fused directly or indirectly to provide a non-aromatic fused ring. For example, it may be a fluorenyl group.

The aryl group may include a monocyclic, polycyclic, or fused ring polycyclic (i.e., rings sharing adjacent pairs of carbon atoms) functional group.

In the present specification, “a heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

Specific examples of the heterocyclic group may include a pyridinyl group, a pyrimidinyl group, a pyrazinyl group, a pyridazinyl group, a triazinyl group, a quinolinyl group, an isoquinolinyl group, and the like.

More specifically, the substituted or unsubstituted C6 to C30 aryl group and/or the substituted or unsubstituted C2 to C30 heterocyclic group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, or combination thereof, but are not limited thereto.

In the present specification, hole characteristics refer to an ability to donate an electron to form a hole when an electric field is applied and that a hole formed in the anode may be easily injected into the light emitting layer, a hole formed in the light emitting layer may be easily transported into the anode, and a hole may be easily transported in the light emitting layer due to conductive characteristics according to a highest occupied molecular orbital (HOMO) level.

In addition, electron characteristics refer to an ability to accept an electron when an electric field is applied and that an electron formed in the cathode may be easily injected into the light emitting layer, an electron formed in the light emitting layer may be easily transported into the cathode, and an electron may be easily transported in the light emitting layer due to conductive characteristics according to a lowest unoccupied molecular orbital (LUMO) level.

Hereinafter, an organic optoelectronic device according to an embodiment is described.

The organic optoelectronic device may be any device to convert electrical energy into photoenergy and vice versa without particular limitation, and may be for example an organic photoelectric device, an organic light emitting diode, an organic solar cell, and an organic photo conductor drum, and the like.

Herein, an organic light emitting diode as one example of an organic optoelectronic device is described referring to drawings.

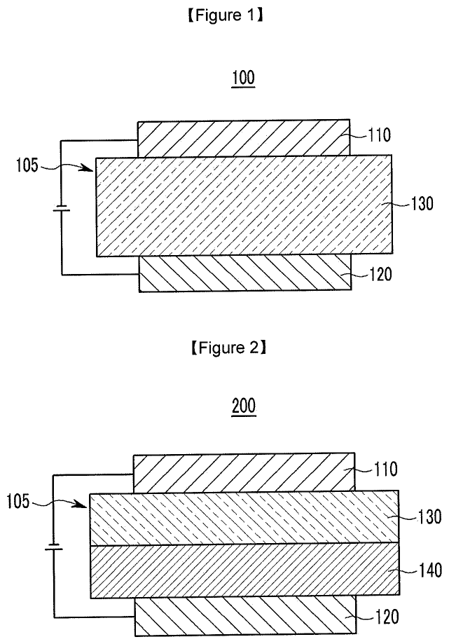

FIGS. 1 and 2 are cross-sectional view showing organic light emitting diodes according to embodiments.

Referring to FIG. 1, an organic light emitting diode 100 according to an embodiment includes an anode 120 and a cathode 110 facing each other and an organic layer 105 interposed between the anode 120 and cathode 110.

The anode 120 may be made of a conductor having a large work function to help hole injection, and may be for example metal, metal oxide and/or a conductive polymer. The anode 120 may be, for example a metal such as nickel, platinum, vanadium, chromium, copper, zinc, gold and the like or an alloy thereof; metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example metal, metal oxide and/or a conductive polymer. The cathode 110 may be for example a metal such as magnesium, calcium, sodium, potassium, titanium, indium, yttrium, lithium, gadolinium, aluminum silver, tin, lead, cesium, barium, and the like or an alloy thereof; a multi-layer structure material such as LiF/Al, LiO₂/Al, LiF/Ca, LiF/Al and BaF₂/Ca, but is not limited thereto.

The organic layer 105 includes an auxiliary layer including at least one of a hole injection layer, a hole transport layer, an electron injection layer, and an electron transport layer and a light emitting layer 130.

FIG. 2 is a cross-sectional view showing an organic light emitting diode according to another embodiment.

Referring to FIG. 2, an organic light emitting diode 200 further includes a hole auxiliary layer 140 in addition to the light emitting layer 130. The hole auxiliary layer 140 may further increase hole injection and/or hole mobility and block electrons between the anode 120 and the light emitting layer 130. The hole auxiliary layer 140 may be, for example a hole transport layer, a hole injection layer, and/or an electron blocking layer and may include at least one layer.

The organic layer 105 of FIG. 1 or 2 may further include an electron injection layer, an electron transport layer, an electron transport auxiliary layer, a hole transport layer, a hole transport auxiliary layer, a hole injection layer, or a combination thereof even if they are not shown.

The organic light emitting diodes 100 and 200 may be manufactured by forming an anode or a cathode on a substrate, forming an organic layer using a dry film formation method such as a vacuum deposition method (evaporation), sputtering, plasma plating, and ion plating or a wet coating method such as spin coating, dipping, and flow coating, and forming a cathode or an anode thereon.

An organic optoelectronic device according to an embodiment includes an anode and a cathode facing each other, and

an organic layer disposed between the anode and the cathode, wherein the organic layer includes an auxiliary layer including at least one of a hole injection layer, a hole transport layer, an electron injection layer, and an electron transport layer, and a light emitting layer, and the light emitting layer includes a first host represented by Chemical Formula 1, a second host represented by a combination of Chemical Formula 2 and Chemical Formula 3, and a phosphorescent dopant having a maximum photoluminescence wavelength of 550 nm to 750 nm.

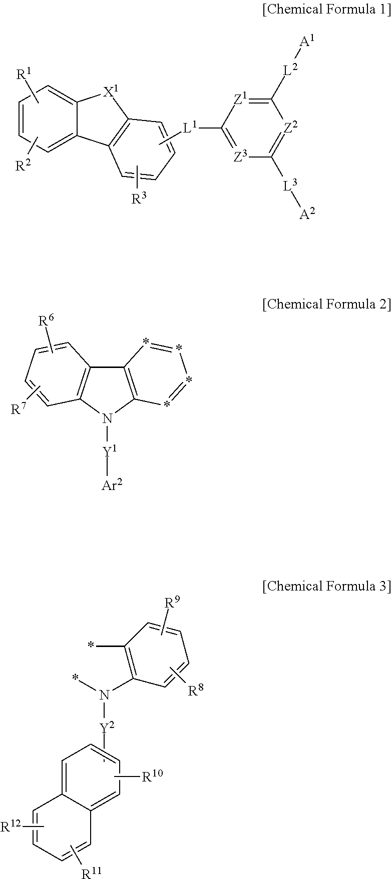

In Chemical Formula 1,

X¹ is O or S,

Z¹ to Z³ are independently N or CR^{a},

at least two of Z¹ to Z³ are N,

L¹ to L³ are independently a single bond, or a substituted or unsubstituted C6 to C20 arylene group,

A¹ and A² are independently a substituted or unsubstituted C6 to C30 aryl group, or a substituted or unsubstituted C2 to C30 heterocyclic group,

at least one of A¹ and A² is a substituted or unsubstituted C6 to C30 aryl group,

R^{a} and R¹ to R³ are independently hydrogen, deuterium, a cyano group, a substituted or unsubstituted C1 to C10 alkyl group, or a substituted or unsubstituted C6 to C20 aryl group;
wherein, in Chemical Formulae 2 and 3,

Ar² is a substituted or unsubstituted C6 to C20 aryl group,

adjacent two *'s of Chemical Formula 2 are linked with Chemical Formula 3,

* of Chemical Formula 2 that are not linked with Chemical Formula 3 are independently C-L^{a}-R^{b},

L^{a}, Y¹ and Y² are independently a single bond, or a substituted or unsubstituted C6 to C20 arylene group, and

R^{b} and R⁶ to R¹² are independently hydrogen, deuterium, a cyano group, a substituted or unsubstituted C1 to C10 alkyl group, or a substituted or unsubstituted C6 to C20 aryl group.

The organic optoelectronic device according to the present invention includes a structure where dibenzofuran (or dibenzothiophene) is linked with a triazine or pyrimidine moiety as a first host and thus may increase an injection rate of holes and electrons through expansion of LUMO and planarity expansion of an ET moiety. In addition, a planarity of molecule may be increased, and intermolecular π-πstacking may be increased by introducing fused aryl group such as naphthyl group or fused heteroaryl group as a substituent of the triazine moiety or pyrimidine moiety, and thus resultantly, a charge may transfer easily, and thus realize more advantageous driving voltage, life span and efficiency characterstics.

Particularly, a compound of the second host has an expanded HOMO electron cloud and an advantageous structure of hopping holes by introducing indolocarbazole substituted with a naphthyl group, compared with a structure having nonfused aryl alone, and thus resultantly, may secure a high hole mobility and a high glass transition temperature and thermal stability relative to molecular weight and thus, realize long life-span characteristics in a red region having a maximum photoluminescence wavelength of 550 nm to 750 nm.

In an example embodiment of the present invention, Z¹ to Z³ may be all N.

In an example embodiment of the present invention, R¹ to R³ may independently be hydrogen or a phenyl group.

In an example embodiment of the present invention, A¹ and A² of Chemical Formula 1 may independently be a substituted or unsubstituted C6 to C30 aryl group, or a substituted or unsubstituted C2 to C30 heterocyclic group, and one of A¹ and A² is a substituted or unsubstituted C6 to C30 aryl group.

In a specific example embodiment of the present invention, A¹ may be a substituted or unsubstituted C6 to C30 aryl group and A² may be a substituted or unsubstituted C6 to C30 aryl group, or a substituted or unsubstituted C2 to C30 heterocyclic group.

In a more specific example embodiment of the present invention, A¹ may be a substituted or unsubstituted C6 to C20 aryl group, and Al may be for example a substituted or unsubstituted phenyl group, a substituted or unsubstituted biphenyl group, a substituted or unsubstituted terphenyl group, a substituted or unsubstituted quaterphenyl group, or a substituted or unsubstituted naphthyl group, and Chemical Formula 1 may be represented by Chemical Formula 1-I.

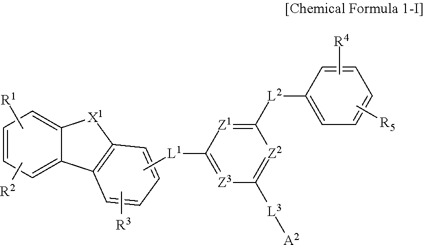

In Chemical Formula 1-I, definitions of X¹, Z¹ to Z³, L¹ to L³, A² and R¹ to R³ are the same as described above and definitions of R⁴ and R⁵ are the same as definitions of R¹ to R³.

A¹ of Chemical Formula 1 may be for example selected from substituents of Group I.

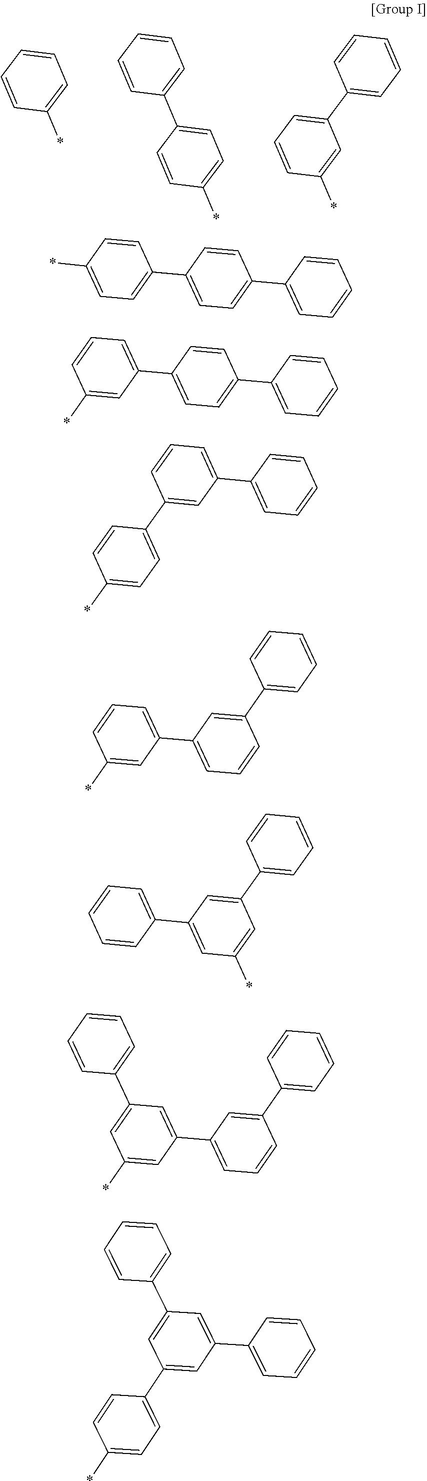

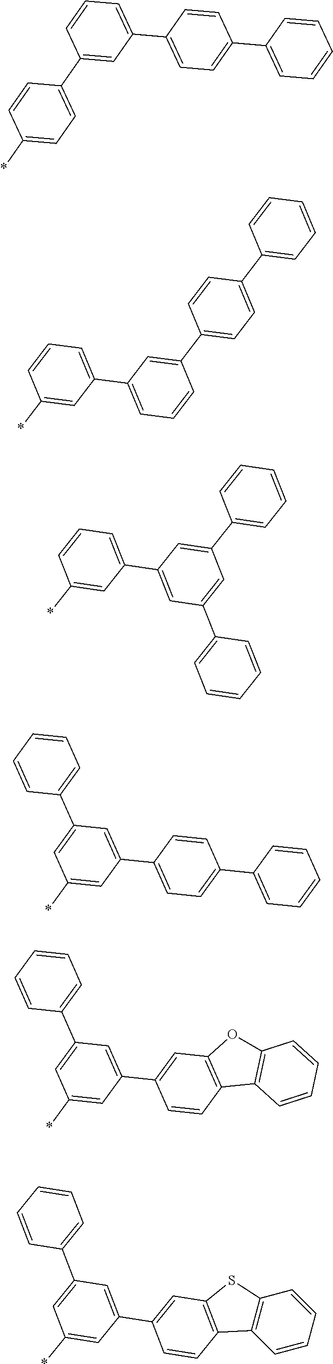

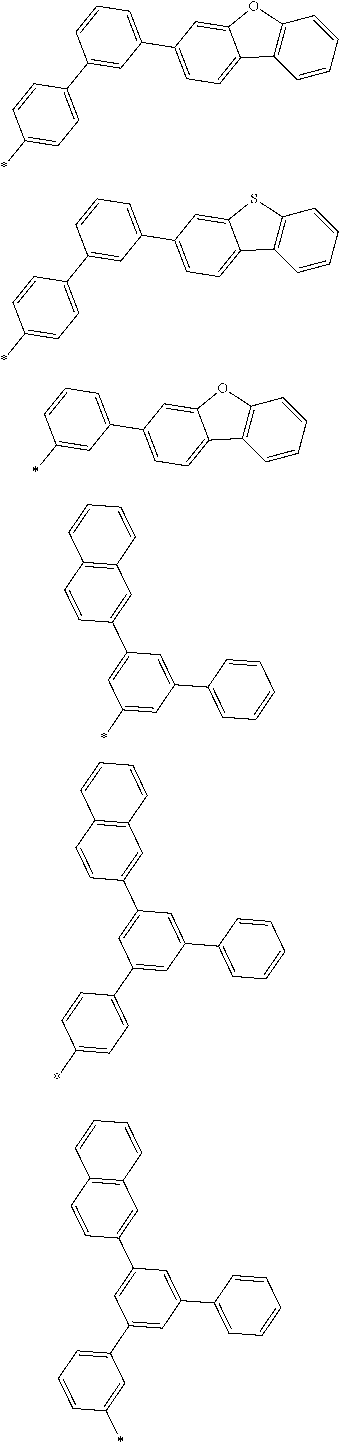

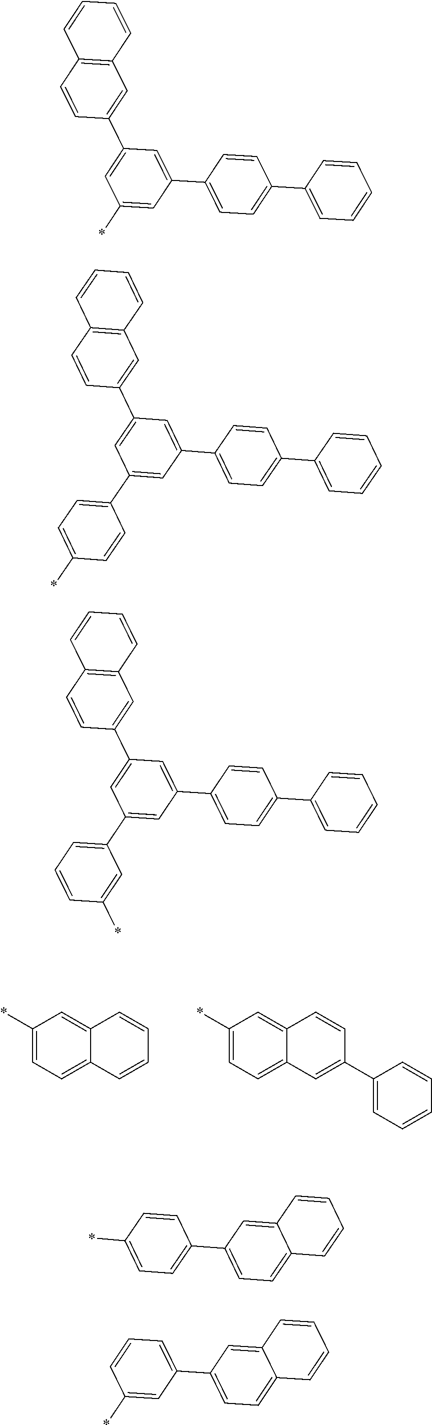

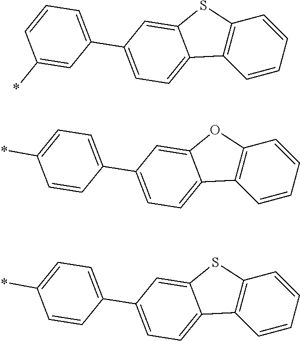 [SMILES: *c1cccc(-c2ccc3c(c2)sc2ccccc23)c1 ; *c1ccc(-c2ccc3c(c2)oc2ccccc23)cc1 ; *c1ccc(-c2ccc3c(c2)sc2ccccc23)cc1]

In Group I, * is a linking point with L².

On the other hand, A² may be a substituted or unsubstituted phenyl group, a substituted or unsubstituted biphenyl group, a substituted or unsubstituted naphthyl group, a substituted or unsubstituted terphenyl group, a substituted or unsubstituted quaterphenyl group, a substituted or unsubstituted dibenzofuranyl group, a substituted or unsubstituted dibenzothiophenyl group, a substituted or unsubstituted pyrimidinyl group, or a substituted or unsubstituted triazinyl group, and

particularly, the first host may be represented by one of Chemical Formula 1-I-1 to Chemical Formula 1-I-3 according to specific kinds of A².

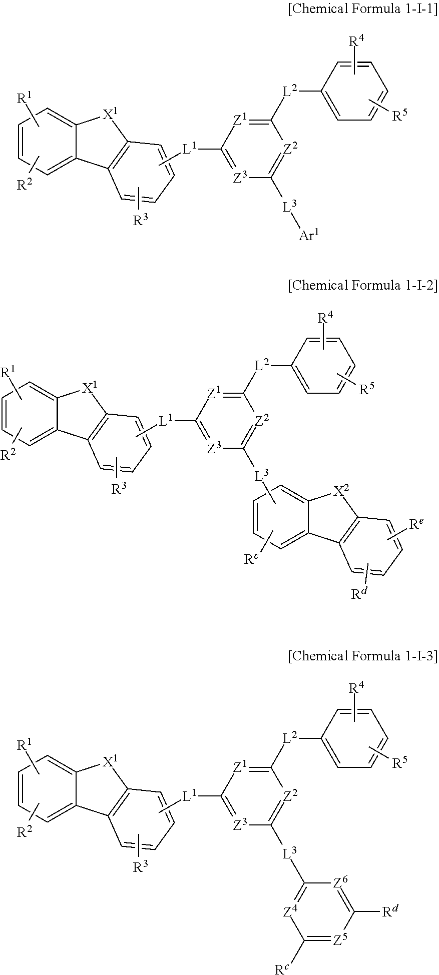

In Chemical Formula 1-I-1 to Chemical Formula 1-I-3, definitions of X¹, Z¹ to Z³, L¹ to L³, and R¹ to R⁵ are the same as described above, X² is the same as X¹, Z⁴ to Z⁶ are the same as definitions of Z¹ to Z³, and definitions of R^{c}, R^{d}, and R^{e} are the same as definitions of R¹ to R⁵.

In addition, Ar¹ of Chemical Formula 1-I-1 may be a substituted or unsubstituted C6 to C20 aryl group, and specifically a substituted or unsubstituted phenyl group, a substituted or unsubstituted biphenyl group, a substituted or unsubstituted naphthyl group, a substituted or unsubstituted terphenyl group, or a substituted or unsubstituted quaterphenyl group, wherein additional substituents may be deuterium, a cyano group, a phenyl group, or a naphthyl group.

In a specific example embodiment of the present invention, R^{c} and R^{d} of Chemical Formula 1-I-3 may independently be a substituted or unsubstituted C6 to C20 aryl group, and more specifically a phenyl group, a biphenyl group, a naphthyl group, or a terphenyl group.

A² of Chemical Formula 1 may be for example selected from substituents of Group II.

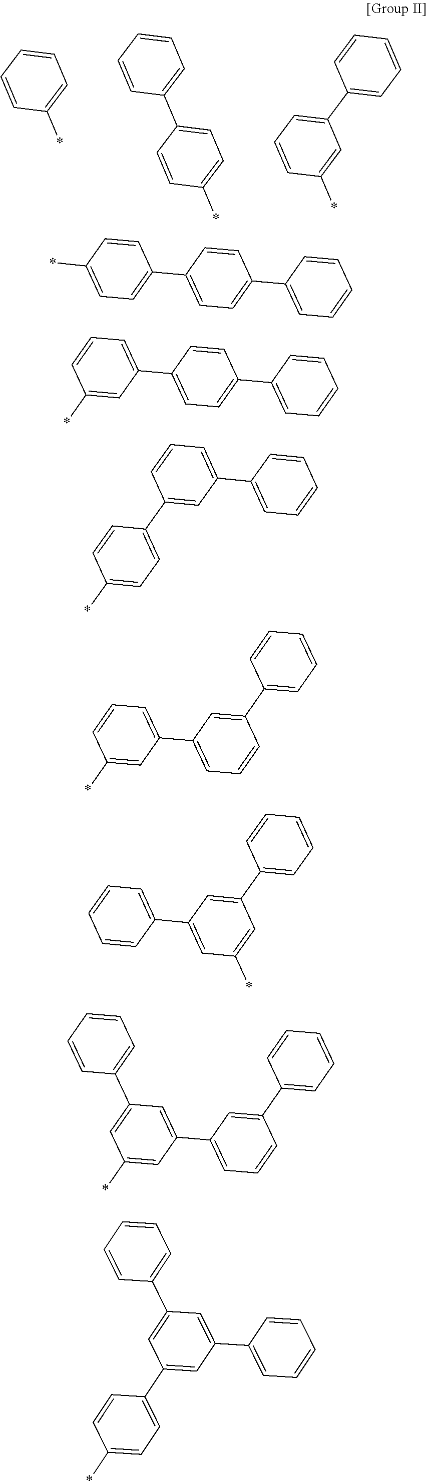

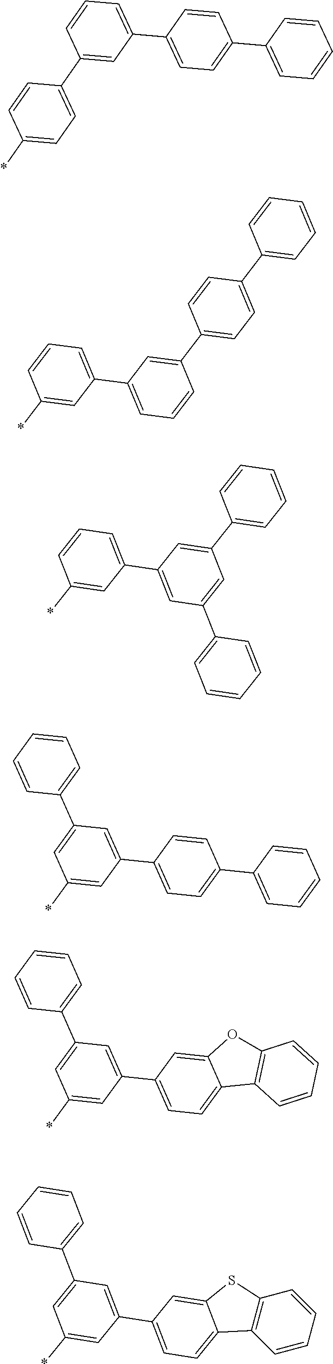

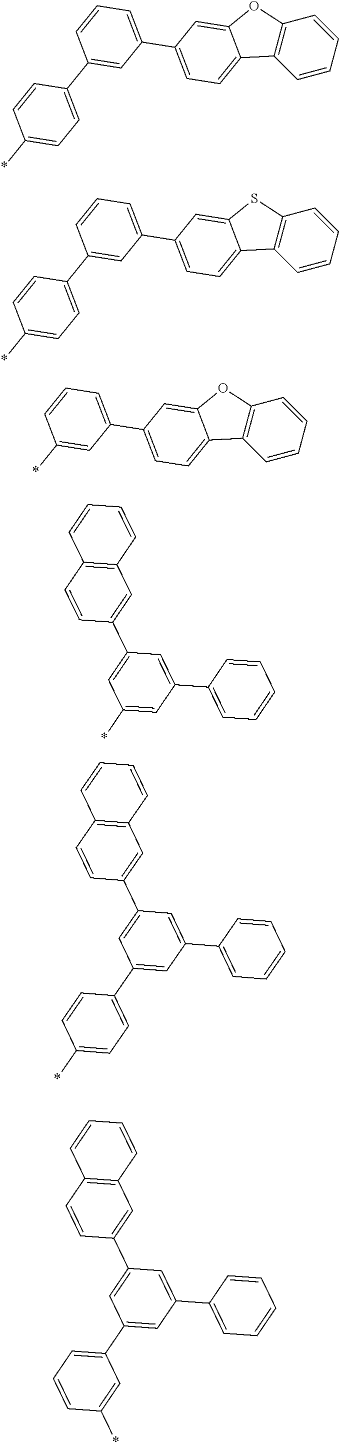

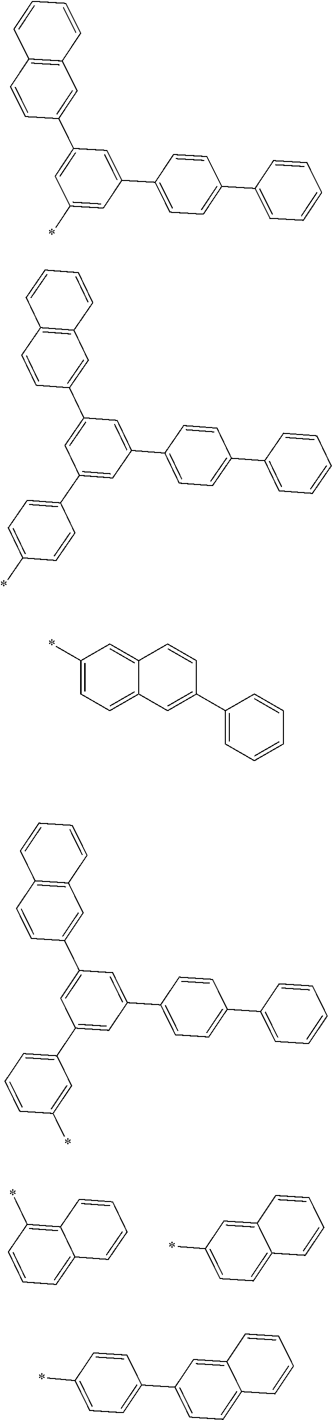

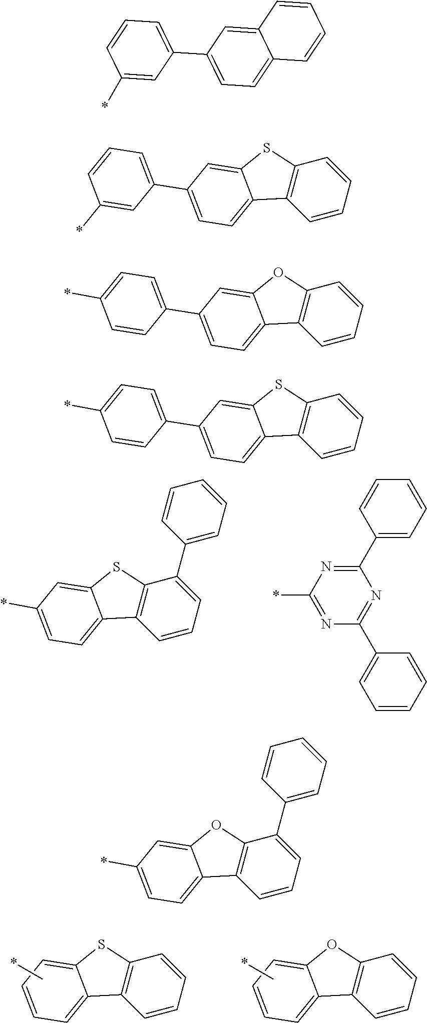

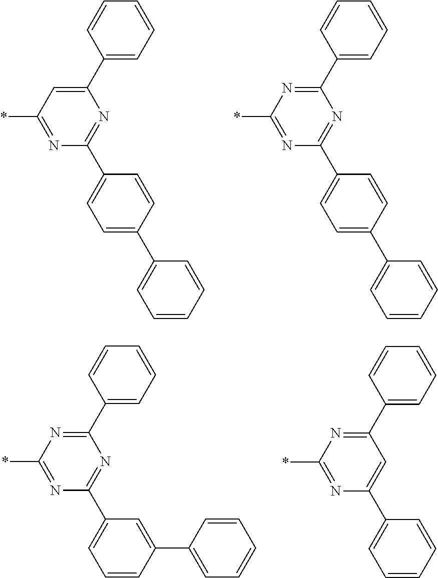

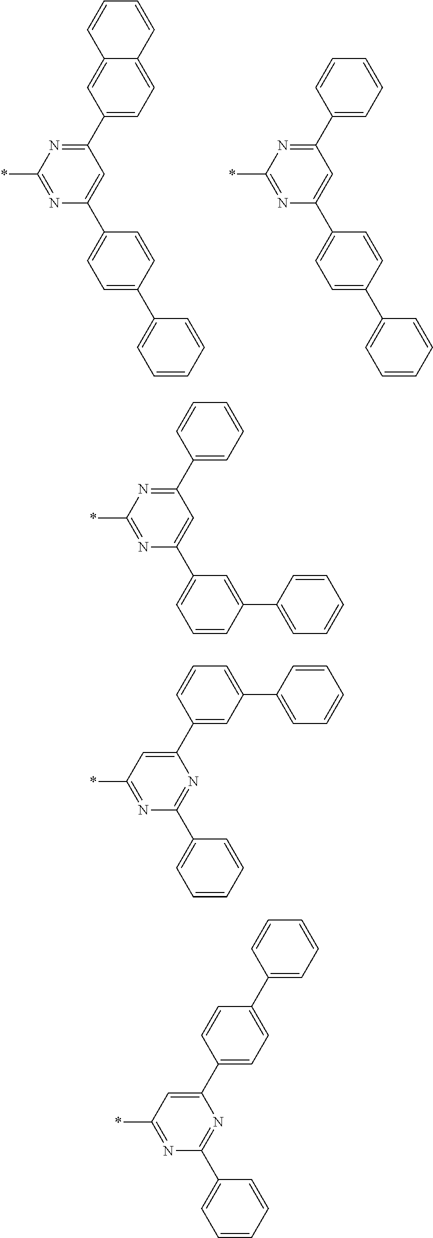

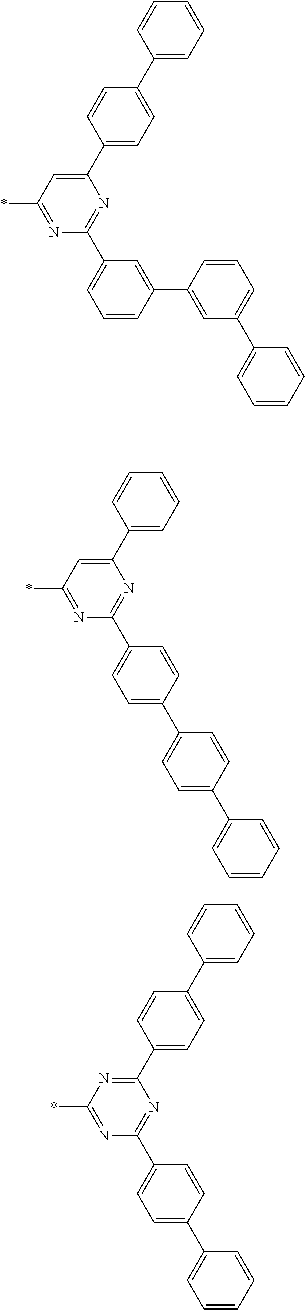

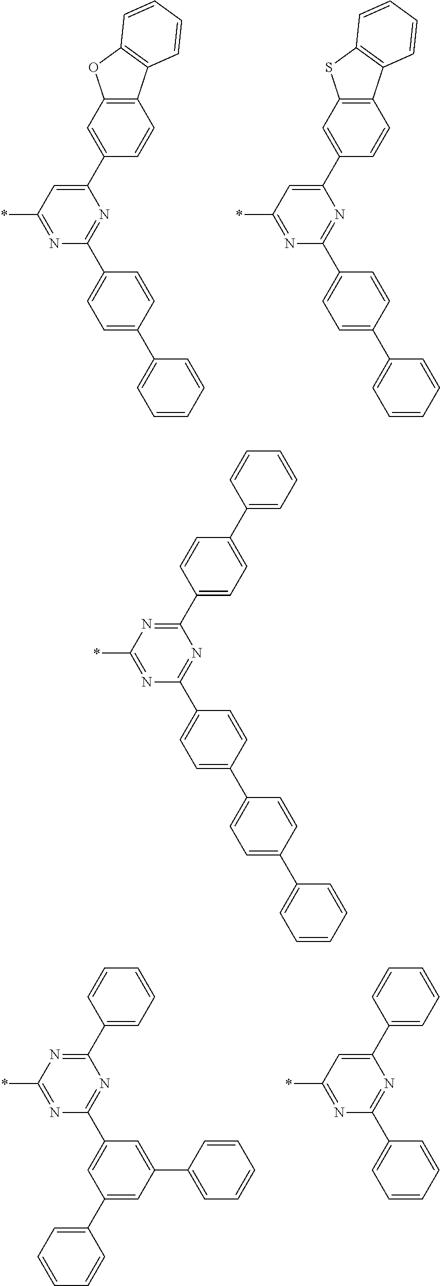 [SMILES: *c1cc(-c2ccc3c(c2)oc2ccccc23)nc(-c2ccc(-c3ccccc3)cc2)n1 ; *c1cc(-c2ccc3c(c2)sc2ccccc23)nc(-c2ccc(-c3ccccc3)cc2)n1 ; *c1nc(-c2ccc(-c3ccccc3)cc2)nc(-c2ccc(-c3ccc(-c4ccccc4)cc3)cc2)n1 ; *c1nc(-c2ccccc2)nc(-c2cc(-c3ccccc3)cc(-c3ccccc3)c2)n1 ; *c1cc(-c2ccccc2)nc(-c2ccccc2)n1]

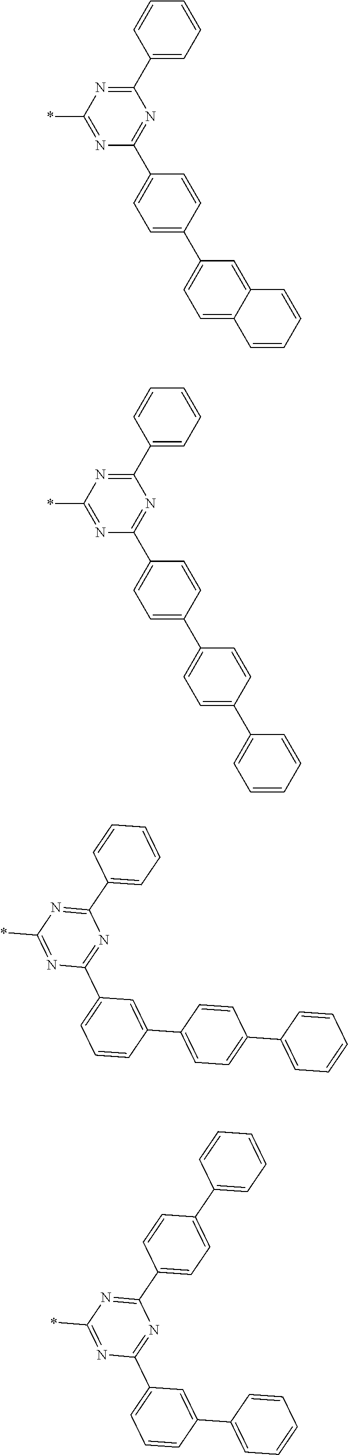 [SMILES: *c1nc(-c2ccccc2)nc(-c2ccc(-c3ccc4ccccc4c3)cc2)n1 ; *c1nc(-c2ccccc2)nc(-c2ccc(-c3ccc(-c4ccccc4)cc3)cc2)n1 ; *c1nc(-c2ccccc2)nc(-c2cccc(-c3ccc(-c4ccccc4)cc3)c2)n1 ; *c1nc(-c2ccc(-c3ccccc3)cc2)nc(-c2cccc(-c3ccccc3)c2)n1]

In Group II, * is a linking point with L³.

In the most specific example embodiment of the present invention, the first host may be represented by Chemical Formula 1-I-1 or Chemical Formula 1-I-2, wherein R⁴ and R⁵ may be for example independently hydrogen, deuterium, a cyano group, a phenyl group, or biphenyl group, Ar¹ of Chemical Formula 1-I-1 may be for example a substituted or unsubstituted phenyl group, a substituted or unsubstituted biphenyl group, a substituted or unsubstituted naphthyl group or a substituted or unsubstituted terphenyl group, and X² of Chemical Formula 1-I-2 may be O or S, and R^{c}, R^{d} and R^{e} may independently be hydrogen, deuterium, a cyano group or phenyl group.

On the other hand, Chemical Formula 1-I may be represented by one of Chemical Formula 1-I A, Chemical Formula 1-I B, Chemical Formula 1-I C, and Chemical Formula 1-I D according to a substitution position of a dibenzofuranyl group (or dibenzothiophenyl group).

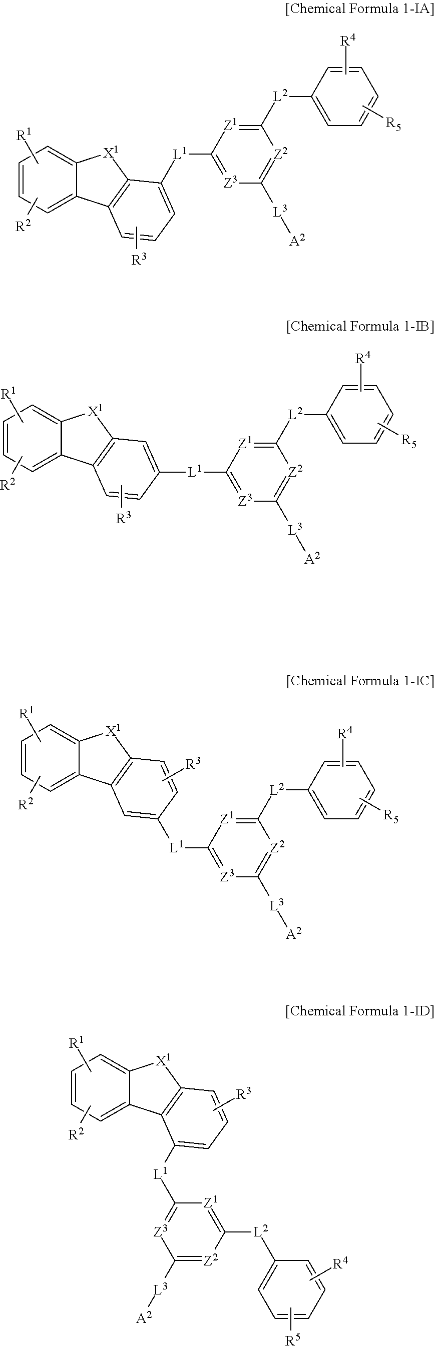

In Chemical Formula 1-I A to Chemical Formula 1-I D, definitions of X¹, Z¹ to Z³, L¹ to L³, R¹ to R⁵ and A² are the same as described above.

In a specific example embodiment of the present invention, Chemical Formula 1-I may be represented by Chemical Formula 1-I B, and in more specific example embodiment, above Chemical Formula 1-I B may be represented by one of Chemical Formula 1-I B-1 to Chemical Formula 1-I B-3.

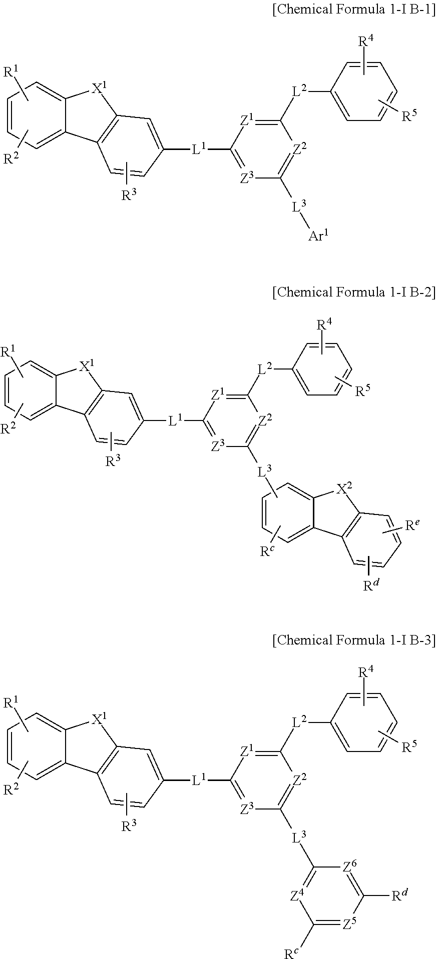

In Chemical Formula 1-I B-1 to Chemical Formula 1-I B-3, X¹ and X², definitions of Z¹ to Z⁶, L¹ to L³, Ar¹, R^{c}, R^{d}, R^{e} and R¹ to R⁵ are the same as described above.

In a more specific example embodiment of the present invention, Chemical Formula 1-I B-1 or Chemical Formula 1-I B-2 are more preferable.

In a specific example embodiment the present invention, R^{c} and R^{d} of Chemical Formula 1-I B-3 may be independently a substituted or unsubstituted C6 to C20 aryl group, and more specifically a phenyl group, a biphenyl group, a naphthyl group, or a terphenyl group. In the most specific example embodiment of the present invention, L¹ to L³ may independently be a single bond or a substituted or unsubstituted phenylene group, a substituted or unsubstituted biphenylene group, a substituted or unsubstituted terphenylene group, or a substituted or unsubstituted naphthylenylene group, and may be for example selected from linking groups of Group III.

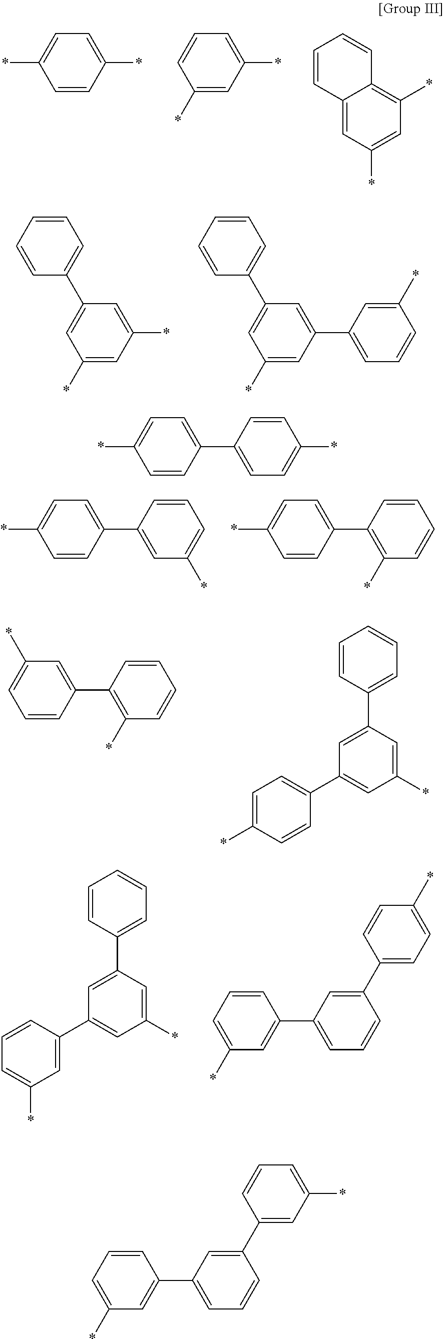

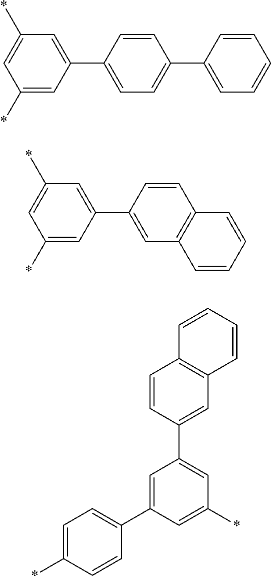

In Group III, * is a linking point.

In a specific example embodiment of the present invention, L¹ to L³ may independently be a single bond or an unsubstituted phenylene group. More specifically, L¹ may be a single bond or an unsubstituted phenylene group, and preferably a single bond. In addition, in a specific example embodiment of the present invention, Chemical Formula 1-I-1 may be represented by Chemical Formula 1-I-1a or Chemical Formula 1-I-1b,

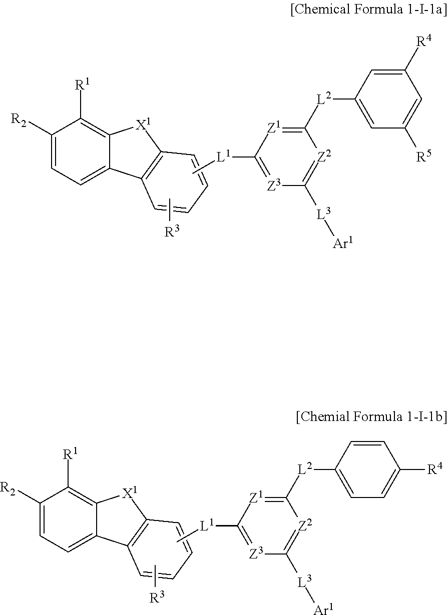

Chemical Formula 1-I-2 may be represented by Chemical Formula 1-I-2a,

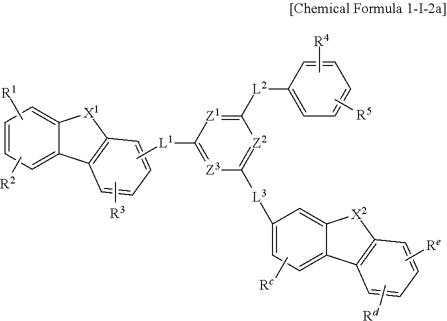

Chemical Formula 1-I-3 may be represented by one of Chemical Formula 1-I-3a, Chemical Formula 1-I-3b, Chemical Formula 1-I-3c, Chemical Formula 1-I-3d, Chemical Formula 1-I-3e, and Chemical Formula 1-I-3f.

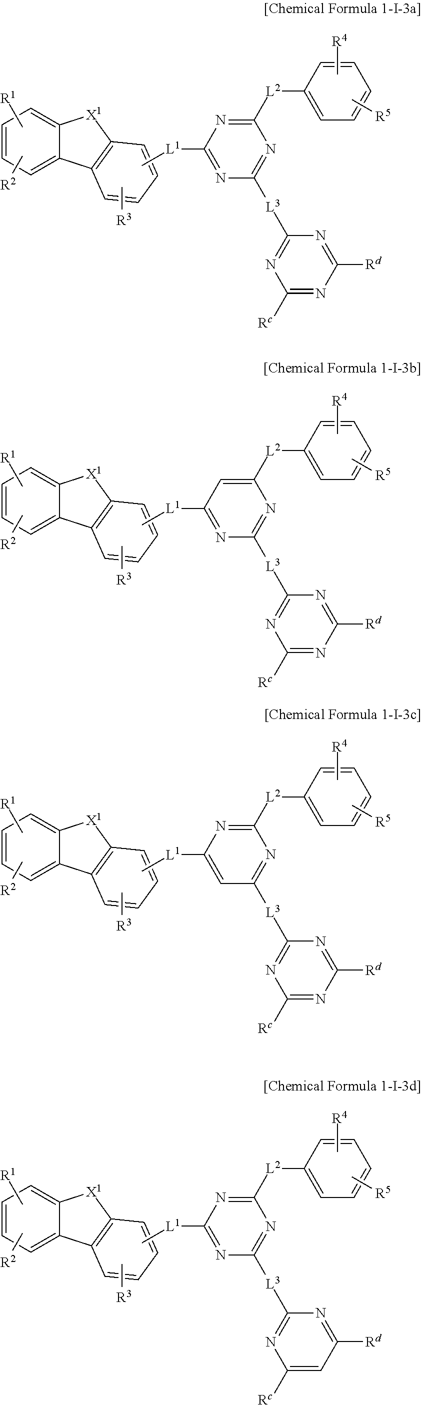

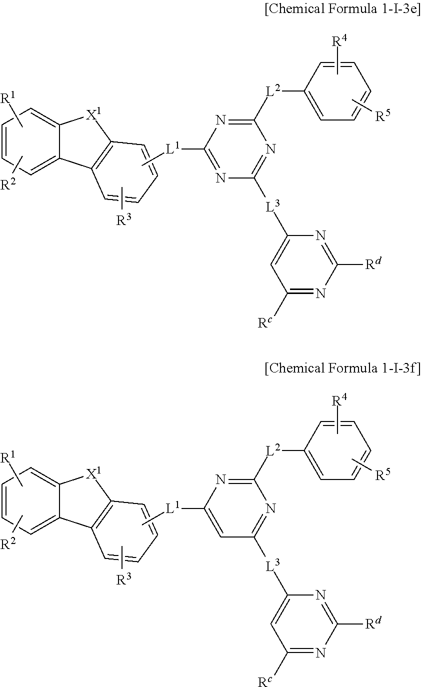

In Chemical Formula 1-I-1a, Chemical Formula 1-I-1b, Chemical Formula 1-I-2a, and Chemical Formula 1-I-3a to Chemical Formula 1-I-3f, definitions of X¹, L¹ to L³, R^{c}, R^{d}, R^{e} and R¹ to R⁵ are the same as described above.

For example, R¹ to R³ of Chemical Formula 1-I-1a, Chemical Formula 1-I-1b, Chemical Formula 1-I-2a, and Chemical Formula 1-I-3a to Chemical Formula 1-I-3f may independently be hydrogen, deuterium, a phenyl group, or a biphenyl group and R⁴ and R⁵ may independently be hydrogen, deuterium, a phenyl group, a biphenyl group, or a terphenyl group, and preferably R¹ to R³ may be all hydrogen and R⁴ and R⁵ are independently hydrogen, a phenyl group, or a biphenyl group.

In addition, a nitrogen-containing hexagonal ring consisting of Z¹ to Z³ of Chemical Formula 1 may be a pyrimidinyl group or a triazinyl group, and more preferably a triazinyl group.

In a specific example embodiment of the present invention, the first host may be for example represented by Chemical Formula 1-I-1 or Chemical Formula 1-I-2, and preferably represented by Chemical Formula 1-I-1a, above Chemical Formula 1-I-1b and Chemical Formula 1-I-2a.

In addition, the first host may be for example represented by Chemical Formula 1-I B, and may be preferably represented by Chemical Formula 1-I B-1 or Chemical Formula 1-I B-2.

The first host may be for example selected from compounds of Group 1, but is not limited thereto.

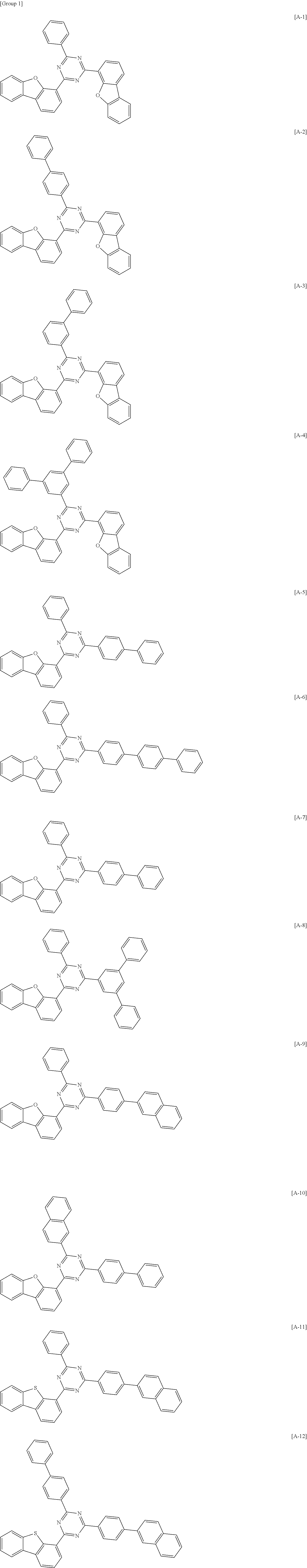

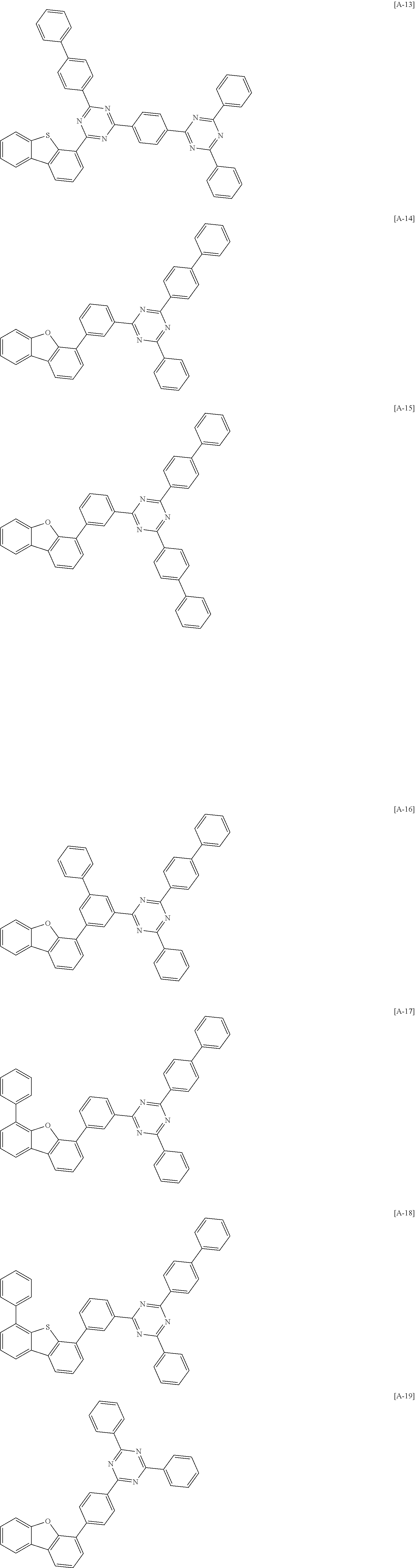

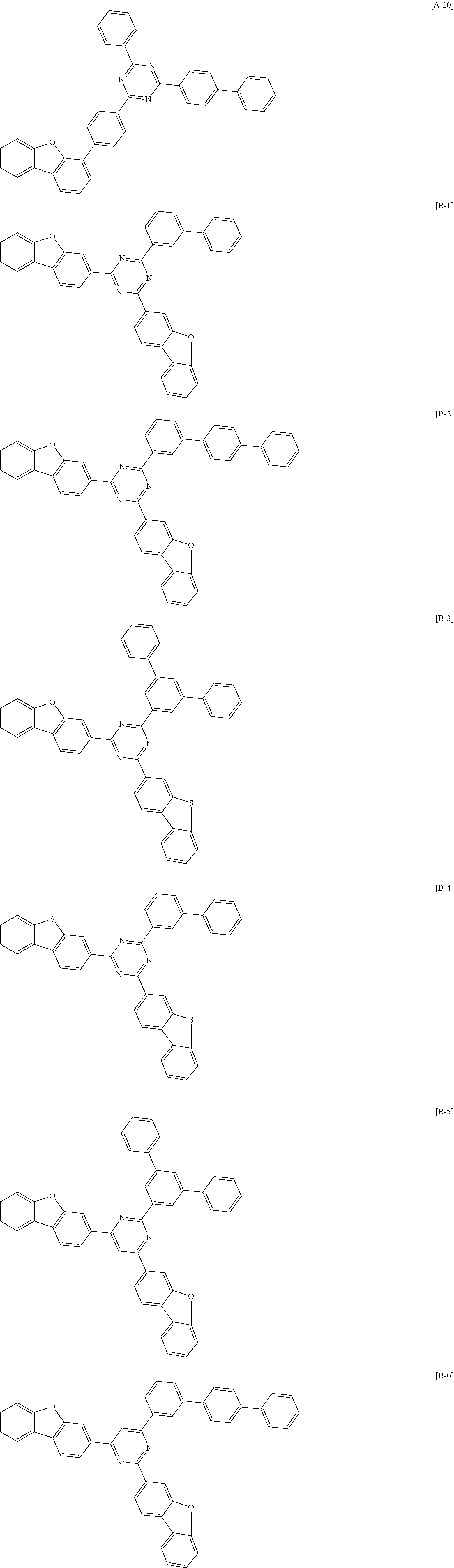

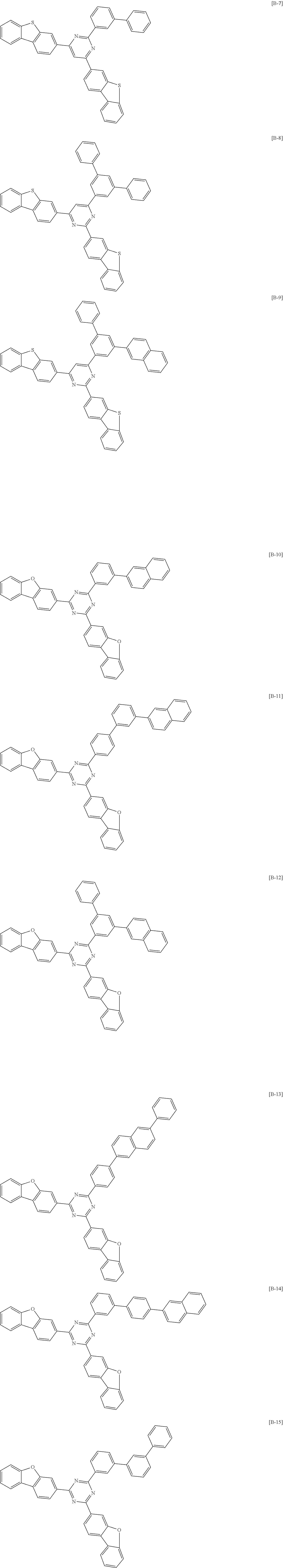

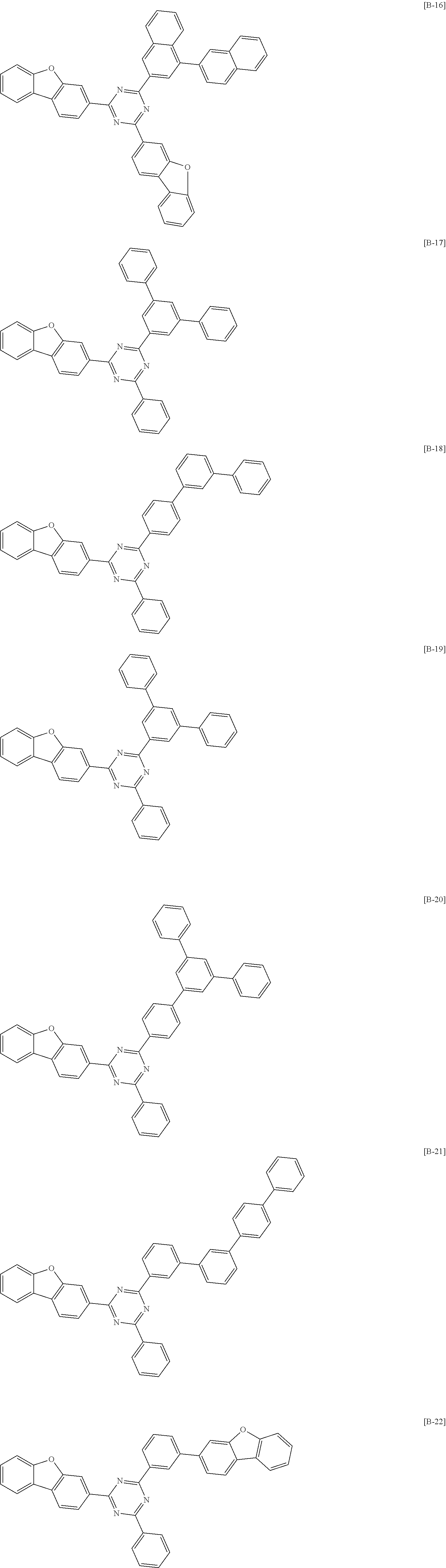

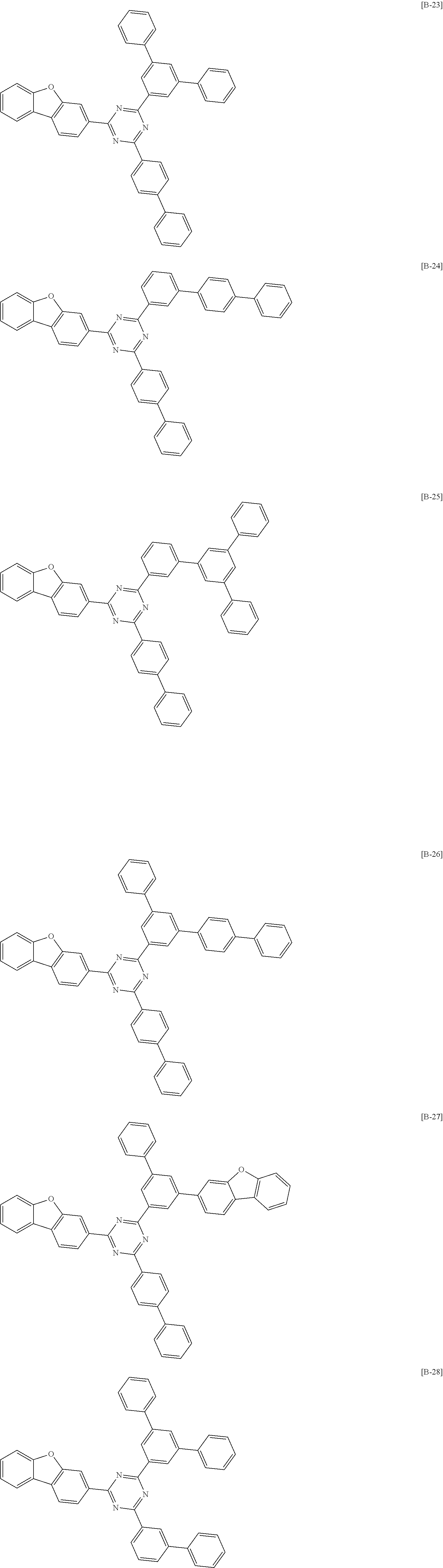

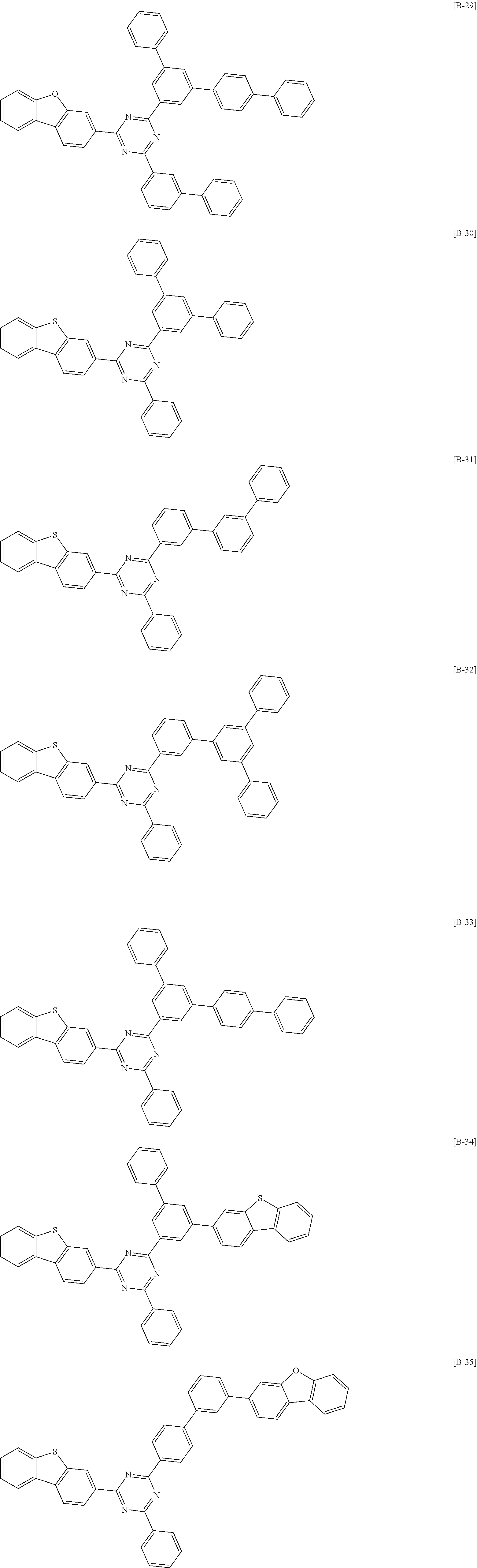

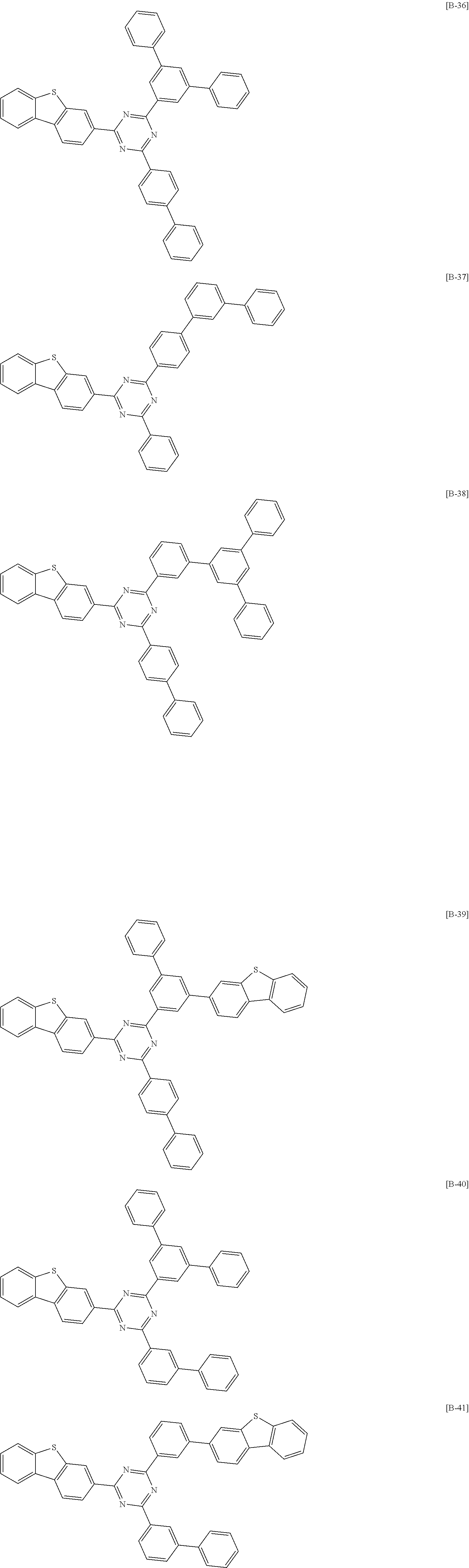

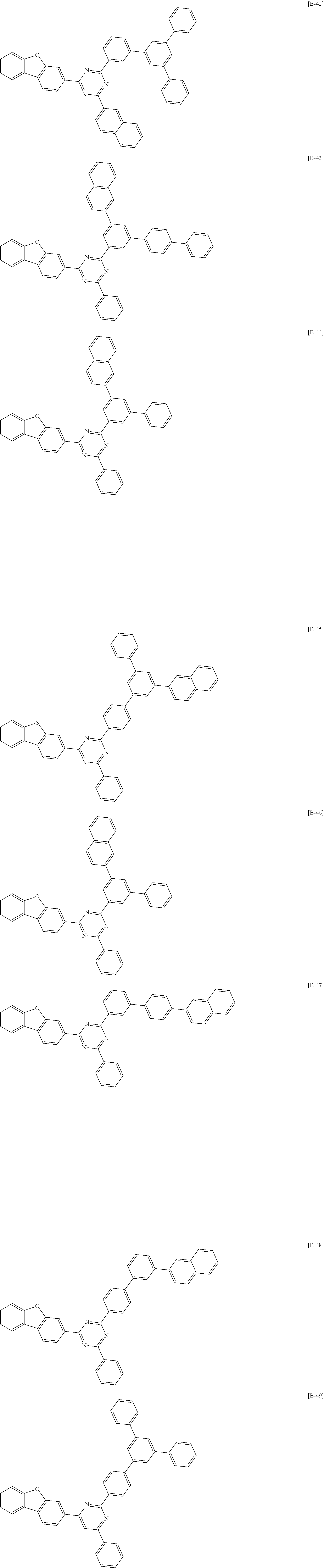

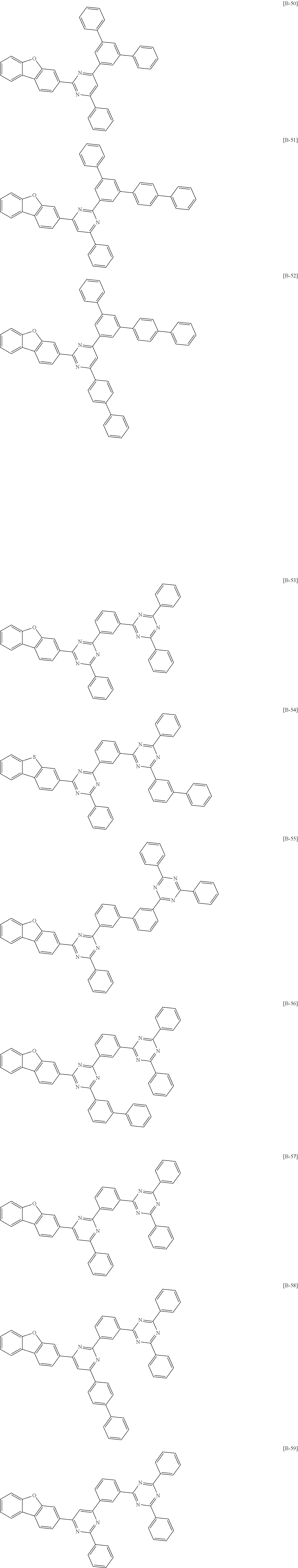

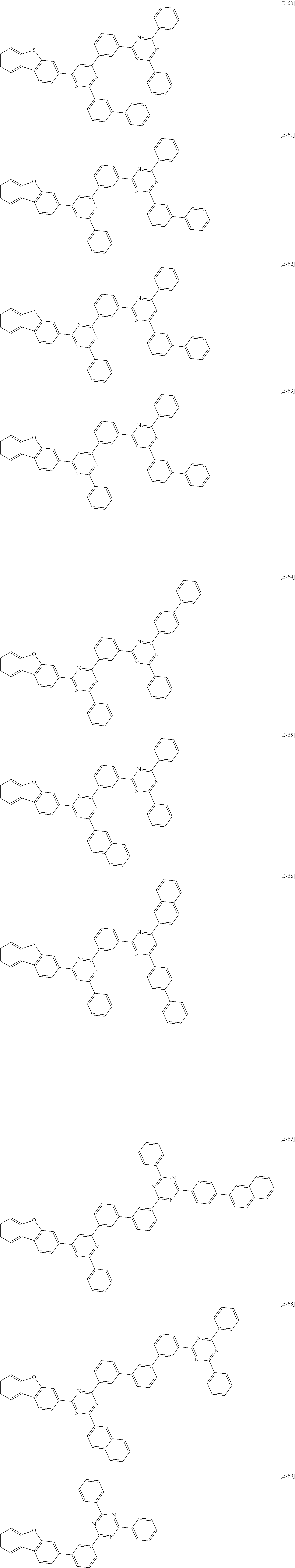

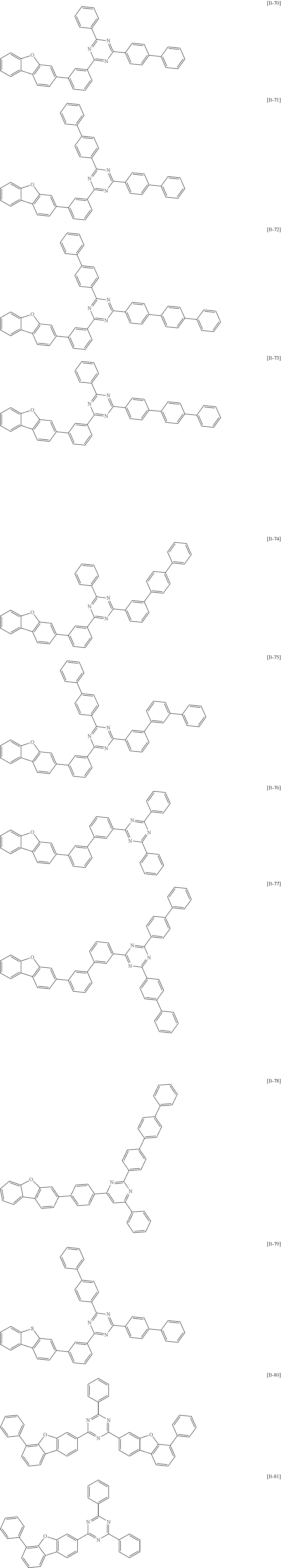

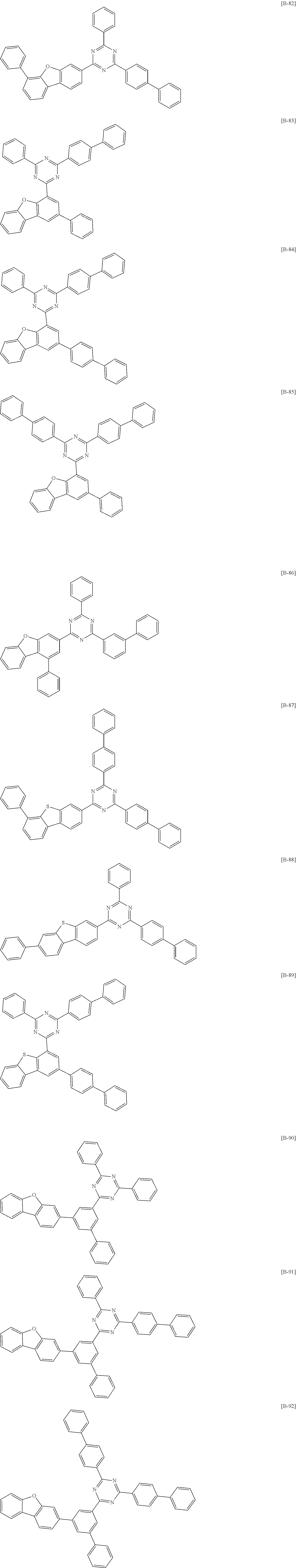

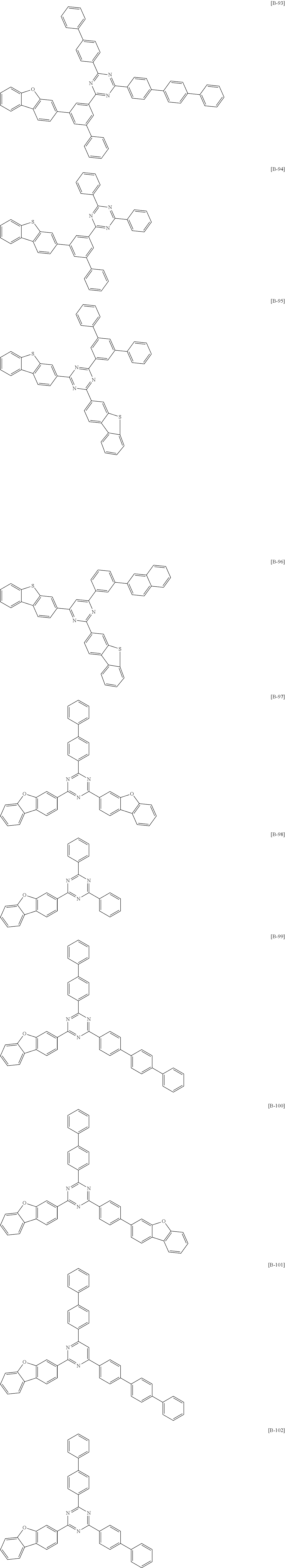

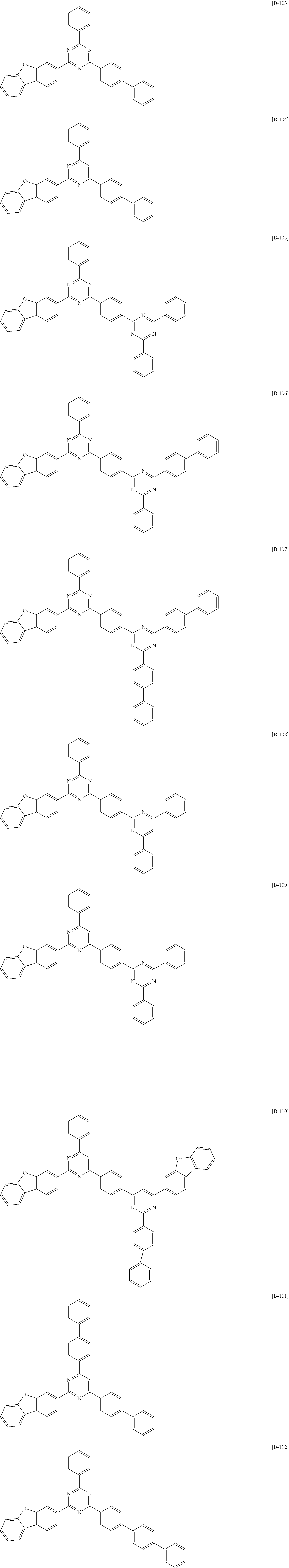

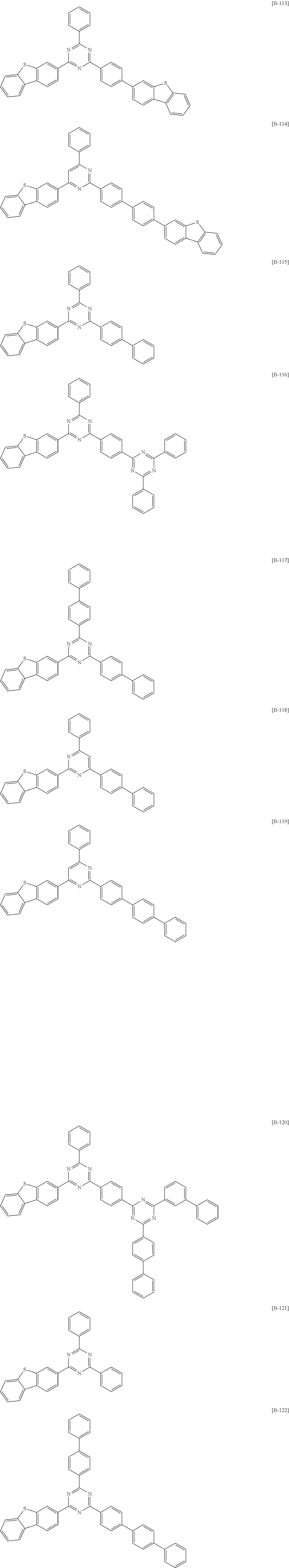

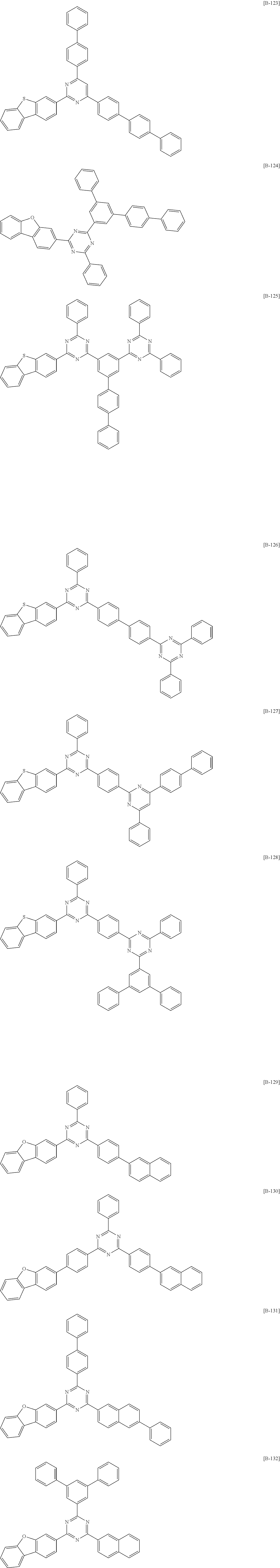

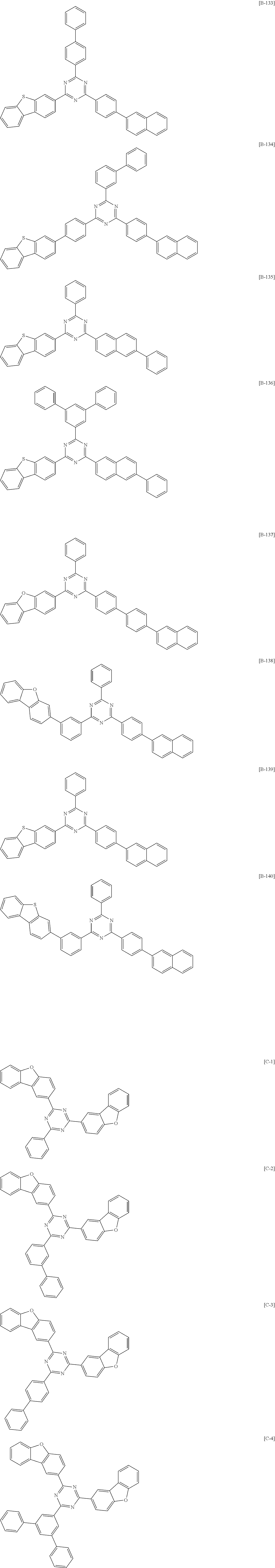

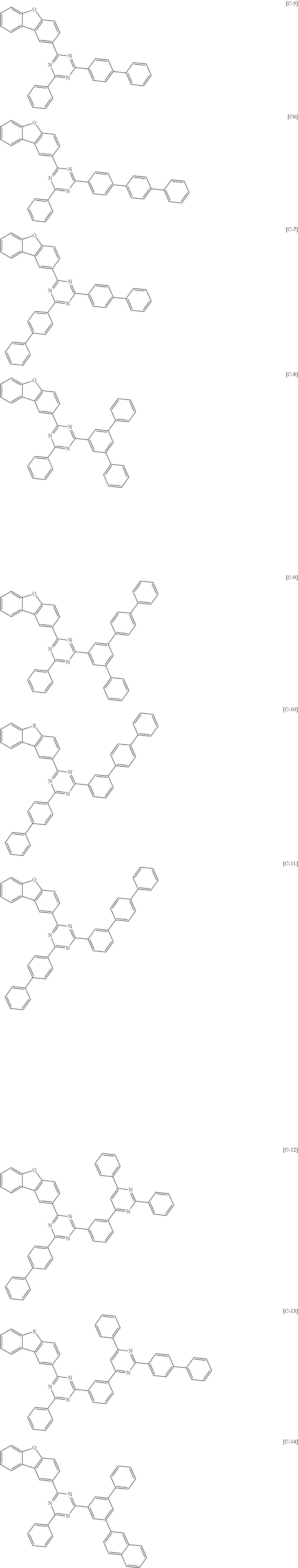

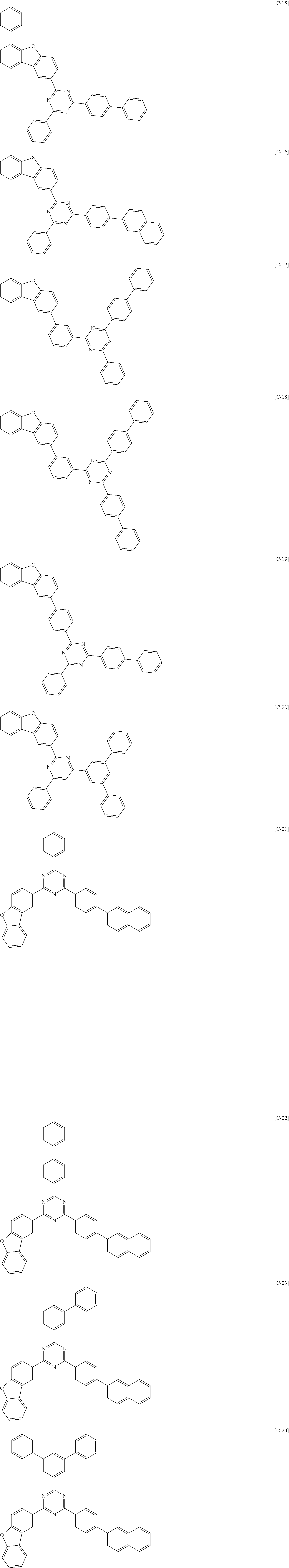

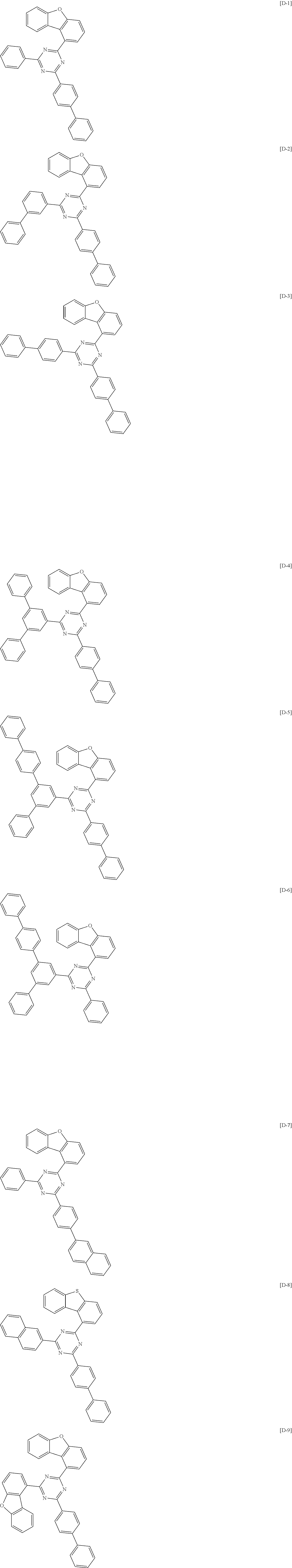

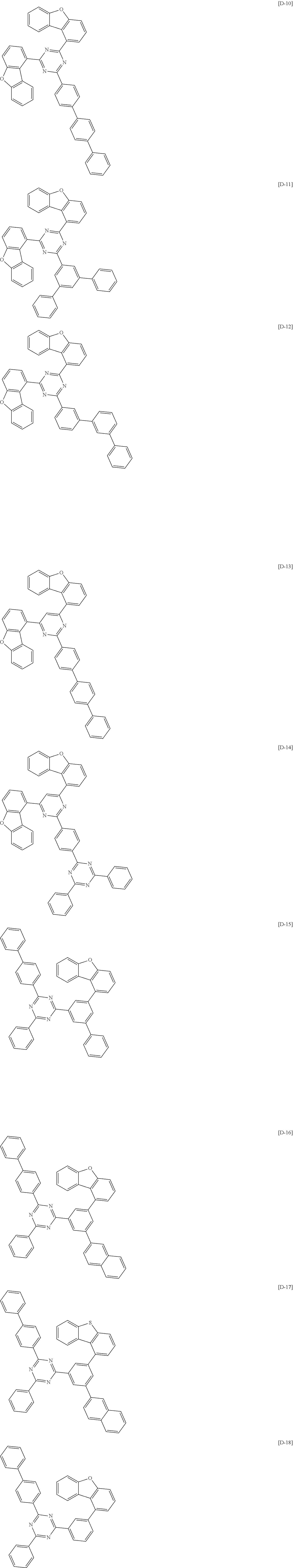

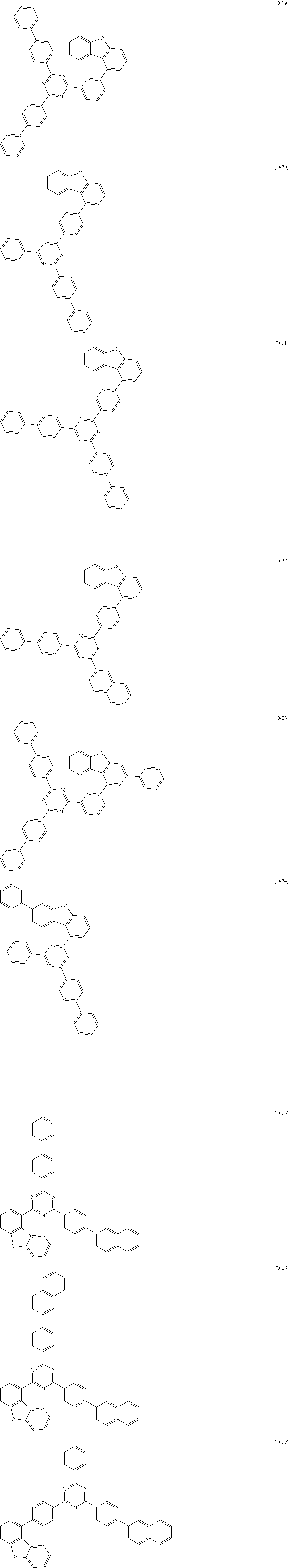

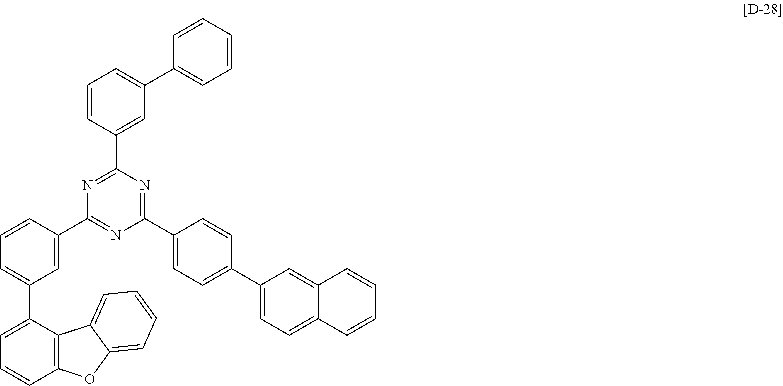 [SMILES: c1ccc(-c2cccc(-c3nc(-c4ccc(-c5ccc6ccccc6c5)cc4)nc(-c4cccc(-c5cccc6oc7ccccc7c56)c4)n3)c2)cc1]

In an example embodiment of the present invention, the second host may be for example represented by one of Chemical Formula 2A, Chemical Formula 2B, Chemical Formula 2C, Chemical Formula 2D, Chemical Formula 2E and Chemical Formula 2F according to a fusion position of Chemical Formula 2 and Chemical Formula 3.

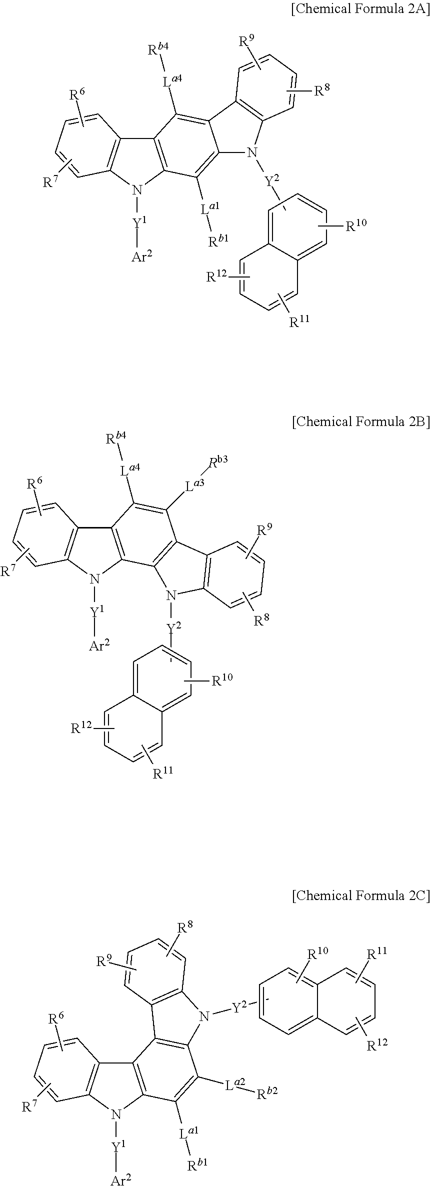

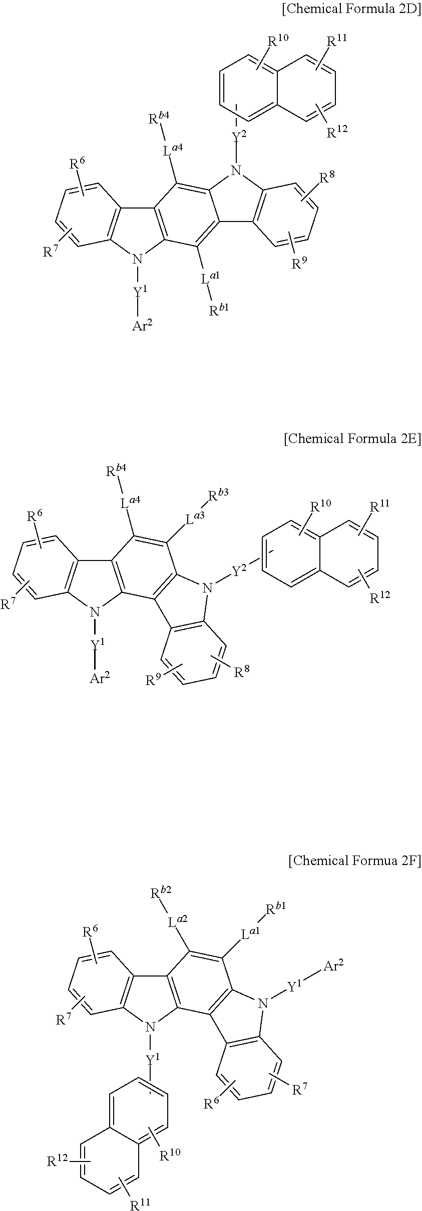

In Chemical Formula 2A to Chemical Formula 2F,

Ar², L^{a}, Y¹ and Y², R^{b} and R⁶ to R¹² are the same as described above, definitions of L^{a1} to L^{a4} are the same as L^{a}, and R^{b1} to R^{b4} are the same as R^{b}.

Ar² may be a substituted or unsubstituted phenyl group, a substituted or unsubstituted biphenyl group, a substituted or unsubstituted naphthyl group, or a substituted or unsubstituted terphenyl group.

In a specific example embodiment of the present invention, the second host may be represented by Chemical Formula 2C and may be for example represented by Chemical Formula 2C-a or Chemical Formula 2C-b according to a substitution point of the naphthyl group.

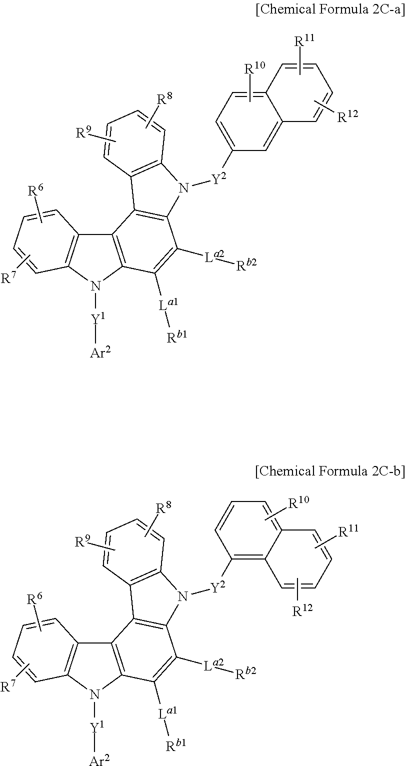

In Chemical Formula 2C-a and Chemical Formula 2C-b, definitions of Ar², L^{a1}, L^{a2}, Y¹, Y², R^{b1}, R^{b2} and R⁶ to R¹² are the same as described above.

In a more specific example embodiment of the present invention, the first host may be represented by Chemical Formula 1-I and the second host may be represented by Chemical Formula 2C-a.

More preferably, the first host may be represented by Chemical Formula 1-I B-1 or Chemical Formula 1-I B-2.

Meanwhile, R^{b1} and R^{b2} and R⁶ to R¹² of Chemical Formula 2C-a may independently be hydrogen, deuterium, a cyano group, a substituted or unsubstituted phenyl group, a substituted or unsubstituted biphenyl group, and

L^{a1} and L^{a2} and Y¹ and Y² may independently be a single bond, a substituted or unsubstituted para-phenylene group, a substituted or unsubstituted meta-phenylene group, or a substituted or unsubstituted biphenylene group.

In an example embodiment of the present invention, R⁶ to R⁹ may independently be hydrogen, deuterium, a cyano group or a phenyl group, or may be all hydrogen.

In an example embodiment of the present invention, R¹⁹ to R¹² may independently be hydrogen, deuterium, a cyano group, or a phenyl group, and more specifically hydrogen or a phenyl group.

In an example embodiment of the present invention, Ar² may be a substituted or unsubstituted phenyl group, a substituted or unsubstituted biphenyl group, a substituted or unsubstituted naphthyl group, or a substituted or unsubstituted terphenyl group. In a more specific example embodiment of the present invention, an additional substituent of Ar² may be deuterium, a cyano group, a phenyl group, or a naphthyl group.

The second host may be for example selected from compounds of Group 2, but is not limited thereto.

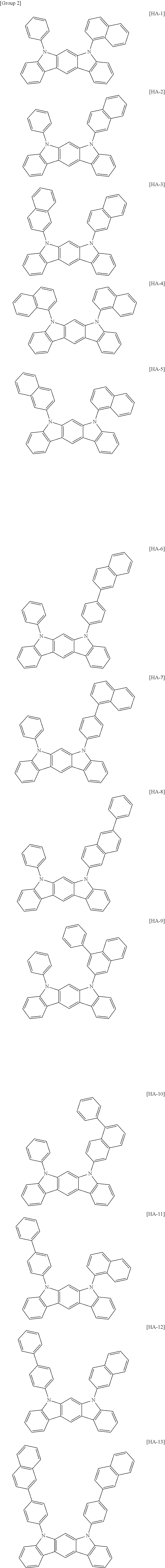

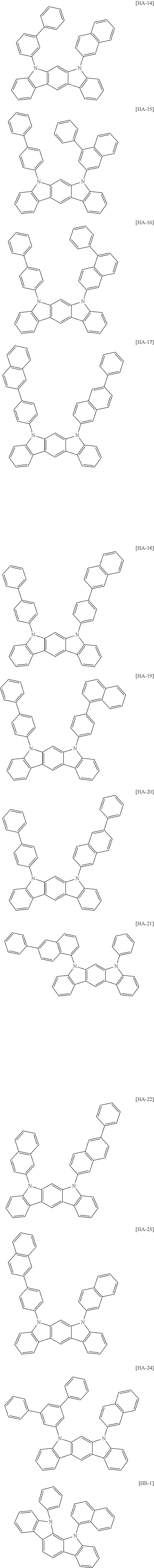

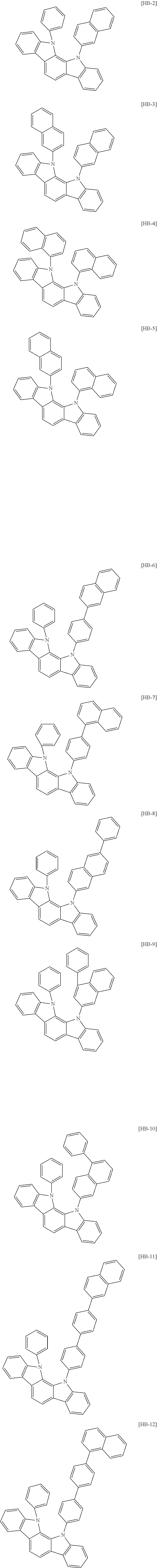

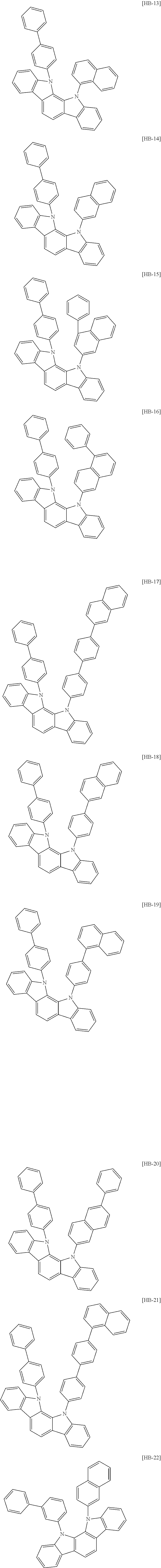

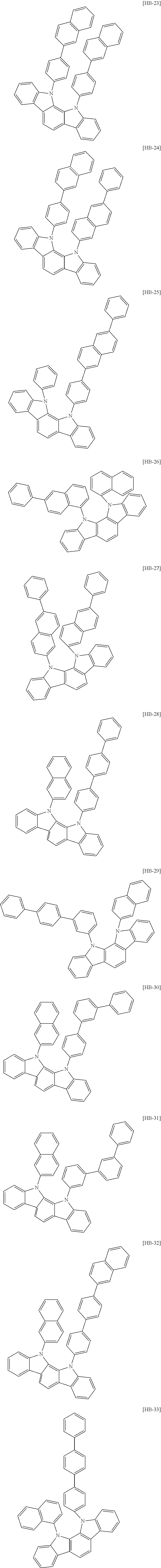

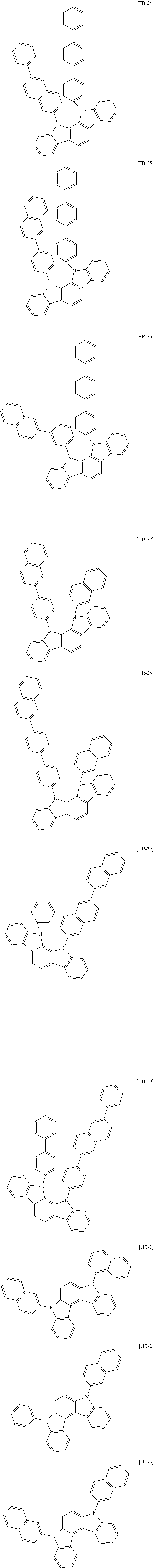

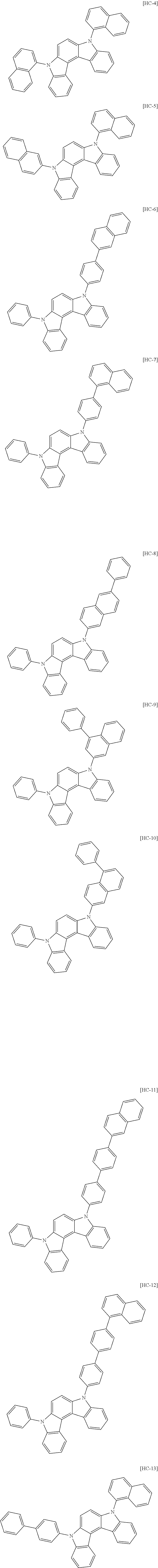

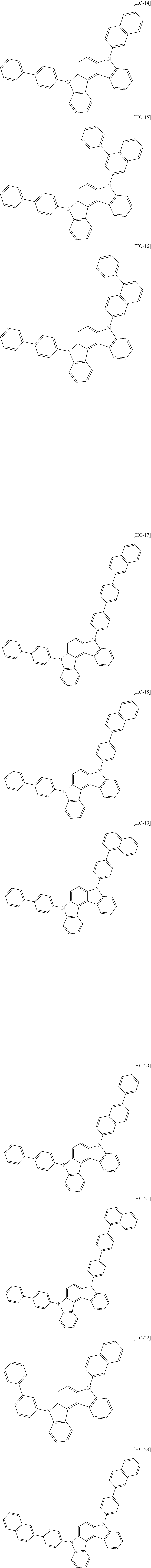

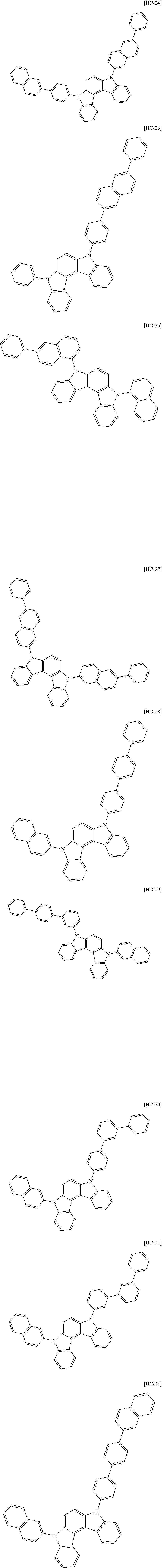

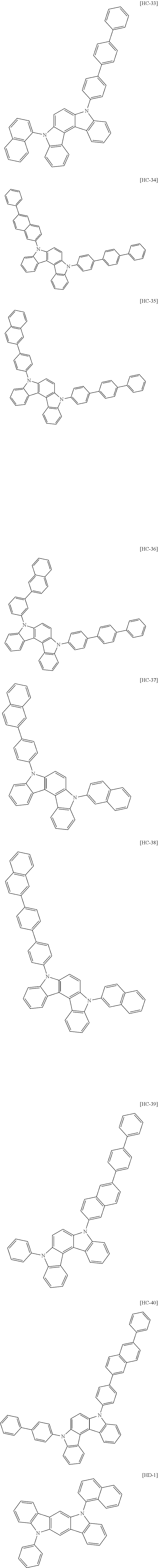

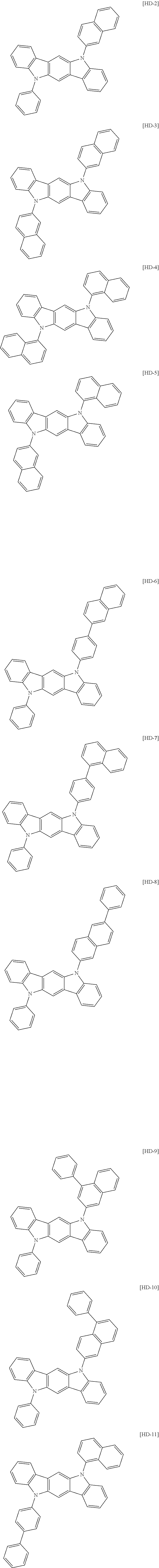

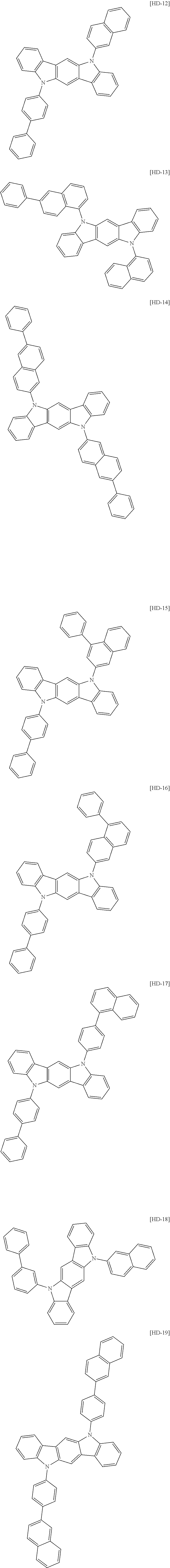

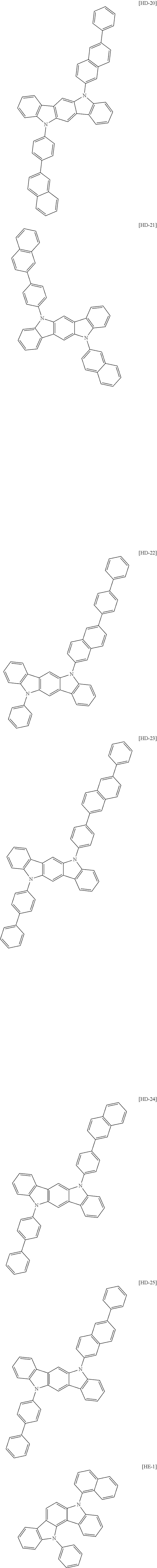

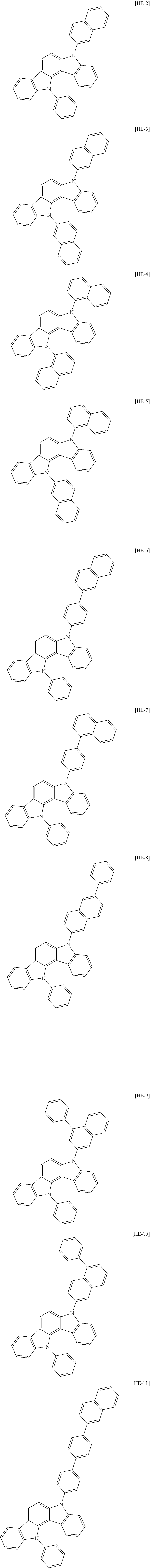

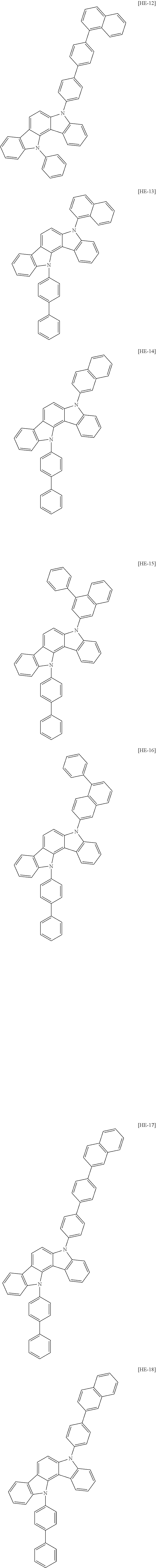

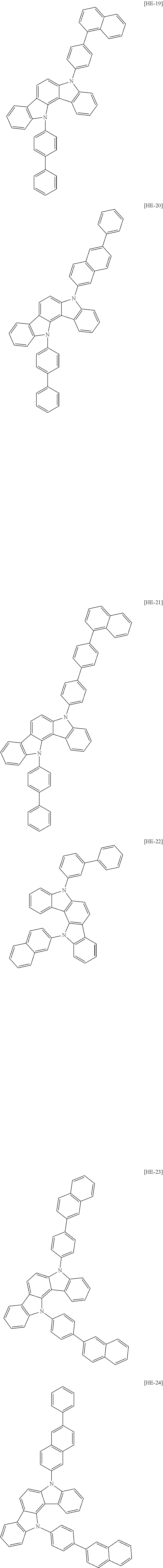

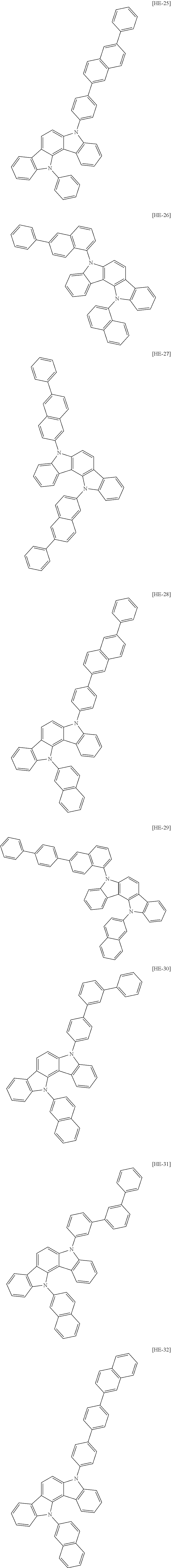

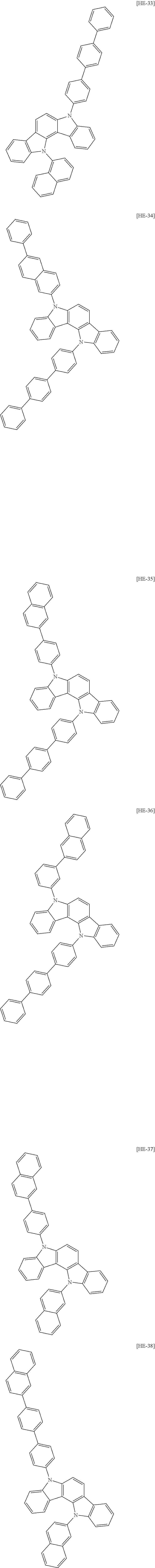

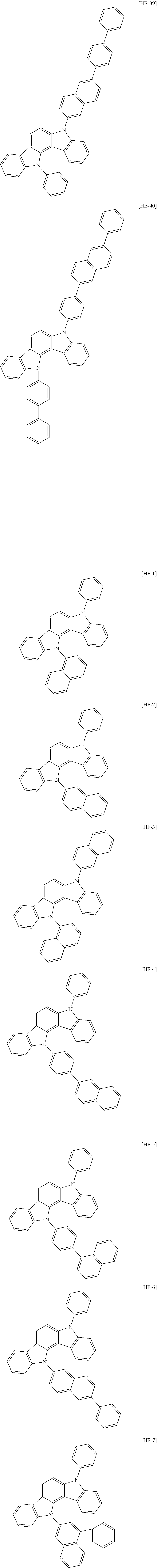

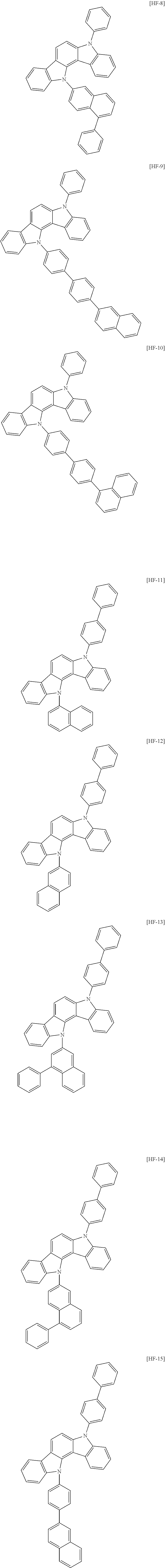

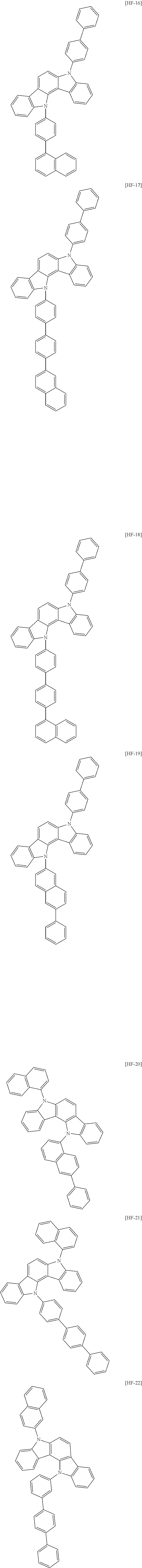

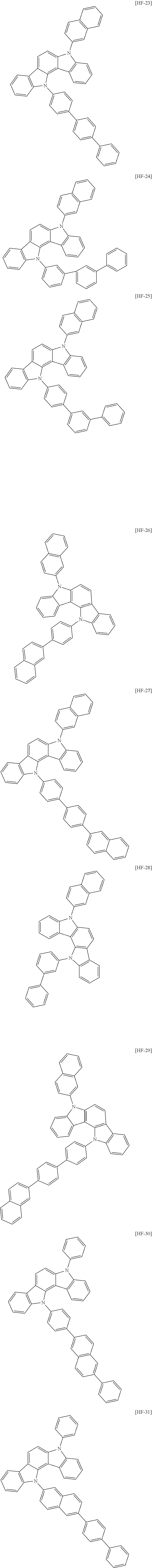

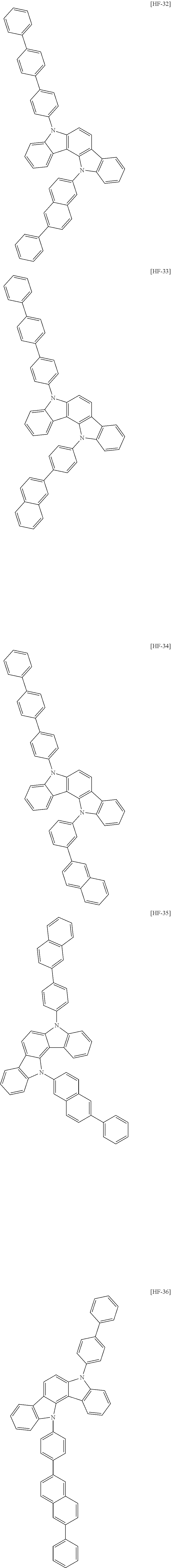

The first host and the second host may be applied as a form of a composition.

That is, the present invention provides a composition for a red phosphorescent host including the first host represented by Chemical Formula 1 and the second host represented by a combination of Chemical Formula 2 and Chemical Formula 3.

In the present invention, the red phosphorescent dopant has a maximum photoluminescence wavelength in a range of 550 nm to 750 nm. In other words, a light emitting device fabricated by applying the composition according to the present invention has a maximum photoluminescence wavelength of a dopant in a long wavelength region beyond a green region.

The organic optoelectronic device of the present invention includes a phosphorescent dopant having a maximum photoluminescence wavelength of 550 nm to 750 nm. In other words, the organic optoelectronic device of the present invention includes a phosphorescent dopant having a maximum photoluminescence wavelength beyond a green region. For example, the maximum photoluminescence wavelength may be in a range of about 560 nm to about 750 nm, which may indicate a reddish region, for example, about 570 nm to about 720 nm, about 580 nm to about 700 nm, about 590 nm to about 700 nm, about 600 nm to about 700 nm, or the like.

The phosphorescent dopant having the maximum photoluminescence wavelength of 550 nm to 750 nm may be an iridium (Ir) complex or a platinum (Pt) complex, and the platinum (Pt) complex may be for example represented by Chemical Formula 4-1. In addition, the iridium (Ir) complex may be may be for example represented by Chemical Formula 4-2.

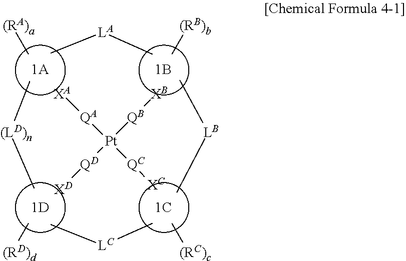

In Chemical Formula 4-1,

X^{A}, X^{B}, X^{C}, and X^{D} are elements that form unsaturated rings with each of 1A, 1B, 1C, and 1D, and independently C or N,

1A, 1B, 1C, and 1D are independently a substituted or unsubstituted C6 to C30 aryl group, or a substituted or unsubstituted C2 to C30 heterocyclic group,

L^{A}, L^{B}, L^{C}, L^{D}, Q^{A}, Q^{B}, Q^{C} and Q^{D} are independently a single bond, O, S, a substituted or unsubstituted C1 to C30 alkylene group, a substituted or unsubstituted C2 to C30 alkenylene group, a substituted or unsubstituted C6 to C30 arylene group, or a substituted or unsubstituted C2 to C30 heteroarylene group,

R^{A}, R^{B}, R^{C}, and R^{D} are independently hydrogen, deuterium, a cyano group, a halogen, silane group, phosphine group, amine group, a substituted or unsubstituted C1 to C30 alkyl group, a substituted or unsubstituted C6 to C30 aryl group, or a substituted or unsubstituted C2 to C30 heteroaryl group,

R^{A}, R^{B}, R^{C}, and R^{D} are independently present or adjacent groups are linked with each other to form a ring,

n is one of integers of 0 to 5, and

a, b, c, and d are independently one of integers of 0 to 3.

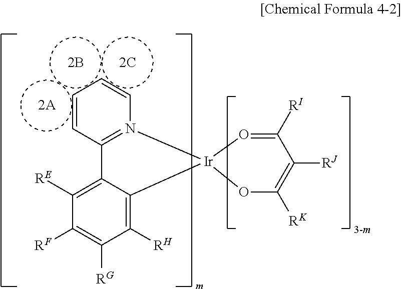

In Chemical Formula 4-2,

2A, 2B, and 2C are independently a substituted or unsubstituted benzene ring,

at least one of 2A, 2B, and 2C forms a fused ring with an adjacent complex compound,

R^{E}, R^{F}, R^{G}, R^{H}, R^{I}, R^{J}, and R^{K} are independently hydrogen, deuterium, a cyano group, a halogen, silane group, phosphine group, amine group, a substituted or unsubstituted C1 to C10 alkyl group, a substituted or unsubstituted C6 to C30 aryl group, or a substituted or unsubstituted C2 to C30 heteroaryl group,

R^{E}, R^{F}, R^{G}, R^{H}, R^{I}, R^{J}, and R^{K} are independently present or adjacent groups are linked with each other to form a ring, and

m is one of integers of 1 to 3.

In an example embodiment of the present invention, the platinum (Pt) complex may be represented by Chemical Formula 4-1a or Chemical Formula 4-1b.

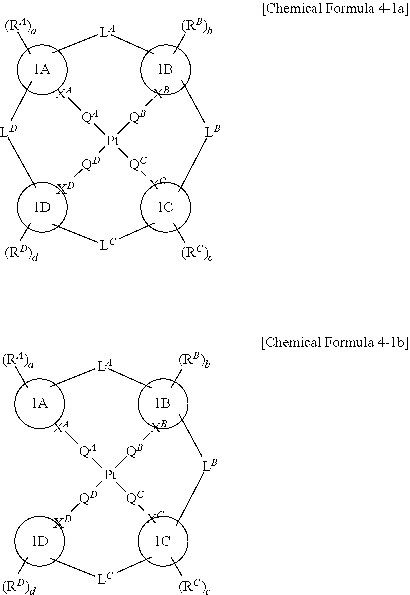

In Chemical Formula 4-1a and Chemical Formula 4-1b, definitions of X^{A}, X^{B}, X^{C}, X^{D}, 1A, 1B, 1C, 1D, L^{A}, L^{B}, L^{C}, L^{D}, Q^{A}, Q^{B}, Q^{C}, Q^{D}, R^{A}, R^{B}, R^{C}, R^{D}, a, b, c, and d are the same as described above.

In a specific example embodiment of the present invention, 1A, 1B, 1C and 1D may independently be a substituted or unsubstituted C6 to C20 aryl group, or a substituted or unsubstituted C2 to C20 heterocyclic group, more specifically, a substituted or unsubstituted phenyl group, a substituted or unsubstituted naphthyl group, a substituted or unsubstituted anthracenyl group, a substituted or unsubstituted phenanthrenyl group, a substituted or unsubstituted triphenylenyl group, a substituted or unsubstituted pyridinyl group, a substituted or unsubstituted benzimidazolyl group, a substituted or unsubstituted benzothiazole group, a substituted or unsubstituted benzoxazole group, a substituted or unsubstituted pyrrolyl group, a substituted or unsubstituted pyrazolyl group, a substituted or unsubstituted imidazolyl group, a substituted or unsubstituted oxazolyl group, and may be for example selected from groups of Group IV, and groups of Group IV may be further substituted.

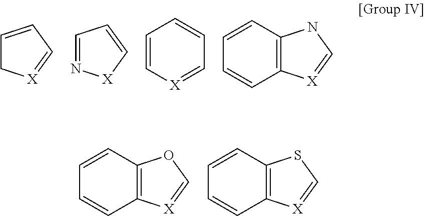

In Group IV, X is an element that forms an unsaturated ring with each of 1A, 1B, 10, and 1D, and independently C or N. Additional substituents may be deuterium, a cyano group, a halogen, a C1 to C10 alkyl group, or a C1 to C10 fluoroalkyl group.

More preferably, 1A, 1B, 10, and 1D may be a substituted or unsubstituted phenyl group, a substituted or unsubstituted pyridinyl group, a substituted or unsubstituted benzimidazolyl group, a substituted or unsubstituted benzothiazole group, a substituted or unsubstituted pyrrolyl group, or a substituted or unsubstituted pyrazolyl group.

In a specific example embodiment of the present invention, when a, b, c and d are 2 or greater, each of substituents R^{A}, R^{B}, R^{C} and R^{D} may be the same or different.

Meanwhile, specific examples of the present invention include structures where adjacent groups of R^{A}, R^{B}, R^{C}, and R^{D} are fused to form a ring. For example, Compound 3-5 or Compound 3-8 of Group 3 may be exemplified.

In an example embodiment of the present invention, the iridium (Ir) complex may be represented by Chemical Formula 4-2a, or Chemical Formula 4-2b.

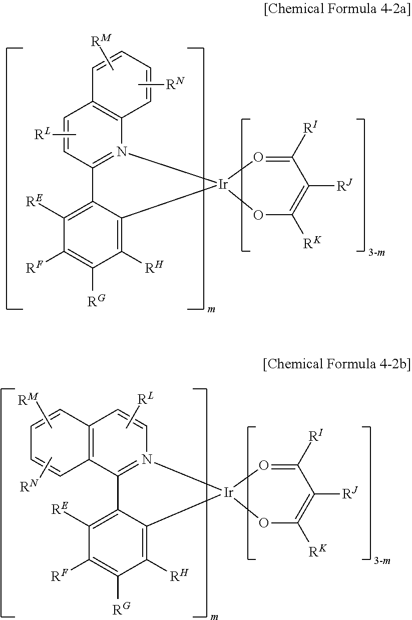

In Chemical Formula 4-2a and Chemical Formula 4-2b, definitions of R^{E}, R^{F}, R^{G}, R^{H}, R^{I}, R^{J}, R^{K}, and m are the same as described above, and definitions of R^{L}, R^{M}, and R^{N} are the same as definitions of R^{E}, R^{F}, R^{G}, R^{H}, R^{I}, R^{J}, and R^{K}.

In a specific example embodiment of the present invention, R^{E}, R^{F}, R^{G}, R^{H}, R^{I}, R^{K}, R^{L}, R^{M}, and R^{N} may be hydrogen, deuterium, a cyano group, a halogen, a C1 to C10 alkyl group, or a C1 to C10 fluoroalkyl group.

Meanwhile, specific examples of the present invention include structures where adjacent groups of R^{E}, R^{F}, R^{G}, and R^{H} are fused to form a ring. For example, Compound 4-12 of Group 3 may be exemplified.

The phosphorescent dopant may be for example selected from compounds of Group 3, but is not limited thereto.

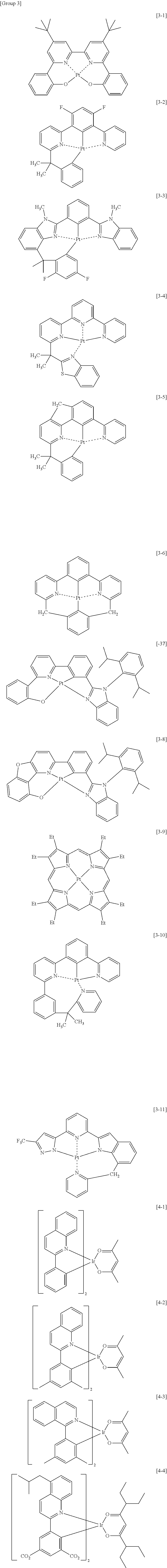

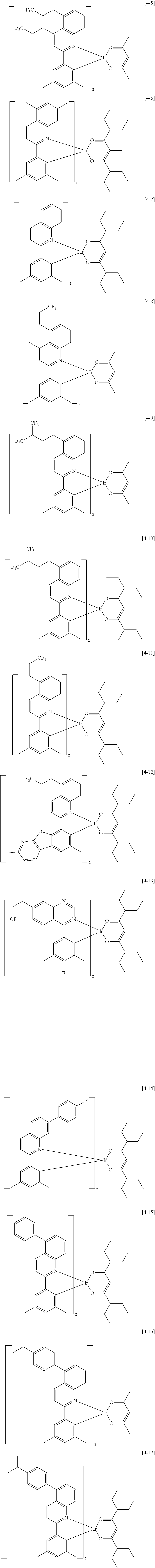

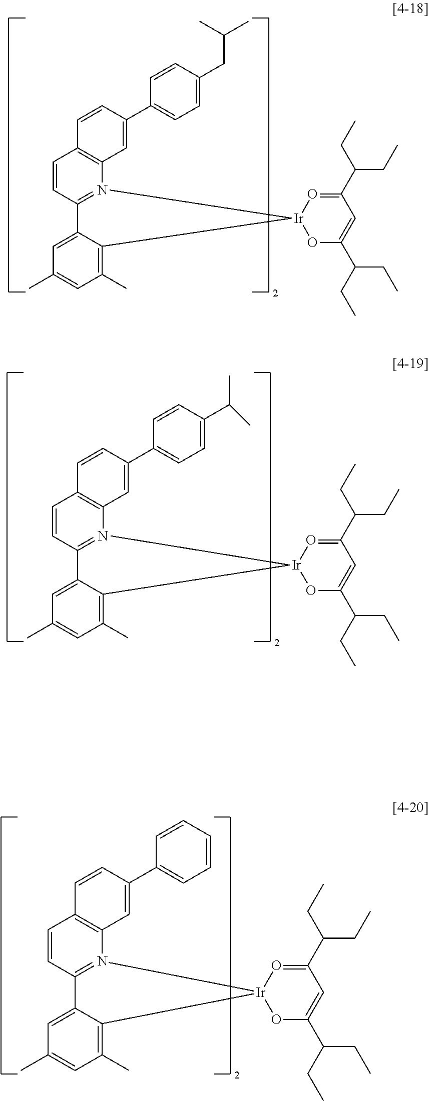

In the most specific example embodiment of the present invention, the first host may be represented by Chemical Formula 1-I B-1 or Chemical Formula 1-I B-2, the second host may be represented by Chemical Formula 2C-a, and the phosphorescent dopant may be represented by Chemical Formula 4-2a.

More specifically, the first host and the second host may be included in a weight ratio of 1:9 to 5:5, 2:8 to 5:5, or 3:7 to 5:5, and the phosphorescent dopant may be included in an amount of 0.1 to 50 wt % based on 100 wt % of the composition of the first host and the second host. In addition, the first host and the second host may be included in a weight ratio of 3:7 to 5:5 and the phosphorescent dopant may be included in an amount of 0.1 to 10 wt % based on 100 wt % of the composition of the first host and the second host. More specifically, the first host and second host may be included in a weight ratio of 3:7 or 5:5 and the phosphorescent dopant may be included in an amount of 0.5 to 10 wt % based on 100 wt % of the composition of the first host and the second host.

A composition for a red phosphorescent host according to another embodiment may include the first host represented by Chemical Formula 1 and the second host represented by a combination of Chemical Formula 2 and Chemical Formula 3.

In an example embodiment of the present invention, the first host may be represented by Chemical Formula 1-I and the second host may be represented by Chemical Formula 2C.

In a specific example embodiment of the present invention, the first host may be represented by Chemical Formula 1-I B-1 or Chemical Formula 1-I B-2, wherein Ar¹ of Chemical Formula 1-I B-1 may be a substituted or unsubstituted phenyl group, a substituted or unsubstituted biphenyl group, a substituted or unsubstituted naphthyl group, a substituted or unsubstituted terphenyl group, or a substituted or unsubstituted quaterphenyl group. Definitions of other substituents are the same as described above.

The organic light emitting diode may be applied to an organic light emitting diode (OLED) display.

### MODE FOR INVENTION

Hereinafter, the embodiments are illustrated in more detail with reference to examples. These examples, however, are not in any sense to be interpreted as limiting the scope of the invention.

Hereinafter, starting materials and reactants used in Examples and Synthesis Examples were purchased from Sigma-Aldrich Co. Ltd. or TCI Inc. as far as there in no particular comment or were synthesized by known methods.

The compound as one specific examples of the present invention was synthesized through the following steps.

(Preparation of First Host)

### Synthesis Example 1: Synthesis of Compound B-1

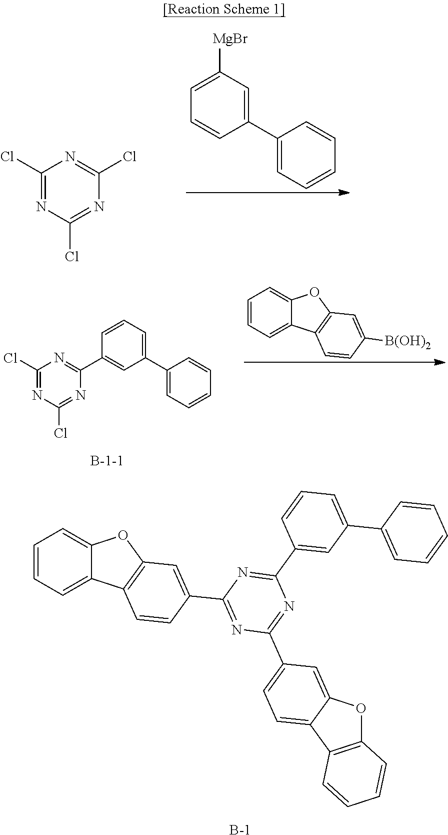

### a) Synthesis of Intermediate B-1-1

15 g (81.34 mmol) of cyanuric chloride was dissolved in 200 mL of anhydrous tetrahydrofuran in a 500 mL round-bottomed flask, 1 equivalent of 3-biphenyl magnesium bromide solution (0.5 M tetrahydrofuran) was added thereto in a dropwise fashion at 0° C. under a nitrogen atmosphere, and the mixture was slowly heated up to room temperature. The reaction solution was stirred at room temperature for 1 hour and in 500 mL of ice water to separate layers. After separating an organic layer therefrom, the resultant was treated with anhydrous magnesium sulfate and concentrated. The concentrated residue was recrystallized with tetrahydrofuran and methanol to obtain 17.2 g of Intermediate B-1-1.

### b) Synthesis of Compound B-1

17.2 g (56.9 mmol) of Intermediate B-1-1 were put in 200 mL of tetrahydrofuran and 100 mL of distilled water in a 500 mL round-bottomed flask, 2 equivalents of dibenzofuran-3-boronic acid (Cas: 395087-89-5), 0.03 equivalents of tetrakistriphenylphosphine palladium, and 2 equivalents of potassium carbonate were added thereto, and the mixture was heated and refluxed under a nitrogen atmosphere. After 18 hours, the reaction solution was cooled down, and a solid precipitated therein was filtered and washed with 500 mL of water. The solid was recrystallized with 500 mL of monochlorobenzene to obtain 12.87 g of Compound B-1.

LC/MS calculated for: C₃₉H₂₃N₃O₂ Exact Mass: 565.1790 found for: 566.18 [M+H]

### Synthesis Example 2: Synthesis of Compound B-3

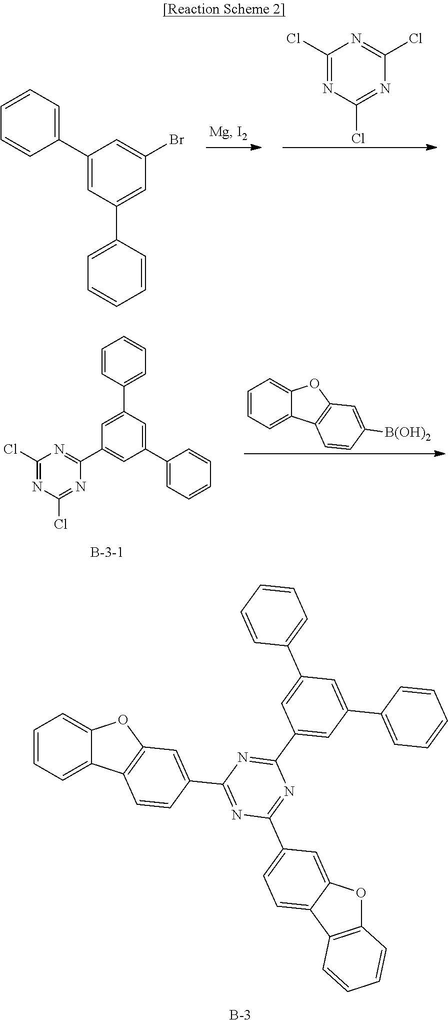

### a) Synthesis of Intermediate B-3-1

7.86 g (323 mmol) of magnesium and 1.64 g (6.46 mmol) of iodine were put in 0.1 L of tetrahydrofuran (THF) under a nitrogen environment, the mixture was stirred for 30 minutes, and 100 g (323 mmol) of 1-bromo-3,5-diphenylbenzene dissolved in 0.3 L of THF was slowly added thereto in a dropwise fashion at 0° C. over 30 minutes. This obtained mixed solution was slowly added in a dropwise fashion to a solution prepared by dissolving 64.5 g (350 mmol) of cyanuric chloride in 0.5 L of THF at 0° C. over 30 minutes. When a reaction was complete, water was added to the reaction solution, and an extract was obtained by using dichloromethane (DCM), treated with anhydrous MgSO₄ to remove moisture, and then, filtered and concentrated under a reduced pressure. This obtained residue was separated and purified through flash column chromatography to obtain Intermediate B-3-1 (79.4 g, 65%).
b) Synthesis of Compound B-3

Compound B-3 was synthesized by using Intermediate B-3-1 according to the same method as b) of Synthesis Example 1.

LC/MS calculated for: C₄₅H₂₇N₃O₂ Exact Mass: 641.2103 found for 642.21 [M+H]

### Synthesis Example 3: Synthesis of Compound B-17

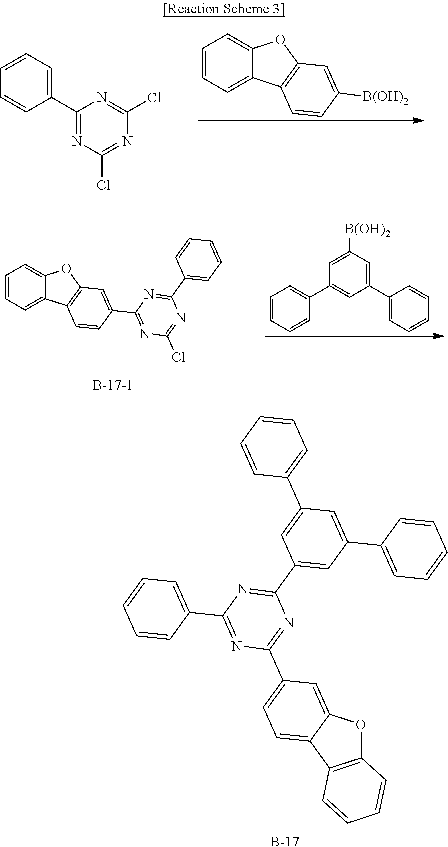

### a) Synthesis of Intermediate B-17-1

4-dichloro-6-phenyltriazine (22.6 g, 100 mmol) was added to 100 mL of tetrahydrofuran, 100 mL of toluene, and 100 mL of distilled water in a 500 mL round-bottomed flask, 0.9 equivalents of dibenzofuran-3-boronic acid (CAS No.: 395087-89-5), 0.03 equivalents of tetrakistriphenylphosphine palladium, and 2 equivalents of potassium carbonate were added thereto, and the mixture was heated and refluxed under a nitrogen atmosphere. After 6 hours, the reaction solution was cooled down, and an organic layer obtained by removing an aqueous layer was dried under a reduced pressure. A solid obtained therefrom was washed with water and hexane and recrystallized with toluene (200 mL) to obtain 21.4 g of Intermediate B-17-1 (a yield of 60%).

### b) Synthesis of Compound B-17

The synthesized Intermediate B-17-1 (56.9 mmol) was added to tetrahydrofuran (200 mL) and distilled water (100 mL) in a 500 mL round-bottomed flask, 1.1 equivalents of 3,5-diphenylbenzeneboronic acid (CAS No.: 128388-54-5), 0.03 equivalents of tetrakistriphenylphosphine palladium, and 2 equivalents of potassium carbonate were added thereto, and the mixture was heated and refluxed under a nitrogen atmosphere. After 18 hours, the reaction solution was cooled down, and a solid precipitated therein was filtered and washed with 500 mL of water. The solid was recrystallized with 500 mL of monochlorobenzene to obtain Compound B-17.

LC/MS calculated for: C₃₉H₂₅N₃O Exact Mass: 555.1998 found for 556.21 [M+H]

### Synthesis Example 4: Synthesis of Compound B-124

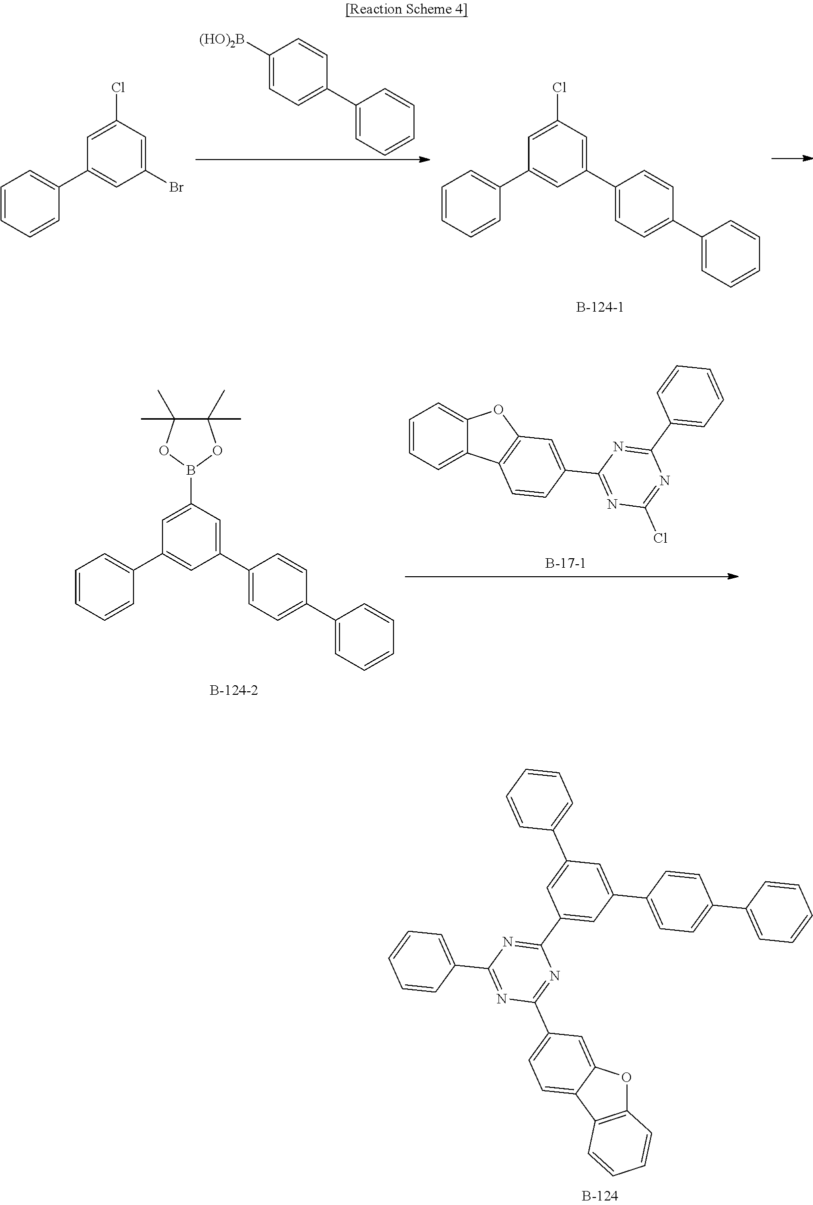 [SMILES: Clc1cc(Br)cc(-c2ccccc2)c1 ; OB(O)c1ccc(-c2ccccc2)cc1 ; Clc1cc(-c2ccccc2)cc(-c2ccc(-c3ccccc3)cc2)c1 ; CC1(C)OB(c2cc(-c3ccccc3)cc(-c3ccc(-c4ccccc4)cc3)c2)OC1(C)C ; Clc1nc(-c2ccccc2)nc(-c2ccc3c(c2)oc2ccccc23)n1 ; c1ccc(-c2ccc(-c3cc(-c4ccccc4)cc(-c4nc(-c5ccccc5)nc(-c5ccc6c(c5)oc5ccccc56)n4)c3)cc2)cc1]

### a) Synthesis of Intermediate B-124-1

Intermediate B-124-1 was synthesized according to the same method as b) of Synthesis Example 1 by using 1-bromo-3-chloro-5-phenylbenzene and 1.1 equivalents of biphenyl-4-boronic acid. Herein, a product was purified through flash column with hexane instead of the recrystallization.

### b) Synthesis of Intermediate B-124-2

30 g (88.02 mmol) of the synthesized Intermediate B-124-1 was added to 250 mL of DMF in a 500 mL round-bottomed flask, 0.05 equivalents of dichlorodiphenylphosphinoferrocene palladium, 1.2 equivalents of bispinacolato diboron, and 2 equivalents of potassium acetate were added thereto, and the mixture was heated and refluxed under a nitrogen atmosphere for 18 hours. The reaction solution was cooled down and then, dropped in 1 L of water to obtain a solid. The solid was dissolved in boiling toluene to treat activated carbon and then, filtered through silica gel and concentrated. The concentrated solid was stirred with a small amount of hexane and then, filtered to obtain 28.5 g of Intermediate B-124-2 (yield 70%).

### c) Synthesis of Compound B-124

Compound B-124 was synthesized according to the same method as b) of Synthesis Example 3 by using Intermediate B-124-2 and Intermediate B-17-1 in each amount of 1.0 equivalent.

LC/MS calculated for: C₄₅H₂₉N₃O Exact Mass: 627.2311 found for 628.22 [M+H]

### Synthesis Example 5: Synthesis of Compound B-23

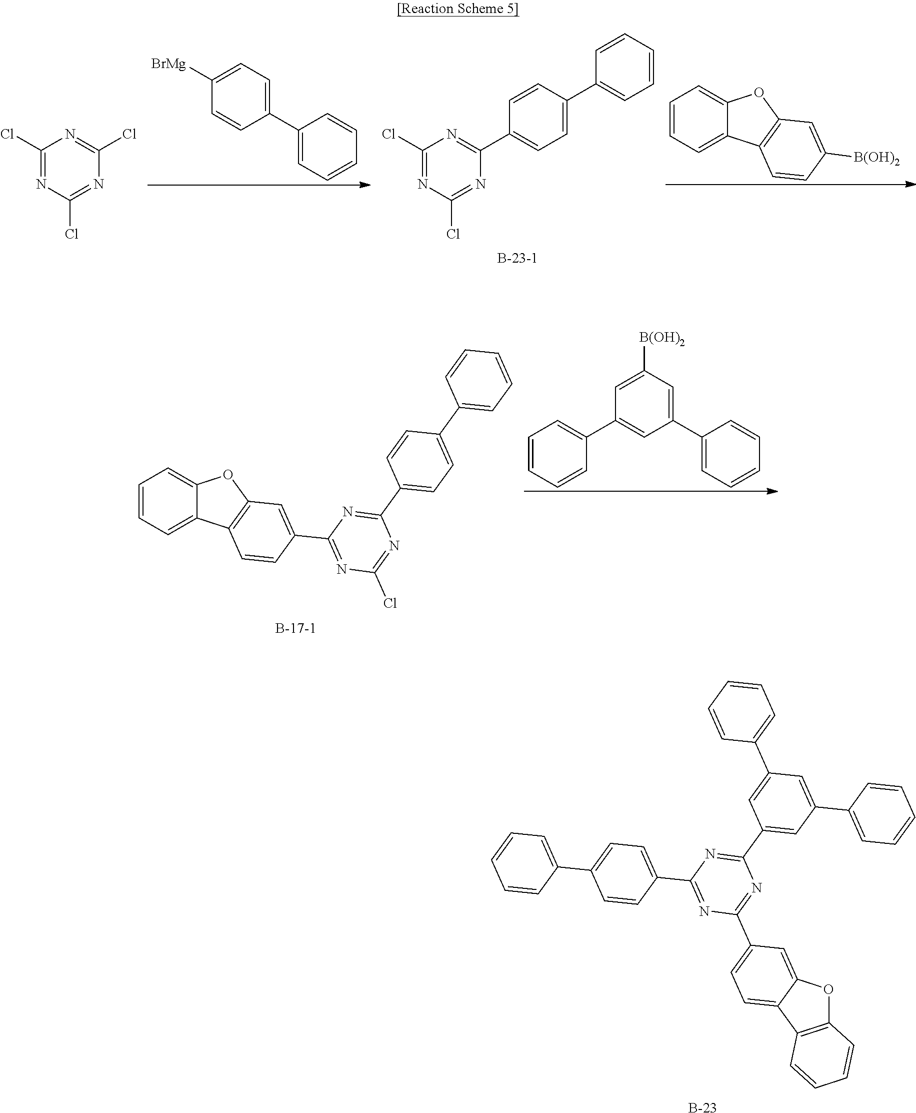

### a) Synthesis of Intermediate B-23-1

Cyanuric chloride (15 g, 81.34 mmol) was dissolved in anhydrous tetrahydrofuran (200 mL) in a 500 mL round-bottomed flask, 1 equivalent of a 4-biphenyl magnesium bromide solution (0.5 M tetrahydrofuran) was added thereto in a dropwise fashion at 0° C. under an nitrogen atmosphere, and the mixture was slowly heated up to room temperature. The mixture was stirred at the same room temperature for 1 hour, and 500 mL of ice water was added thereto to separate layers. An organic layer was separated therefrom and then, treated with anhydrous magnesium sulfate and concentrated. The concentrated residue was recrystallized with tetrahydrofuran and methanol to obtain Intermediate B-23-1 (17.2 g).

### b) Synthesis of Intermediate B-23-2

Intermediate B-23-2 was synthesized according to the same method as a) of Synthesis Example 3 by using Intermediate B-23-1.

### c) Synthesis of Compound B-23

Compound B-23 was synthesized according to the same method as b) of Synthesis Example 3 by using Intermediate B-23-2 and 1.1 equivalents of 3,5-diphenylbenzeneboronic acid.

LC/MS calculated for: C₄₅H₂₉N₃O Exact Mass: 627.2311 found for 628.24 [M+H]

### Synthesis Example 6: Synthesis of Compound B-24

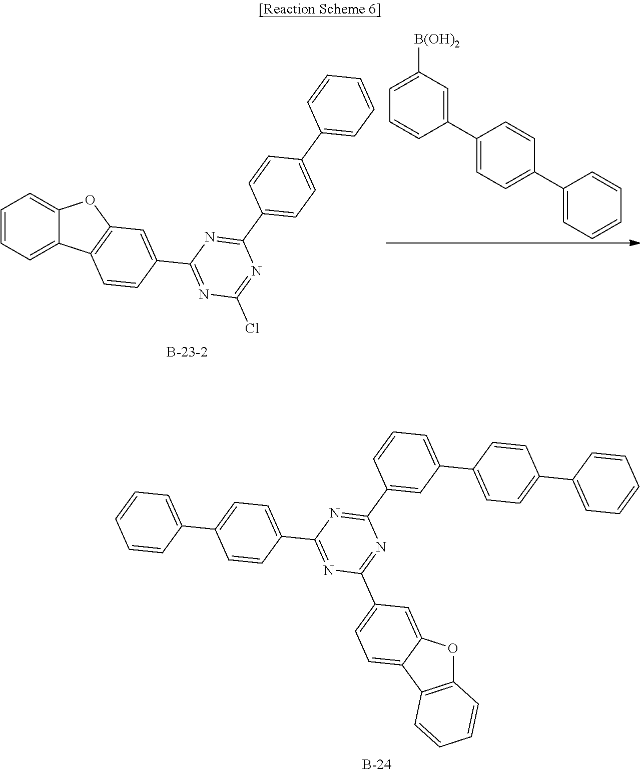

Compound B-24 was synthesized according to the same method as b) of Synthesis Example 3 by using Intermediate B-23-2 and 1.1 equivalents of B-[1,1′:4′,1″-terphenyl]-3-ylboronic acid.

LC/MS calculated for: C₄₅H₂₉N₃O Exact Mass: 627.2311 found for 628.24 [M+H]

### Synthesis Example 7: Synthesis of Compound B-20

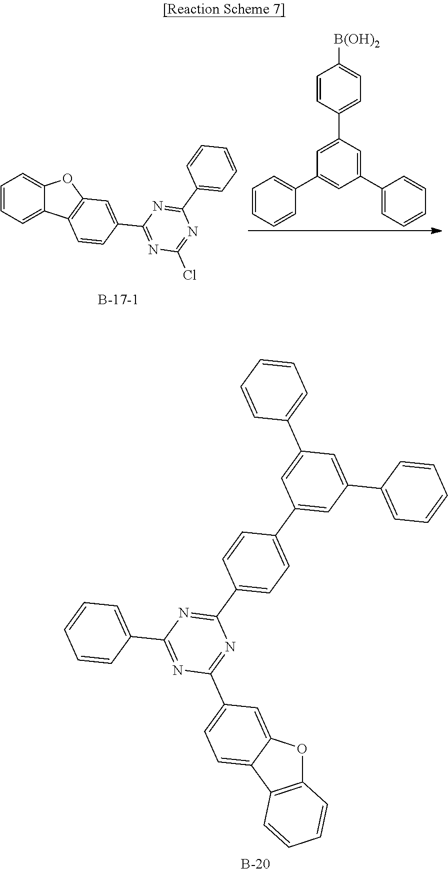

Compound B-20 was synthesized according to the same method as b) of Synthesis Example 3 by using Intermediate B-17-1 and 1.1 equivalents of (5′-phenyl[1,1′:3′,1″-terphenyl]-4-yl)-boronic acid (CAS No.: 491612-72-7).

LC/MS calculated for: C₄₅H₂₉N₃O Exact Mass: 627.2311 found for 628.24 [M+H]

### Synthesis Example 8: Synthesis of Compound B-71

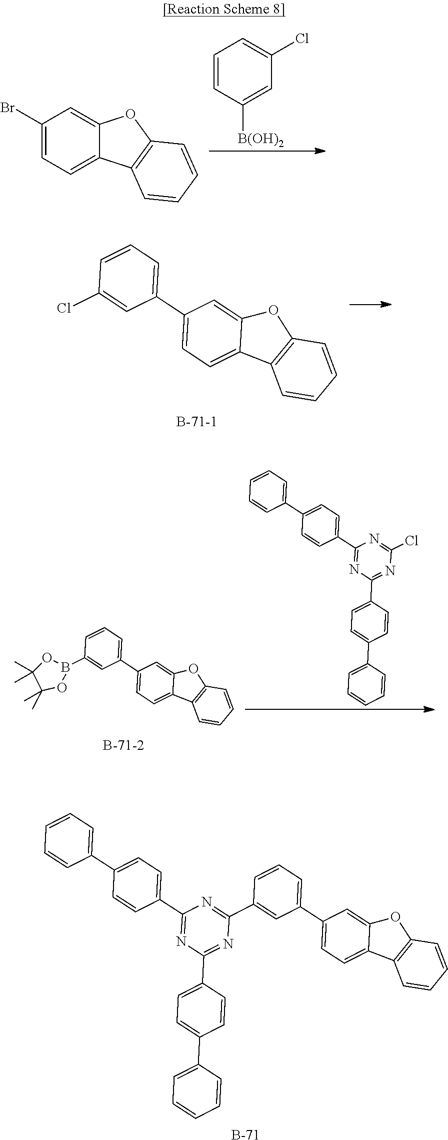

### a) Synthesis of Intermediate B-71-1

14.06 g (56.90 mmol) of 3-bromo-dibenzofuran, 200 mL of tetrahydrofuran, and 100 mL of distilled water were added in a 500 mL round-bottomed flask, 1 equivalent of 3′-chloro-phenylboronic acid, 0.03 equivalents of tetrakistriphenylphosphine palladium, and 2 equivalents of potassium carbonate were added thereto, and the mixture was heated and refluxed under a nitrogen atmosphere. After 18 hours, the reaction solution was cooled down, and a solid precipitated therein was filtered and washed with 500 mL of water. The solid was recrystallized with 500 mL of monochlorobenzene to obtain 12.05 g of Intermediate B-71-1. (a yield: 76%)

### b) Synthesis of Intermediate B-71-2

24.53 g (88.02 mmol) of the synthesized intermediate B-71-1 was added to DMF (250 mL) in a 500 mL round-bottomed flask, 0.05 equivalents of dichlorodiphenylphosphinoferrocene palladium, 1.2 equivalents of bispinacolato diboron, and 2 equivalents of potassium acetate were added thereto, and the mixture was heated and refluxed under a nitrogen atmosphere for 18 hours. The reaction solution was cooled down and then, added to 1 L of water in a dropwise fashion to obtain a solid. The obtained solid was dissolved in boiling toluene to treat activated carbon and then, filtered in silica gel and concentrated. The concentrated solid was stirred with a small amount of hexane and filtered to obtain 22.81 g of Intermediate B-71-2. (a yield: 70%)

### c) Synthesis of Compound B-71

Compound B-71 was synthesized according to the same method as a) of Synthesis Example 1 by using 1.0 equivalent of Intermediate B-71-2 and 1.0 equivalent of 2,4-bis([1,1′-biphenyl]-4-yl)-6-chloro-1,3,5-triazine.

LC/MS calculated for: C₄₅H₂₉N₃O Exact Mass: 627.2311 found for 628.25 [M+H]

### Synthesis Example 9: Synthesis of Compound B-129

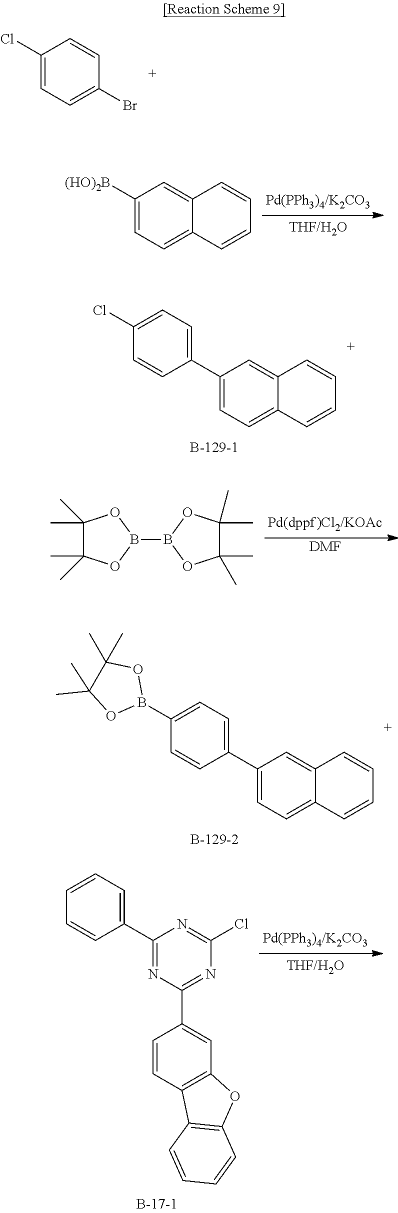

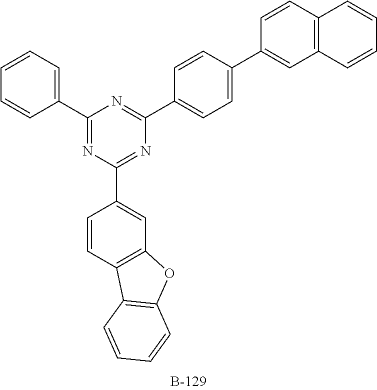 [SMILES: c1ccc(-c2nc(-c3ccc(-c4ccc5ccccc5c4)cc3)nc(-c3ccc4c(c3)oc3ccccc34)n2)cc1]

### a) Synthesis of Intermediate B-129-1

Intermediate B-129-1 was synthesized according to the same method as a) of Synthesis Example 8 by using 1-Bromo-4-chloro-benzene and 2-naphthalene boronic acid in each amount of 1.0 equivalent.

### b) Synthesis of Intermediate B-129-2

Intermediate B-129-2 was synthesized according to the same method as b) of Synthesis Example 8 by using intermediate B-129-1 and bis(pinacolato) diboron in a 1:1.2 equivalent ratio.

### c) Synthesis of Compound B-129

Compound B-129 was synthesized according to the same method as b) of Synthesis Example 1 by using intermediate B-129-2 and intermediate B-17-1 in each amount of 1.0 equivalent.

LC/MS calculated for: C₃₇H₂₃N₃O Exact Mass: 525.18 found for 525.22 [M+H]

(Preparation of Second Host)

### Synthesis Example 10: Synthesis of Compound HC-28

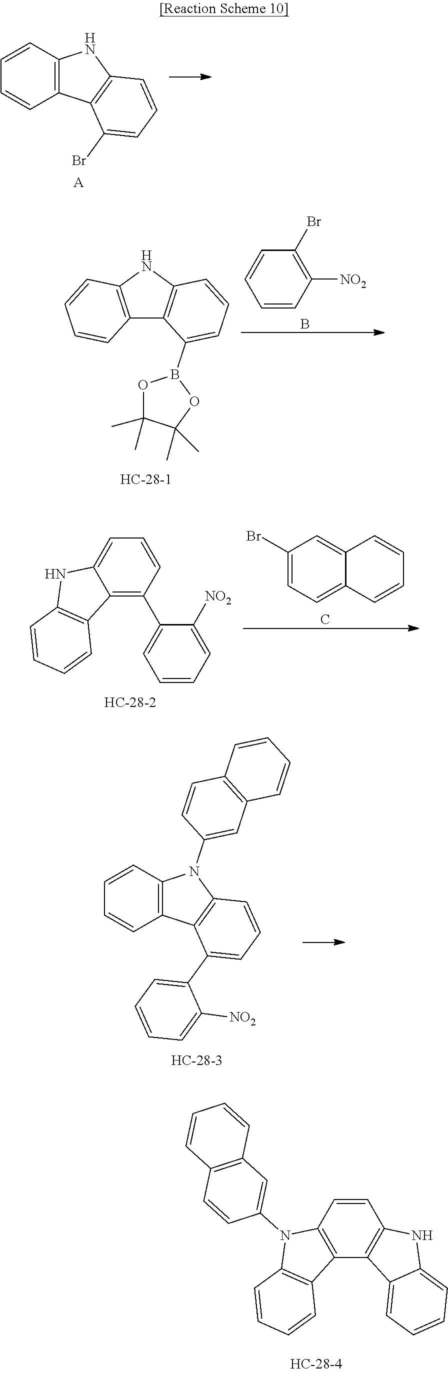

### a) Synthesis of Intermediate HC-28-1

Intermediate A (30 g, 121.9 mmol), 1 equivalent of 4,4,4′,4′,5,5,5′,5′-octamethyl-2,2′-bi(1,3,2-dioxaborolane), 2 equivalents of potassium acetate, and 0.03 equivalents of 1,1′-bis(diphenylphosphino) ferrocene-palladium (II) dichloride, and 0.2 equivalents of tricyclohexylphosphine were added to 300 mL of N,N-dimethylformamide in a 500 mL flask, and the mixture was stirred at 130° C. for 12 hours. When a reaction was complete, the reaction solution was extracted with water and EA to obtain an organic layer, magnesium sulfate was used to remove moisture therefrom, and the residue was concentrated and purified through column chromatography to obtain Intermediate HC-28-1 as a white solid (29.66 g, 83% of a yield).

### b) Synthesis of Intermediate HC-28-2

29.66 g (0.4 mol) of Intermediate HC-28-1, 2 equivalents of Intermediate B (1-bromo-2-nitro benzene), 2 equivalents of potassium carbonate, and 0.02 equivalent of tetrakis(triphenylphosphine) palladium (0) were added to 200 mL of 1,4-dioxane and 100 mL of water in a 500 mL flask, and the mixture was heated at 90° C. under a nitrogen flow for 16 hours. After removing the reaction solvent, a solid obtained therefrom was dissolved in dichloromethane, filtered with silica gel/Celite, and after removing an appropriate amount of the organic solvent, recrystallized with methanol to obtain Intermediate HC-28-2 as a solid (16.92 g, yield 58%).

### c) Synthesis of Intermediate HC-28-3

8.7 g (30.2 mmol) of Intermediate HC-28-2, 7.5 g (36.2 mmol) of Intermediate C (2-bromonaphthalene), 4.3 g (45.3 mmol) of sodium t-butoxide (NaOtBu), 1.0 g (1.8 mmol) of Pd(dba)₂, and 2.2 g of tri t-butylphosphine (P(tBu)₃) (50% in toluene) were put in 150 mL of xylene in a 500 mL flask and then, heated and refluxed under a nitrogen flow for 12 hours. After removing the xylene, 200 mL of methanol was added to a mixture obtained therefrom, a solid crystallized therein was filtered, dissolved in dichloromethane, filtered with silica gel/Celite, and after removing an appropriate amount of the organic solvent, recrystallized with acetone to obtain Intermediate HC-28-3 (9.83 g, yield 77%).

### d) Synthesis of Intermediate HC-28-4

211.37 g (0.51 mol) of Intermediate HC-28-3 and 528 ml (3.08 mol) of triethyl phosphate were put in a 1000 ml flask and was substituted with nitrogen, and the mixture was stirred for 12 hours at 160° C. When a reaction was complete, 3 L of MeOH was added thereto, the obtained mixture was filtered, and a filtrate therefrom was volatilized. The resultant was purified (hexane) through column chromatography to obtain Intermediate HC-28-4 (152.14 g, 78% of a yield).

### e) Synthesis of Compound HC-28

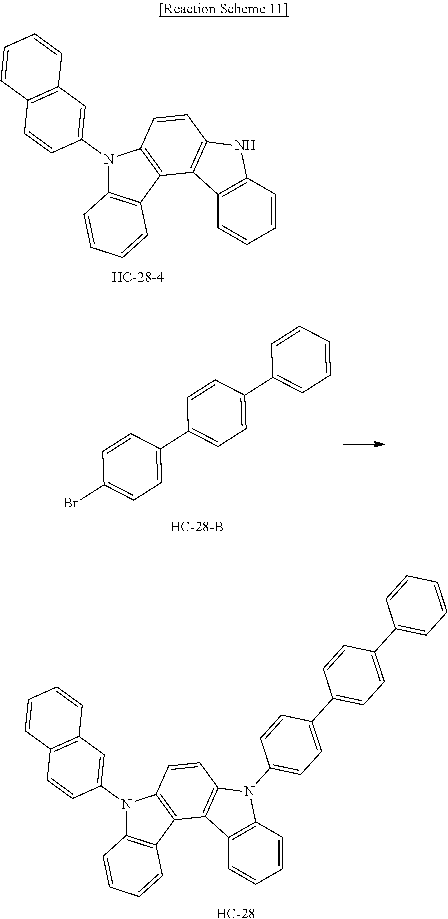

Compound HC-28 was synthesized according to the same method as c) of Synthesis Example 10 by using Intermediate HC-28-4 and Intermediate HC-28-B.

### Synthesis Example 11: Synthesis of Compound HC-30

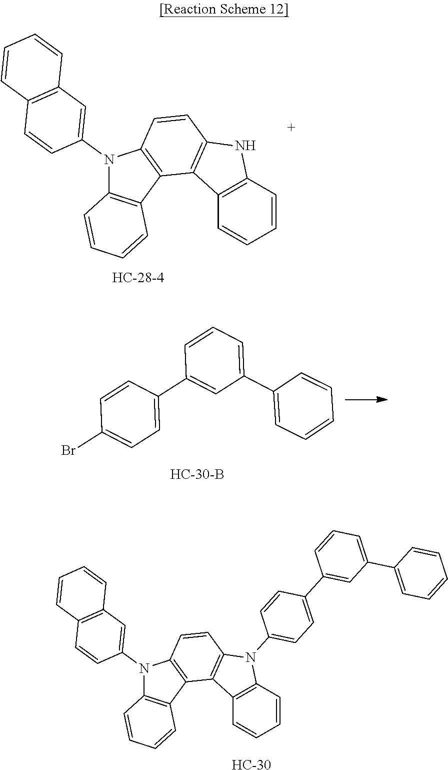

Compound HC-30 was synthesized according to the same method as e) of Synthesis Example 10 by using Intermediate HC-30-B instead of Intermediate HC-28-B.

### Synthesis Example 12: Synthesis of Compound HC-29

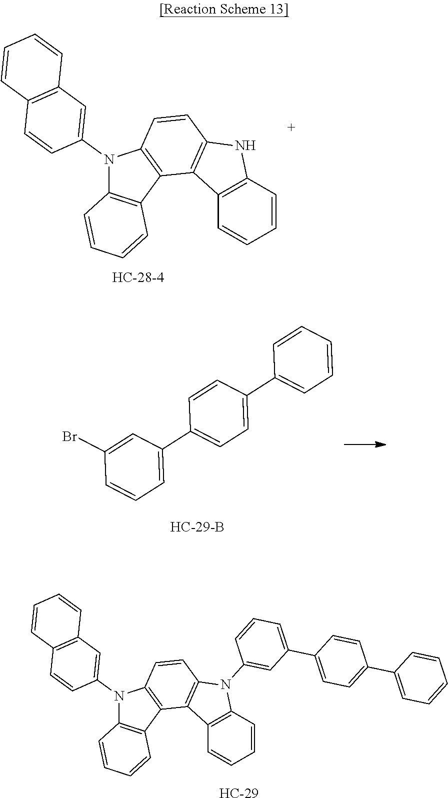

Compound HC-29 was synthesized according to the same method as e) of Synthesis Example 10 by using Intermediate HC-29-B instead of Intermediate HC-28-B.

### Synthesis Example 13: Synthesis of Compound HC-18

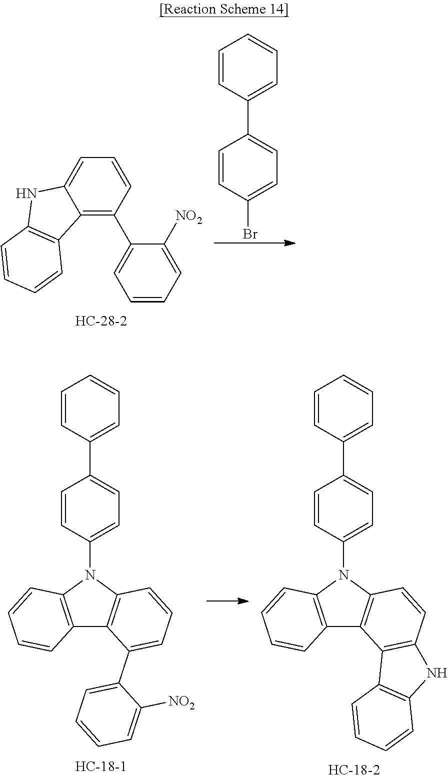 [SMILES: O=[N+]([O-])c1ccccc1-c1cccc2[nH]c3ccccc3c12 ; Brc1ccc(-c2ccccc2)cc1 ; O=[N+]([O-])c1ccccc1-c1cccc2c1c1ccccc1n2-c1ccc(-c2ccccc2)cc1 ; c1ccc(-c2ccc(-n3c4ccccc4c4c5c(ccc43)[nH]c3ccccc35)cc2)cc1]

### a) Synthesis of Intermediate HC-18-1

Intermediate HC-18-1 was synthesized according to the same method as c) of Synthesis Example 10 by using 4-bromobiphenyl as an intermediate instead of 2-bromonaphthalene.

### b) Synthesis of Intermediate HC-18-2

Intermediate HC-18-2 was synthesized according to the same method as d) of Synthesis Example 10.

### c) Synthesis of Intermediate HC-18-3

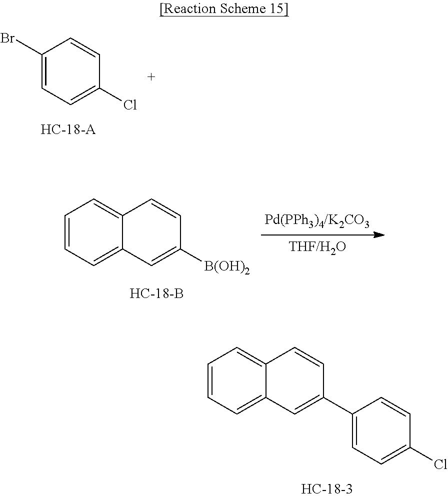

Intermediate HC-18-3 was synthesized according to the same method as b) of Synthesis Example 1 by using Intermediates HC-18-A and HC-18-B.

### d) Synthesis of Compound HC-18

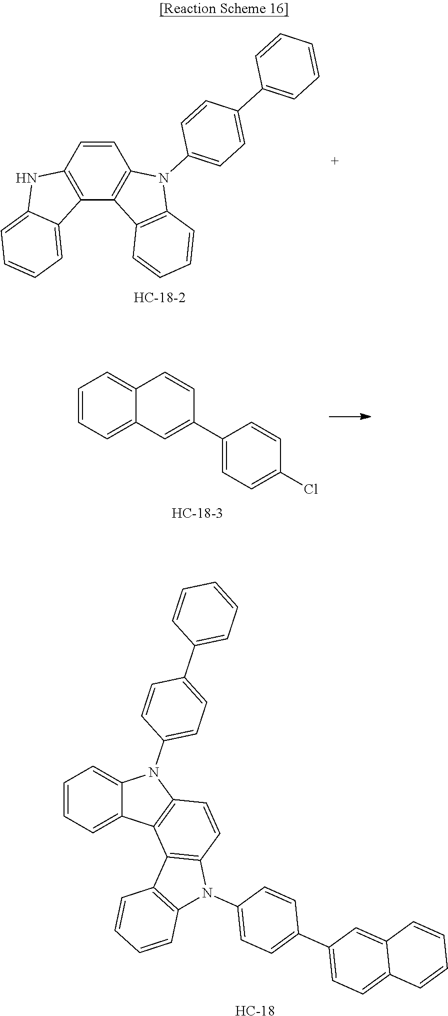

Compound HC-18 was synthesized according to the same method as e) of Synthesis Example 10 by using Intermediates HC-18-2 and HC-18-3.

### Reference Synthesis Example 1: Synthesis of Compound Ref.1

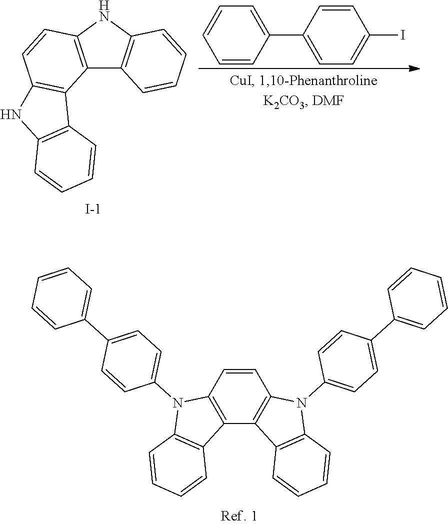

8 g (31.2 mmol) of Intermediate I-1, 20.5 g (73.32 mmol) of 4-iodobiphenyl, 1.19 g (6.24 mmol) of Cul, 1.12 g (6.24 mmol) of 1,10-phenanthoroline, and 12.9 g (93.6 mmol) of K₂CO₃ were put in a round-bottomed flask, 50 ml of DMF was added thereto, and the mixture was refluxed and stirred under a nitrogen atmosphere for 24 hours. When a reaction was complete, distilled water was added thereto for a precipitation, and a solid obtained therefrom was filtered. The solid was dissolved in 250 ml of xylene, filtered with silica gel, and precipitated into a white solid to obtain 16.2 g of a reference compound, Ref.1 (a yield of 93%).

(Manufacture of Organic Light Emitting Diode)

### Example 1

A glass substrate coated with ITO (indium tin oxide) as a 1500 Å-thick thin film was washed with distilled water. After washing with the distilled water, the glass substrate was ultrasonic wave-washed with a solvent such as isopropyl alcohol, acetone, methanol, and the like and dried and then, moved to a plasma cleaner, cleaned by using oxygen plasma for 10 minutes, and moved to a vacuum depositor. This obtained ITO transparent electrode was used as an anode, Compound A was vacuum-deposited on the ITO substrate to form a 700 Å-thick hole injection layer, Compound B was deposited to be 50 Å thick on the injection layer, and Compound C was deposited to be 700 Å thick to form a hole transport layer. A hole transport auxiliary layer was formed on the hole transport layer by depositing Compound C-1 in a thickness of 400 Å. A 400 Å-thick light emitting layer was formed on the hole transport auxiliary layer by vacuum-depositing Compound B-24 and Compound HC-28 simultaneously as hosts and 2 wt % of [Ir(piq)₂acac] as a dopant. Herein, Compound B-24 and Compound HC-28 were used in a 3:7 weight ratio. Subsequently, Compound D and Liq were vacuum-deposited simultaneously at a 1:1 ratio on the light emitting layer to form a 300 Å-thick electron transport layer and a cathode was formed by sequentially vacuum-depositing Liq to be 15 Å thick and Al to be 1200 Å thick on the electron transport layer, manufacturing an organic light emitting diode.

The organic light emitting diode had a five-layered organic thin layer, and specifically a structure of ITO/Compound A (700 Å)/Compound B (50 Å)/Compound C (700 Å)/Compound C-1 (400 Å)/EML[Compound B-24: Compound HC-28: [Ir(piq)₂acac] (2 wt %)] 400 Å/Compound D: Liq 300 Å/Liq 15 Å/Al 1200 Å.

Compound A: N4,N4′-d iphenyl-N4,N4′-bis(9-phenyl-9H-carbazol-3-yl)biphenyl-4,4′-diamine

Compound B: 1,4,5,8,9,11-hexaazatriphenylene-hexacarbonitrile (HAT-CN),

Compound C: N-(biphenyl-4-yl)-9,9-dimethyl-N-(4-(9-phenyl-9H-carbazol-3-yl)phenyl)-9H-fluoren-2-amine

Compound C-1: N,N-di([1,1′-biphenyl]-4-yl)-7,7-dimethyl-7H-fluoreno[4,3-b]benzofuran-10-amine

Compound D: 8-(4-(4,6-di(naphthalen-2-yl)-1,3,5-triazin-2-yl)phenyl)quinoline

### Examples 2 to 9 and Reference Examples 1 to 3

Organic light emitting diodes were respectively manufactured according to the same method as Example 1 by using the first and second hosts as shown in Table 1.

### Evaluation

Life-span characteristics of each organic light emitting diode according to Examples 1 to 9 and Reference Examples 1 to 3 were evaluated as follows and the results are shown in Table 1.

Measurement of Life-Span

T97 life-spans of the organic light emitting diodes according to Examples 1 to 9 and Reference Examples 1 to 3 were measured as a time when their luminance decreased down to 97% relative to the initial luminance (cd/m²) after emitting light with 9000 cd/m² as the initial luminance (cd/m²) and measuring their luminance decrease depending on a time with a Polanonix life-span measurement system. The results are shown as relative ratios with reference to 100% of life-span of Reference Example 1.

TABLE 1

| | First | Second | T97 |
|---|---|---|---|
| | host | host | life-span |
| Example 1 | B-24 | HC-28 | 307.5 |
| Example 2 | B-3 | HC-28 | 240 |
| Reference Example 1 | B-3 | Ref. 1 | 100 |
| Example 3 | B-23 | HC-28 | 172.5 |
| Reference Example 2 | B-23 | Ref. 1 | 62.5 |
| Example 4 | B-20 | HC-28 | 192.5 |
| Example 5 | B-124 | HC-28 | 270 |
| Example 6 | B-124 | HC-30 | 172.5 |
| Reference Example 3 | B-124 | Ref. 1 | 107.5 |
| Example 7 | B-71 | HC-18 | 145 |
| Example 8 | B-71 | HC-28 | 230 |
| Example 9 | B-129 | HC-28 | 417.5 |

Referring to Table 1, the organic light emitting diodes according to Examples 1 to 9 show remarkably improved life-span characteristics compared with the organic light emitting diodes according to Reference Examples 1 to 3.

While this invention has been described in connection with what is presently considered to be practical example embodiments, it is to be understood that the invention is not limited to the disclosed embodiments, but, on the contrary, is intended to cover various modifications and equivalent arrangements included within the spirit and scope of the appended claims. Therefore, the aforementioned embodiments should be understood to be exemplary but not limiting the present invention in any way.

1. An organic optoelectronic device, comprising:
an anode and a cathode facing each other, and
an organic layer disposed between the anode and the cathode,
wherein the organic layer includes an auxiliary layer including at least one of a hole injection layer, a hole transport layer, an electron injection layer, and an electron transport layer, and a light emitting layer, and
the light emitting layer includes a first host represented by Chemical Formula 1, a second host represented by a combination of Chemical Formula 2 and Chemical Formula 3, and a phosphorescent dopant having a photoluminescence wavelength of maximum emission of 550 nm to 750 nm: 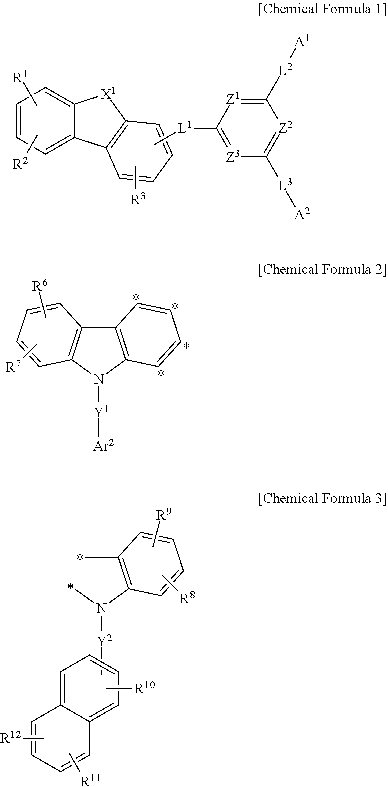
wherein, in Chemical Formula 1,
X¹ is O or S,
Z¹ to Z³ are independently N or CR^{a},
at least two of Z¹ to Z³ are N,
L¹ to L³ are independently a single bond, or a substituted or unsubstituted C6 to C20 arylene group,
A¹ and A² are independently a substituted or unsubstituted C6 to C30 aryl group, or a substituted or unsubstituted C2 to C30 heterocyclic group,
at least one of A¹ and A² is a substituted or unsubstituted C6 to C30 aryl group,
Rₐ and R¹ to R³ are independently hydrogen, deuterium, a cyano group, a substituted or unsubstituted C1 to C10 alkyl group, or a substituted or unsubstituted C6 to C20 aryl group;
wherein, in Chemical Formulae 2 and 3,
Ar² is a substituted or unsubstituted C6 to C20 aryl group,
two adjacent *'s of Chemical Formula 2 are carbon (C) linked with Chemical Formula 3,
* of Chemical Formula 2 that are not linked with Chemical Formula 3 are independently C-L^{a}-R^{b},
L^{a}, Y¹ and Y² are independently a single bond, or a substituted or unsubstituted C6 to C20 arylene group, and
R^{b} and R⁶ to R¹² are independently hydrogen, deuterium, a cyano group, a substituted or unsubstituted C1 to C10 alkyl group, or a substituted or unsubstituted C6 to C20 aryl group.

2. The organic optoelectronic device of claim 1, wherein the first host is represented by Chemical Formula 1-I: 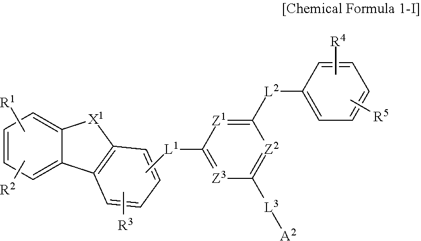
wherein, in Chemical Formula 1-I,
X¹ is O or S,
Z¹ to Z³ are independently N or CR^{a},
at least two of Z¹ to Z³ are N,
L¹ to L³ are independently a single bond, or a substituted or unsubstituted C6 to C20 arylene group,
A² is a substituted or unsubstituted C6 to C30 aryl group, or a substituted or unsubstituted C2 to C30 heterocyclic group, and
Rₐ and R¹ to R⁵ are independently hydrogen, deuterium, a cyano group, a substituted or unsubstituted C1 to C10 alkyl group, or a substituted or unsubstituted C6 to C20 aryl group.

3. The organic optoelectronic device of claim 1, wherein the first host is represented by one of Chemical Formula 1-I B-1 to Chemical Formula 1-I B-3: 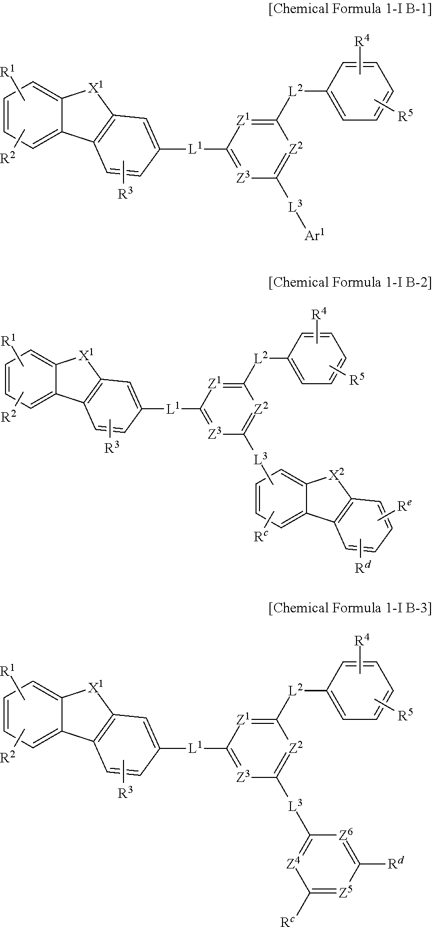
wherein, in Chemical Formulae 1-I B-1 to 1-I B-3,
Ar¹ is a substituted or unsubstituted C6 to C20 aryl group,
X¹ and X² are independently O or S,
Z¹ to Z⁶ are independently N or CR^{a},
at least two of Z¹ to Z³ are N,
at least two of Z⁴ to Z⁶ are N,
L¹ to L³ are independently a single bond, or a substituted or unsubstituted C6 to C20 arylene group, and
R^{a}, R^{c}, R^{d}, R^{e} and R¹ to R⁵ are independently hydrogen, deuterium, a cyano group, a substituted or unsubstituted C1 to C10 alkyl group, or a substituted or unsubstituted C6 to C20 aryl group.

4. The organic optoelectronic device of claim 1, wherein:
A¹ of Chemical Formula 1 is a substituted or unsubstituted C6 to C20 aryl group, and
A² of Chemical Formula 1 is a substituted or unsubstituted phenyl group, a substituted or unsubstituted biphenyl group, a substituted or unsubstituted naphthyl group, a substituted or unsubstituted terphenyl group, a substituted or unsubstituted quaterphenyl group, a substituted or unsubstituted dibenzofuranyl group, a substituted or unsubstituted dibenzothiophenyl group, a substituted or unsubstituted pyrimidinyl group, or a substituted or unsubstituted triazinyl group.

5. The organic optoelectronic device of claim 4, wherein:
A¹ of Chemical Formula 1 is selected from substituents of Group I, and
A² of Chemical Formula 1 is selected from substituents of Group II: 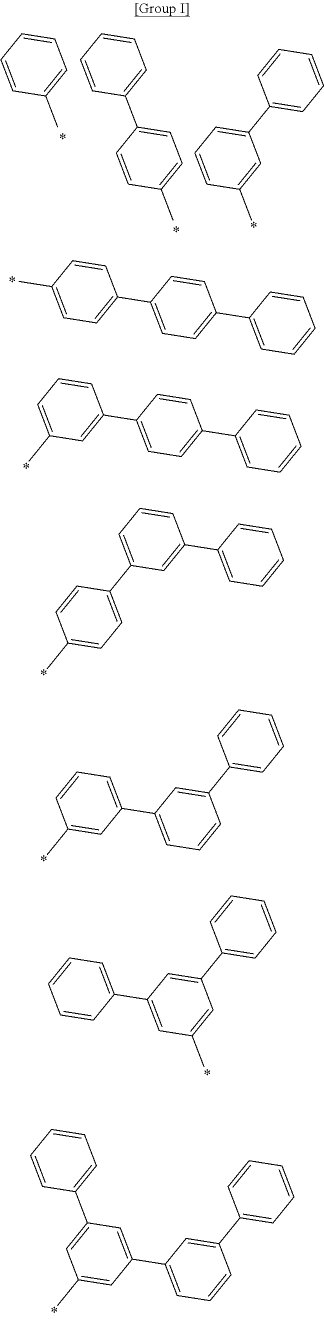 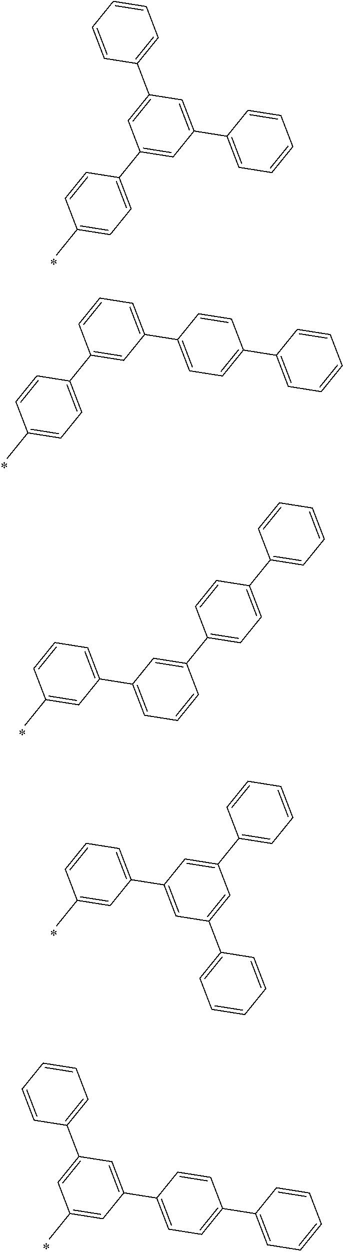 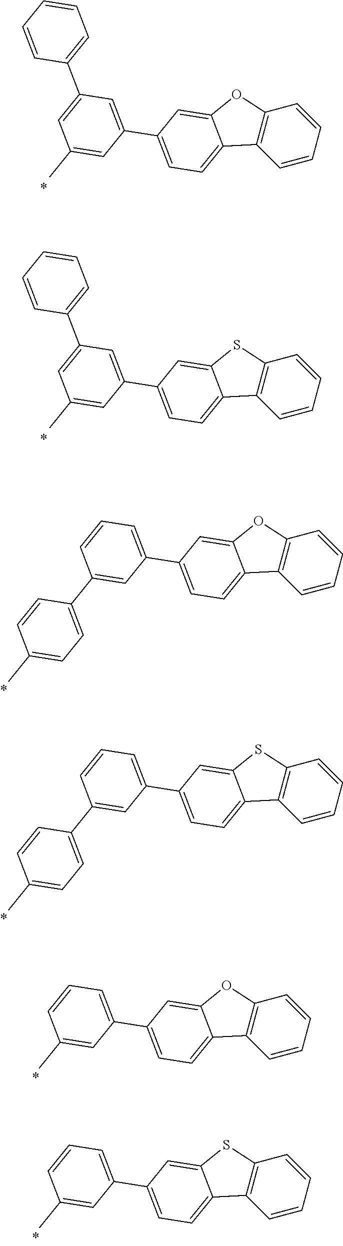 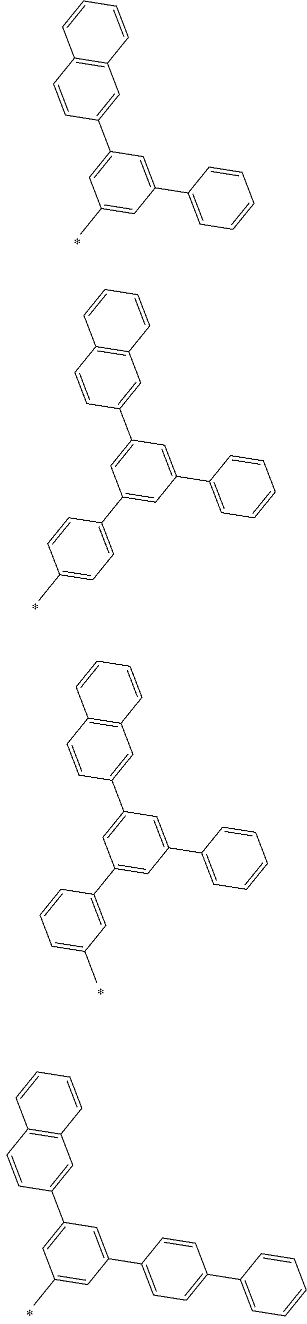 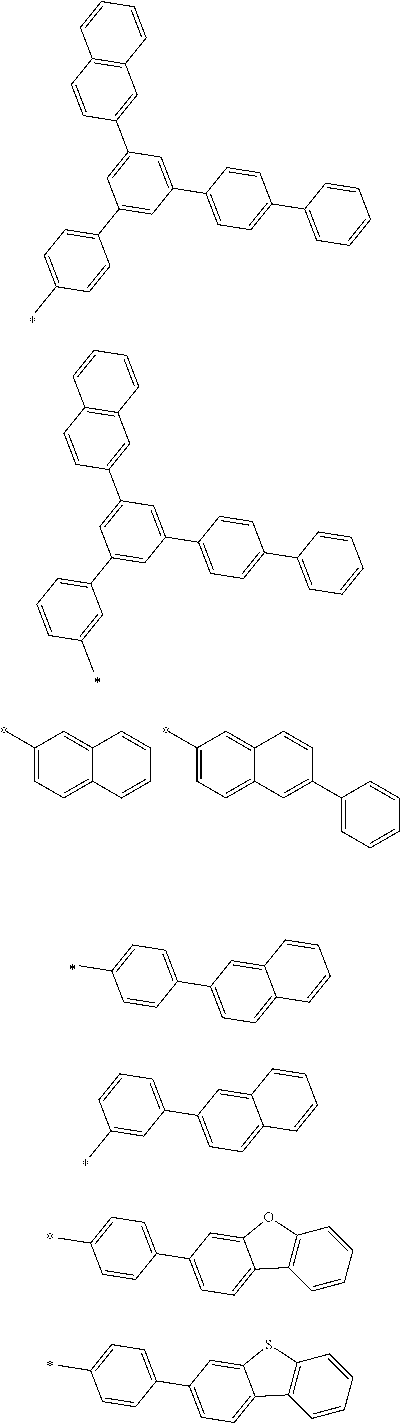 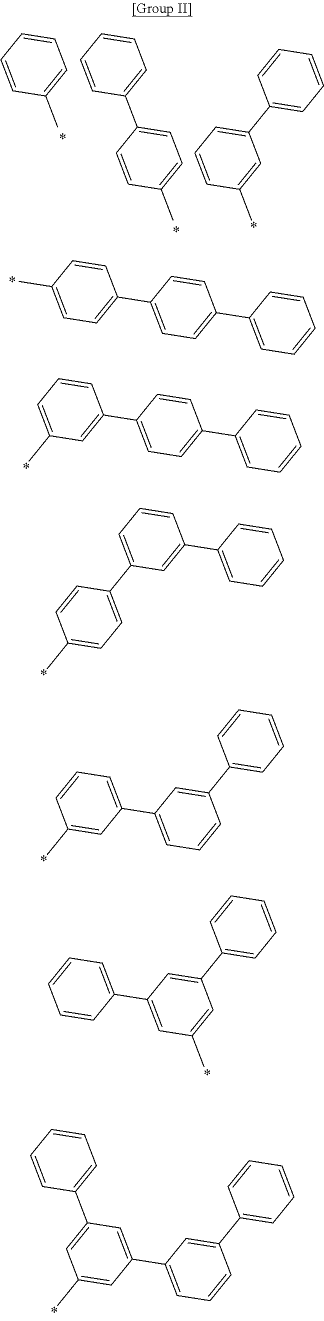 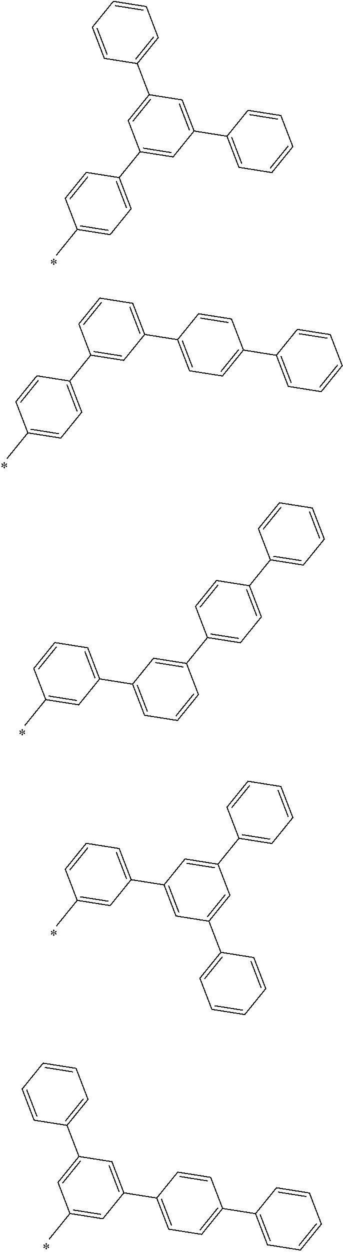 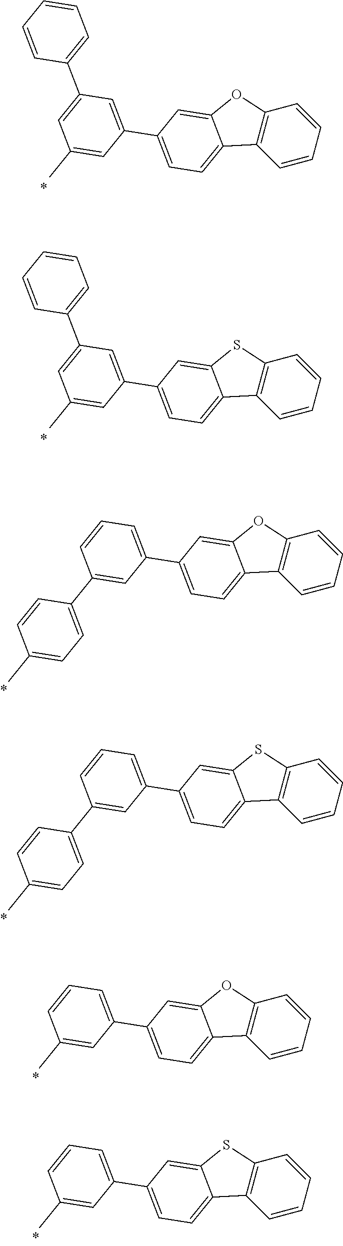 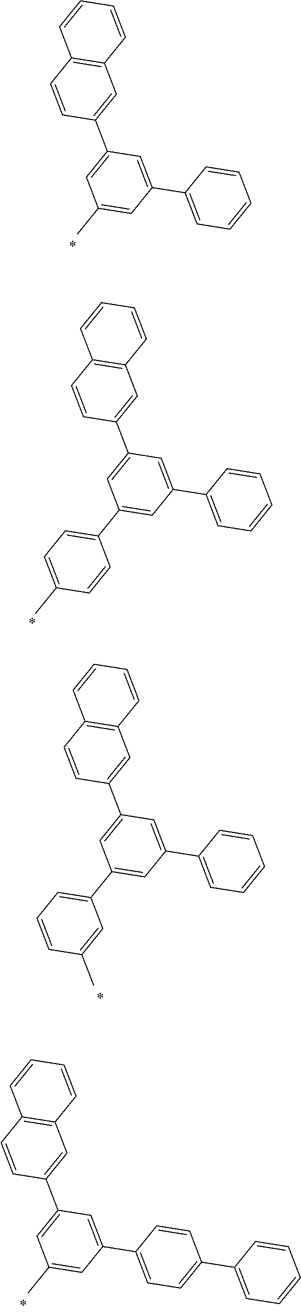 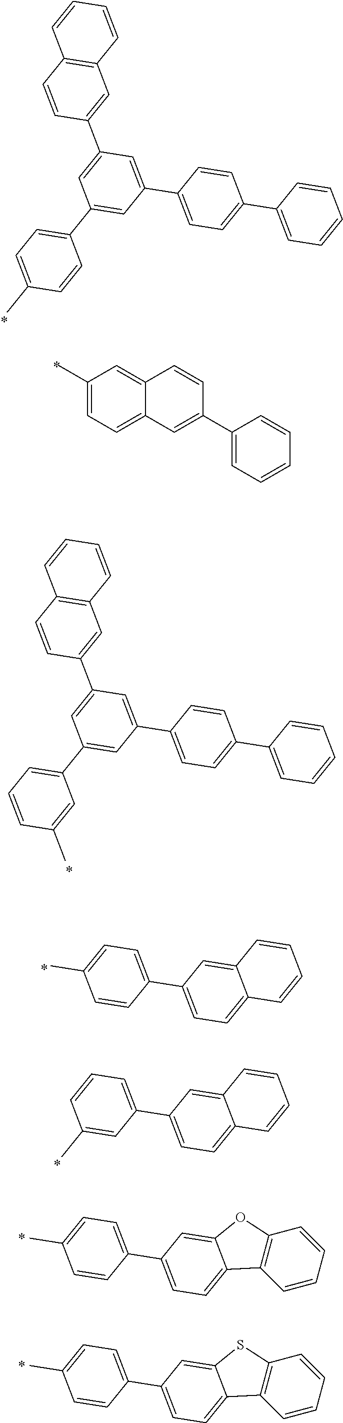 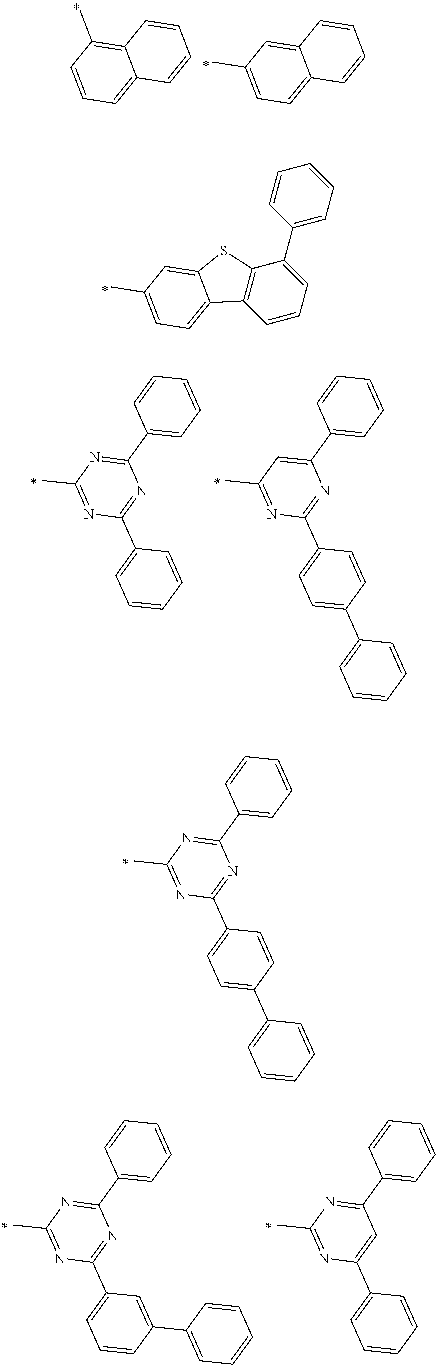 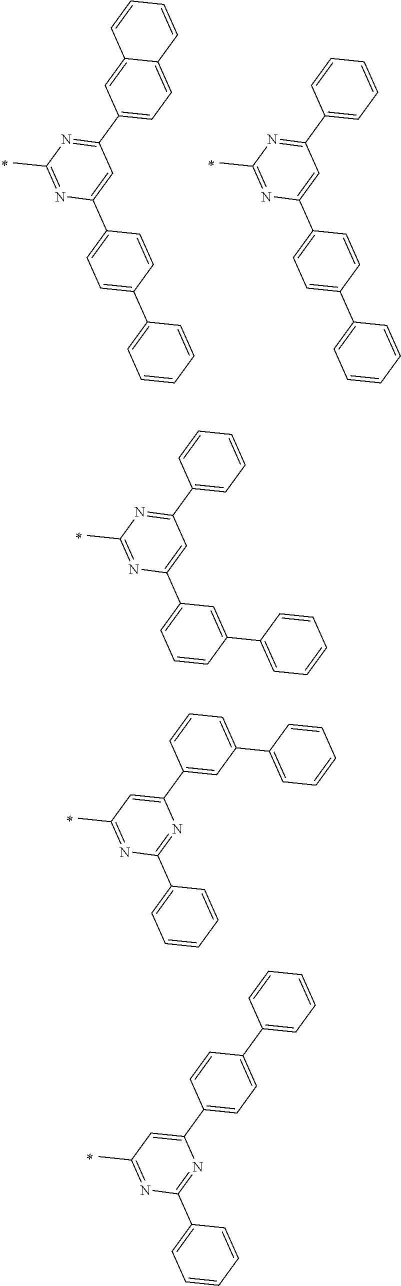 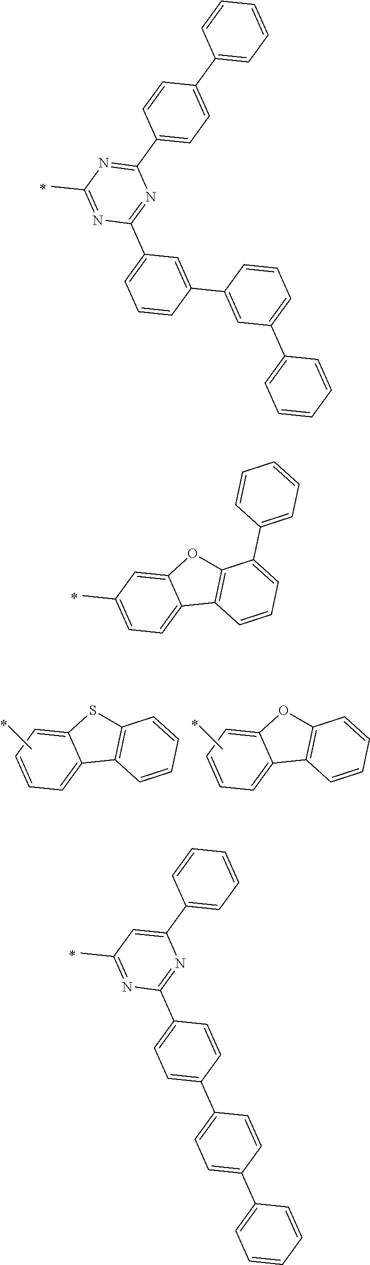 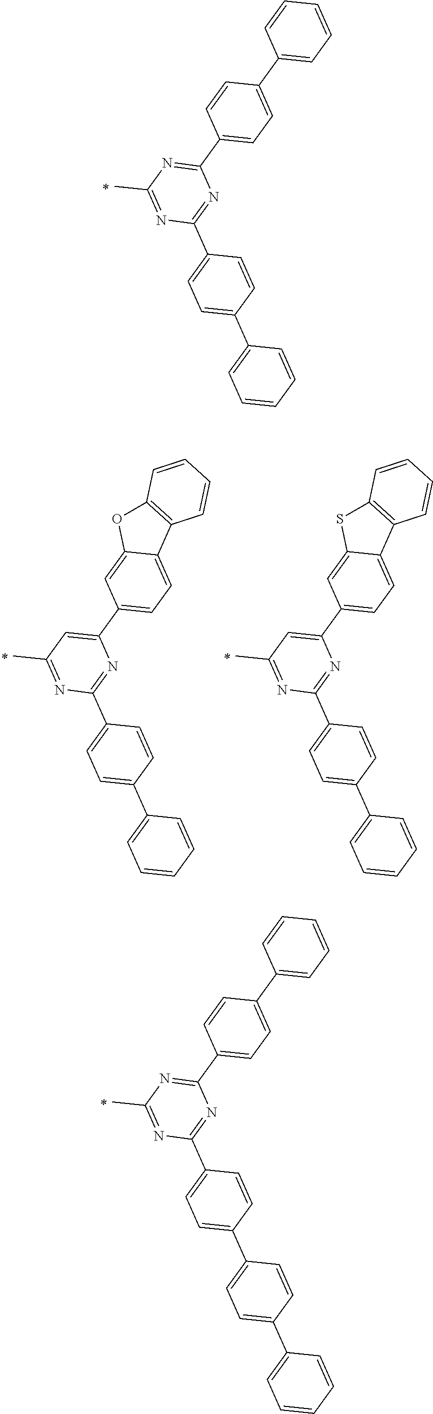 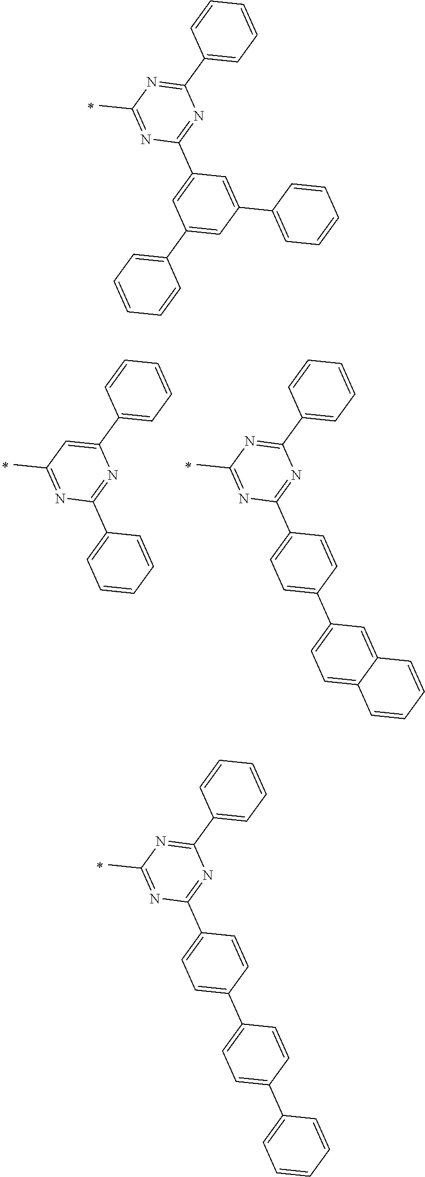 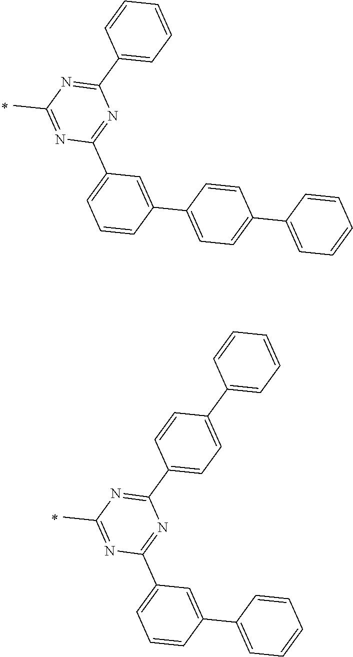
wherein,
in Group I, * is a linking point with L², and
in Group II, * is a linking point with L³.

6. The organic optoelectronic device of claim 1, wherein the second host is represented by Chemical Formula 2C: 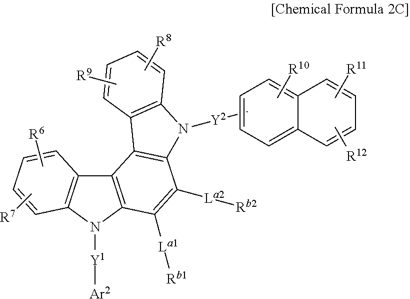
wherein, in Chemical Formula 2C,
Ar² is a substituted or unsubstituted C6 to C20 aryl group,
L^{a1} and L^{a2}, Y¹ and Y² are independently a single bond, or a substituted or unsubstituted C6 to C20 arylene group, and
R^{b1}, R^{b2} and R⁶ to R¹² are independently hydrogen, deuterium, a cyano group, a substituted or unsubstituted C1 to C10 alkyl group, or a substituted or unsubstituted C6 to C20 aryl group.

7. The organic optoelectronic device of claim 6, wherein Ar² is a substituted or unsubstituted phenyl group, a substituted or unsubstituted biphenyl group, a substituted or unsubstituted naphthyl group, or a substituted or unsubstituted terphenyl group.

8. The organic optoelectronic device of claim 1, wherein:
the first host is represented by Chemical Formula 1-I B-1 or Chemical Formula 1-I B-2, and
the second host is represented by Chemical Formula 2C-a: 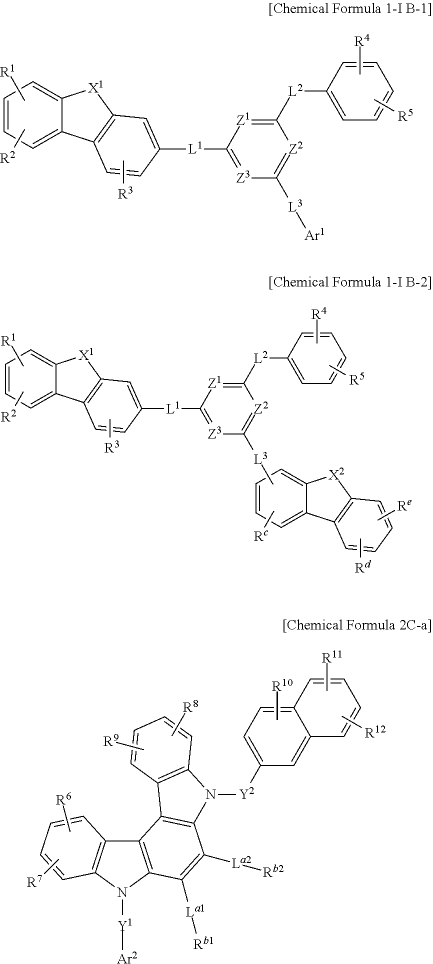
wherein, in Chemical Formula 1-I B-1, Chemical Formula 1-I B-2, and Chemical Formula 2C-a,
Ar¹ and Ar^{e} are independently, a substituted or unsubstituted phenyl group, a substituted or unsubstituted biphenyl group, a substituted or unsubstituted naphthyl group, a substituted or unsubstituted terphenyl group, or a substituted or unsubstituted quaterphenyl group,
X¹ and X² are independently O or S,
Z¹ to Z³ are independently N or CR^{a},
at least two of Z¹ to Z³ are N,
L¹ to L³, L^{a1}, L^{a2}, Y¹, and Y² are independently a single bond, or a substituted or unsubstituted C6 to C20 arylene group, and
R^{a}, R^{b1}, R^{b2}, R^{c}, R^{d}, R^{e}, R¹, R⁵, and independently hydrogen, deuterium, a cyano group, a substituted or unsubstituted C1 to C10 alkyl group, or a substituted or unsubstituted C6 to C20 aryl group.

9. The organic optoelectronic device of claim 1, wherein the phosphorescent dopant having the photoluminescence wavelength of maximum emission of 550 nm to 750 nm is an iridium (Ir) complex or a platinum (Pt) complex.

10. The organic optoelectronic device of claim 1, wherein the phosphorescent dopant includes a platinum (Pt) complex represented by Chemical Formula 4-1: 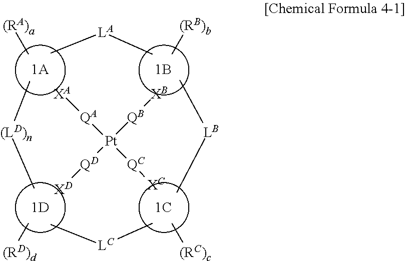
wherein, in Chemical Formula 4-1,
X^{A}, X^{B}, X^{C}, and X^{D} are elements that form unsaturated rings with each of 1A, 1B, 1C, and 1D, and independently C or N,
1A, 1B, 1C, and 1D are independently a substituted or unsubstituted C6 to C30 aryl group, or a substituted or unsubstituted C2 to C30 heterocyclic group,
L^{A}, L^{B}, L^{C}, L^{D}, Q^{A}, Q^{B}, Q^{C}, and Q^{D} are independently a single bond, O, S, a substituted or unsubstituted C1 to C30 alkylene group, a substituted or unsubstituted C2 to C30 alkenylene group, a substituted or unsubstituted C6 to C30 arylene group, or a substituted or unsubstituted C2 to C30 heteroarylene group,
R^{A}, R^{B}, R^{C}, and R^{D} are independently hydrogen, deuterium, a cyano group, a halogen, silane group, phosphine group, amine group, a substituted or unsubstituted C1 to C30 alkyl group, a substituted or unsubstituted C6 to C30 aryl group, or a substituted or unsubstituted C2 to C30 heteroaryl group,
R^{A}, R^{B}, R^{C}, and R^{D} are independently present or adjacent groups are linked with each other to form a ring,
n is one of integers of 0 to 5, and
a, b, c, and d are independently one of integers of 0 to 3.

11. The organic optoelectronic device of claim 1, wherein the phosphorescent dopant includes an iridium (Ir) complex represented by Chemical Formula 4-2: 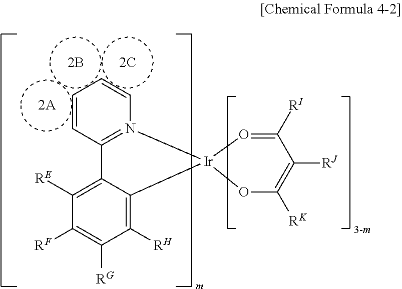
wherein, in Chemical Formula 4-2,
2A, 2B, and 2C are independently a substituted or unsubstituted benzene ring,
at least one of 2A, 2B, and 2C forms a fused ring with an adjacent complex compound,
R^{E}, R^{F}, R^{G}, R^{H}, R^{I}, R^{J}, and R^{K} are independently hydrogen, deuterium, a cyano group, a halogen, silane group, phosphine group, amine group, a substituted or unsubstituted C1 to C10 alkyl group, a substituted or unsubstituted C6 to C30 aryl group, or a substituted or unsubstituted C2 to C30 heteroaryl group,
R^{E}, R^{F}, R^{G}, R^{H}, R^{I}, R^{J}, and R^{K} are independently present or adjacent groups are linked with each other to form a ring, and
m is one of integers of 1 to 3.

12. A composition for a red phosphorescent host, comprising:
a first host represented by Chemical Formula 1, and
a second host represented by a combination of Chemical Formula 2 and Chemical Formula 3: 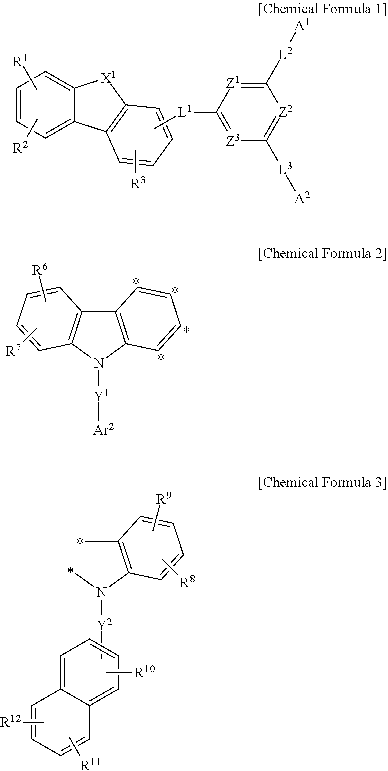
wherein, in Chemical Formula 1,
X¹ is O or S,
Z¹ to Z³ are independently N or CR^{a},
at least two of Z¹ to Z³ are N,
L¹ to L³ are independently a single bond, or a substituted or unsubstituted C6 to C20 arylene group,
A¹ and A² are independently a substituted or unsubstituted C6 to C30 aryl group, or a substituted or unsubstituted C2 to C30 heterocyclic group,
at least one of A¹ and A² is a substituted or unsubstituted C6 to C30 aryl group,
R^{a} and R¹ to R³ are independently hydrogen, deuterium, a cyano group, a substituted or unsubstituted C1 to C10 alkyl group, or a substituted or unsubstituted C6 to C20 aryl group,
wherein, in Chemical Formulae 2 and 3,
Ar² is a substituted or unsubstituted C6 to C20 aryl group,
two adjacent *'s of Chemical Formula 2 are carbon (C) linked with Chemical Formula 3,
* of Chemical Formula 2 that are not linked with Chemical Formula 3 are independently C-L^{a}-R^{b},
L^{a}, Y¹ and Y² are independently a single bond, or a substituted or unsubstituted C6 to C20 arylene group, and
R^{b} and R⁶ to R¹² are independently hydrogen, deuterium, a cyano group, a substituted or unsubstituted C1 to C10 alkyl group, or a substituted or unsubstituted C6 to C20 aryl group.

13. The composition for a red phosphorescent host of claim 12, wherein:
the first host is represented by Chemical Formula 1-I, and
the second host is represented by Chemical Formula 2C: 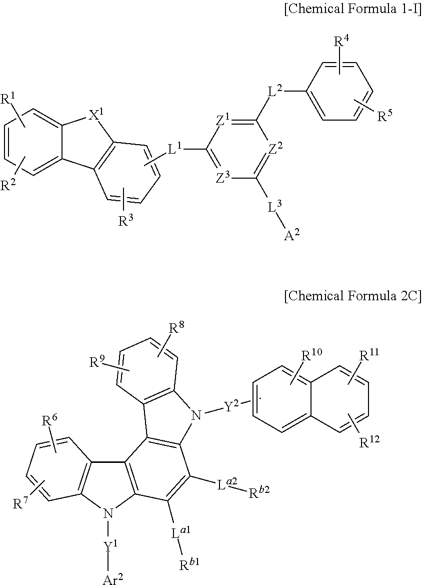
wherein, in Chemical Formula 1-I and Chemical Formula 2C,
X¹ is O or S,
Z¹ to Z³ are independently N or CR^{a},
at least two of Z¹ to Z³ are N,
L¹ to L³, L^{a1}, L^{a2}, Y¹, and Y² are independently a single bond, or a substituted or unsubstituted C6 to C20 arylene group,
A² is a substituted or unsubstituted C6 to C30 aryl group, or a substituted or unsubstituted C2 to C30 heterocyclic group,
Ar² is a substituted or unsubstituted C6 to C30 aryl group, and
Rₐ, R^{b1}, R^{b2}, and R¹ to R¹² are independently hydrogen, deuterium, a cyano group, a substituted or unsubstituted C1 to C10 alkyl group, or a substituted or unsubstituted C6 to C20 aryl group.

14. The composition for a red phosphorescent host of claim 13, wherein the first host is represented by Chemical Formula 1-I B-1 or Chemical Formula 1-I B-2: 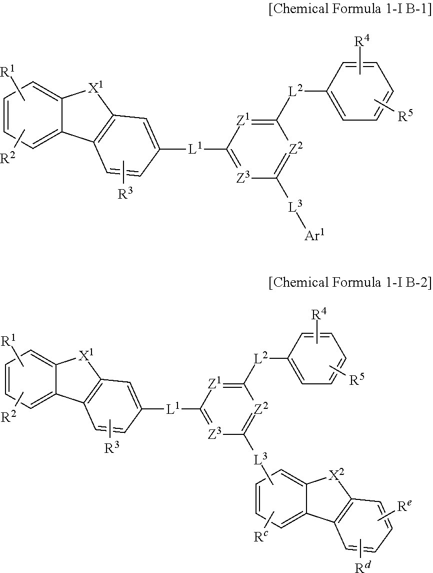
wherein, in Chemical Formula 1-I B-1, Chemical Formula 1-I B-2,
Ar¹ is a substituted or unsubstituted phenyl group, a substituted or unsubstituted biphenyl group, a substituted or unsubstituted naphthyl group, a substituted or unsubstituted terphenyl group, or a substituted or unsubstituted quaterphenyl group,
X¹ and X² are independently O or S,
Z¹ to Z³ are independently N or CR^{a},
at least two of Z¹ to Z³ are N,
L¹ to L³ are independently a single bond, or a substituted or unsubstituted C6 to C20 arylene group, and
R^{c}, R^{d}, R^{e} and R¹ to R⁵ are independently hydrogen, deuterium, a cyano group, a substituted or unsubstituted C1 to C10 alkyl group, or a substituted or unsubstituted C6 to C20 aryl group.

15. A display device comprising the organic optoelectronic device of claim 1.
